(19) United States
(12) Patent Application Publication
(10) Pub. No.: US 2024/0309347 A1
(43) Pub. Date: Sep. 19, 2024

(54) Fusion Proteins for CRISPR-based Transcriptional Repression

(71) Applicant: Dharmacon, Inc., Lafayette, CO (US)

(72) Inventors: CLARENCE MILLS, Denver, CO (US); ZAKLINA STREZOSKA, Westminster, CO (US); JOHN SCHIEL, Westminster, CO (US)

(21) Appl. No.: 18/275,442

(22) PCT Filed: Feb. 4, 2022
(86) PCT No.: PCT/US22/15162

§ 371 Date: Aug. 2, 2023

Related U.S. Application Data
(60) Provisional application No. 63/146,419, filed on Feb. 5, 2021.

(51) Int. Cl. C12N 9/22; A61K 38/00; C07K 14/47; C12N 15/113

(52) U.S. Cl. CPC C12N 9/22; C07K 14/4703; C12N 15/113; A61K 38/00; C07K 2319/70; C07K 2319/80; C12N 2310/20

(57) ABSTRACT

The present disclosure provides compositions for modulating the expression of a nucleic acid and methods for using these compositions. The compositions comprise fusion proteins that contain the repressor domain for one or both of SALL1 and SUDS3. In some embodiments, the compositions are Cas fusion proteins that may be used in combination with a gRNA or other RNA. Additionally or alternatively, the compositions are RNA-repressor domain complexes.

### CROSS-REFERENCE TO RELATED APPLICATION

This application is a national stage application of international application serial number PCT/US2022/015162, filed Feb. 4, 2022, which claims the benefit of the filing date of U.S. Provisional Application Ser. No. 63/146,419, filed Feb. 5, 2021, the entire disclosures of which are incorporated by reference as if set forth fully herein.

### FIELD OF THE INVENTION

The present invention relates to the field of CRISPR based transcriptional repression.

### BACKGROUND OF THE INVENTION

The biotechnology community is now familiar with the CRISPR-Cas9 system, which allows for specific targeting and editing of genes. This system was originally discovered within archaea and bacteria, but the great promise is for human applications.

The basic CRISPR/Cas9 system comprises a Cas9 protein and a guide RNA (“gRNA”). A spacer sequence (also referred to as a targeting sequence) within the gRNA leads the Cas9 protein to a genomic target site based on the complementarity between the spacer sequence and a sequence at the target site. After the Cas9 protein is brought to the target site it can cleave the target DNA and lead to DNA editing. Alternatively, a deactivated Cas9 (“dCas9”) can be used for sequence-specific targeting and bringing other effectors with different functionalities.

Because the CRISPR/Cas9 system is effective at locating and editing target sites, researchers have explored ways to piggyback on this system in order to introduce functions other than those that might be caused by the naturally occurring Cas9 protein's active sites. Further, researchers have begun to explore the use of other Cas proteins that rely on the specificity of gRNAs in order to bring those proteins to target sites.

Although researchers originally discovered CRISPR-Cas9 systems in lower organisms, the systems have successfully been used for gene editing applications in mammalian cells, M. Jinek, et al., “A programmable dual-RNA-guided DNA endonuclease in adaptive bacterial immunity,” Science. 337: p. 816-821 (2012). Further, researchers have been able to abolish the nuclease activity of the Cas protein by point mutations that are introduced into the catalytic residues (D10A and H840A in the case of the commonly used Streptococcus pyogenes Cas9 protein) yielding a deactivated Cas9 that maintains the ability to bind to target DNA when guided by sequence-specific guide RNAs. When the dCas9 is fused to transcriptional regulators and guided to gene promoter regions, it induces RNA-directed transcriptional regulation. CRISPR-based technologies for transcriptional regulation include CRISPR interference (CRISPRi) for transcriptional repression and CRISPR activation (CRISPRa) for transcriptional upregulation (Qi, L. S., et al., “Repurposing CRISPR as an RNA-guided platform for sequence-specific control of gene expression,” Cell, 152(5): p. 1173-83 (2013); A. W. Cheng, et al., “Multiplexed activation of endogenous genes by CRISPR-on, an RNA-guided transcriptional activator system,” Cell Res., 23(10): p. 1163-71 (2013)).

One known CRISPR-based approach for transcriptional repression utilizes the Krüppel associated box (KRAB) domain from zinc finger protein 10 (KOX1) as a transcriptional repressor, L. A. Gilbert, et al., “CRISPR-mediated modular RNA-guided regulation of transcription in eukaryotes,” Cell, 154(2): p. 442-51 (2013); L. A. Gilbert et al., “Genome-scale CRISPR-mediated control of gene repression and activation,” Cell, 159: p. 647-661 (2014). However, this approach has its limitations. Researchers have shown that it does not provide sufficient repression in all applications, and use of it can result in less robust repression of the target gene(s), L. Stojic et al., “Specificity of RNAi, LNA and CRISPRi as loss-of-function methods in transcriptional analysis,” Nucleic Acids Research, 46(12): p. 5950-5966 (2018); Yeo, et al., “An enhanced CRISPR repressor for targeted mammalian gene regulation,” Nat. Methods, 15(8): p. 611-616 (2018). Given the reported variability in performance of the CRISPR-KRAB fusion protein, which is the most commonly used fusion protein for transcriptional repression, there is a need for additional CRISPR-based approaches for transcriptional repression.

### SUMMARY OF THE INVENTION

The present invention provides novel fusion proteins, nucleic acid sequences that encode those proteins, and methods of gene repression by using those proteins and/or nucleic acids. Through the use of various embodiments of the present invention, one may efficiently and effectively regulate gene expression.

According to a first embodiment, the present invention provides a Cas fusion protein comprising a Cas protein and one or both of a SALL1 repressor domain and a SUDS3 repressor domain. In some embodiments the Cas protein is deactivated, which also may be referred to as dead or attenuated.

According to a second embodiment, the present invention provides a nucleic acid encoding a Cas fusion protein of the present invention.

According to a third embodiment, the present invention provides an RNA-repressor domain complex. The RNA-repressor domain complex comprises: (a) a gRNA molecule, wherein the gRNA molecule contains 30 to 180 nucleotides; (b) a ligand binding moiety, wherein the ligand binding moiety is either (i) directly bound to the gRNA molecule, or (ii) bound through a ligand binding moiety linker to the gRNA molecule; (c) a ligand, wherein the ligand is capable of reversibly associating with the ligand binding moiety; and (d) a fusion protein, wherein the fusion protein comprises a SALL1 repressor domain and a SUDS3 repressor domain, and wherein the fusion protein is either (i) directly bound to the ligand, or (ii) bound through a linker to the ligand.

According to a fourth embodiment, the present invention provides a method of modulating expression of a target nucleic comprising introducing a Cas fusion protein or an RNA-repressor domain complex of the present invention or a nucleic acid of the present invention into a cell such as a eukaryotic cell or an organism such as a mammal, e.g., a human. In some embodiments, introduction is in vivo, in vitro, or ex vivo.

According to a fifth embodiment, the present invention provides a kit comprising a Cas fusion protein of the present invention or a nucleic acid encoding a Cas fusion protein of the present invention and in some embodiments may further comprise either a gRNA or a nucleic acid that encodes for a gRNA.

According to a sixth embodiment, the present invention provides a kit comprising an RNA-repressor domain complex, or a nucleic acid encoding, two molecules, an RNA-ligand binding domain and ligand-repressor of the present invention.

According to a seventh embodiment, the present invention provides a protein that comprises, consists essentially of, or consists of a sequence at least 80% similar to SEQ ID NO: 10.

### BRIEF DESCRIPTION OF THE FIGURES

FIG. 1 is a representation of Cas fusion protein of the present invention associated with a single guide RNA (“sgRNA”) and a target DNA.

FIG. 2 is an example of an sgRNA that may be used in various embodiments of the present invention.

FIG. 3A is a graph that depicts gene knockdown in K562 cells nucleofected with either dCas9-KRAB or dCas9-SALL1-SUDS3 mRNA. FIG. 3B is a graph that depicts gene knockdown in Jurkat cells nucleofected with either dCas9-KRAB or dCas9-SALL1-SUDS3 mRNA. FIG. 3C is a graph that depicts gene knockdown in U2OS cells nucleofected with either dCas9-KRAB and dCas9-SALL1-SUDS3 mRNA.

FIG. 4 is a graph that compares repression of target genes when dCas9-SALL1-SUDS3 eGFP mRNA is introduced into HCT 116 cells to repression of target genes when dCas9-KRAB eGFP mRNA is introduced into HCT 116 cells. The genes are targeted with a pool of three synthetic sgRNAs delivered at 25 nM. Cells were sorted at 24 hours post-transfection into two categories: GFP negative (GFP Neg), and top 10% GFP expressing (Top 10%), and after 24 hours of recovery analyzed for transcriptional repression of the targeted genes.

FIGS. 5A-5C compare repression in systems that contain dCas9-KRAB versus systems that contain dCas9-SALL1-SUDS3 in different cell lines: U2OS (FIG. 5A); Jurkat (FIG. 5B); and hiPS stable hEF1α (FIG. 5C).

FIG. 6A shows gene repression by dCas9-KRAB and dCas9-SALL1-SUDS3 against BRCA1, PSMD7, SEL1L, and ST3GAL4 in K562 cells. FIG. 6B shows gene repression by dCas9-KRAB and dCas9-SALL1-SUDS3 against BRCA1, PSMD7, SEL1L, and ST3GAL4 in A375 cells.

FIGS. 7A-7D compare the repression by dCas9-KRAB to repression by dCas9-SALL1-SUDS3 over a course of six days in U2OS cells for different gene targets: BRCA1 (FIG. 7A); CD46 (FIG. 7B); HBP1 (FIG. 7C); and SEL1L (FIG. 7D).

FIG. 8A shows repression using individual sgRNAs against PPIB, SEL1L, and RAB11A and pools of sgRNAs against these targets when introduced with Cas fusion proteins of the present invention. FIG. 8B shows the repression of BRCA1, PSMD7, SEL1L, and ST3GAL4 by either individual sgRNAs or pools of sgRNAs against these targets when introduced with Cas fusion proteins of the present invention.

FIG. 9 shows expression of the following genes: PPIB, RAB11A, and SEL1, in hiPSC cells in the presence of gRNAs and dCas9-SALL1-SUDS3 when multiplexing, i.e., using sgRNAs against multiple genes.

FIG. 10 is a graph that shows functional phenotype of the repression of PSMD3, PSMD8, and PSMD11 genes in U2OS-Ubi (G76V)-EGFP reporter cell line in the presence of gRNAs and dCas9 fused to KRAB or SALL1-SUDS3 at the N terminal amino acid of the dCas9 or the C terminal amino acid of dCas9.

FIG. 11 is a graph of transcriptional repression in systems with a plasmid expressing gRNA and a plasmid expressing a fusion protein co-transfected in A375 cells.

FIG. 12 is a graph of transcriptional repression in systems with a plasmid expressing gRNA and a plasmid expressing a fusion protein co-transfected in U2OS cells.

FIG. 13 is a graph that shows the effect of combining SALL1 or SUDS3 each with an additional repressor domain.

FIG. 14A is a representation of repression by dMAD7-SALL1-SUDS3 as compared to dMAD7 in U2OS cells. FIG. 14B is a representation of repression by dCasPhi8-SALL1-SUDS3 as compared to dCasPhi8 in U2OS cells.

FIG. 15A is a diagram of the effect of using sgRNAs of different crRNA-targeting sizes with Cas9 that is not deactivated for simultaneous repression and gene editing. FIG. 15B is a graph that depicts the measurement of repression of MRE11a while LBR is simultaneously edited. FIG. 15C is a graph that depicts the measurement of repression of MRE11a while PPIB is simultaneously edited. FIG. 15D is a graph that depicts the measurement of repression of SEL1L while LBR is simultaneously edited. FIG. 15E is a graph that depicts the measurement of repression of SEL1L while PPIB is simultaneously edited.

FIG. 16 is a graph of repression effects of systems that contain single repressor dCas9 fusion proteins in the U2OS-Ubi (G76V)-EGFP reporter cell line.

FIG. 17 is a graph that compares the transcriptional repression in U2OS cells stably expressing dCas9-KRAB, dCas9-KRAB MeCP2, or dCas9-SUDS3 that were transfected with synthetic guide RNAs.

FIG. 18A is a representation of the phenotypic effects of gene knockdown in U2OS Ubi[G76V]-EGFP reporter cells expressing either dCas9-KRAB or dCas9-SALL1-SUDS3 and transfected with synthetic guides targeting proteasome genes. FIG. 18B depicts the corresponding transcriptional repression of the targeted proteasome genes.

FIG. 19A shows the transcriptional repression of PPIB and SEL1L in U2OS cells stably expressing either dCas9-SALL1-SUDS3 and a guide RNA from a single lentiviral vector or from two separate vectors. FIG. 19B shows the transcriptional repression of PPIB and SEL1L in HCT 116 cells stably expressing either dCas9-SALL1-SUDS3 and a guide RNA from a single lentiviral vector or from two separate vectors.

FIG. 20A shows the transcriptional repression of BRCA1, PSMD7, SEL1L, and ST3GAL4 by either synthetic or plasmid sgRNAs in U2OS cells stably expressing dCas9-SALL1-SUDS3. FIG. 20B shows the transcriptional repression of BRCA1, PSMD7, SEL1L, and ST3GAL4 by either synthetic or plasmid sgRNAs in A375 cells stably expressing dCas9-SALL1-SUDS3.

FIG. 21 shows the transcriptional repression of CD151, SEL1L, SETD3, and TFRC by either synthetic sgRNAs or synthetic crRNA:tracrRNA complexes in U2OS cells stably expressing dCas9-SALL1-SUDS3.

FIG. 22 shows the transcriptional repression of LBR, MRE11a, XRCC4, and SEL1L by synthetic sgRNAs with 5′ truncated 14 mer targeting regions or full length 20 mer targeting regions in U2OS cells stably expressing dCas9-SALL1-SUDS3.

FIG. 23A is a representation of the phenotypic effects of gene knockdown of PSMD7 and PSMD11 by synthetic sgRNAs containing various combinations of two 2′-O-methyl and phosphorothioate linkages (2×MS) and two locked nucleic acid (LNA) modifications at the 5′ and 3′ end of the sgRNA in U2OS Ubi[G76V]-EGFP reporter cells expressing dCas9-SALL1-SUDS3. FIG. 23B is a representation of the phenotypic effects of gene knockdown of PSMD7 and PSMD11 by synthetic sgRNAs end stabilized with two 2′-O-methyl and phosphorothioate linkages (2×MS) and containing various locked nucleic acids (LNA) at different positions in the targeting region in U2OS Ubi[G76V]-EGFP reporter cells expressing dCas9-SALL1-SUDS3.

FIG. 24A shows the transcriptional repression of BRCA1, CD151, and SETD3 by synthetic crRNA:tracrRNA complexes in which the tracrRNA contains an MS2 stem loop at various positions (in stem loop 2 or 3′ end of the tracrRNA) to recruit MCP-SALL1-SUDS3 to dCas9. FIG. 24B shows the transcriptional repression of BRCA1, CD151, and SETD3 by synthetic crRNA:tracrRNA complexes in which the tracrRNA contains various MS2 stem loop sequences to recruit MCP-SALL1-SUDS3 to dCas9.

FIG. 25A is a graph that shows the transcriptional repression and protein level knockdown of CXCR3 in primary human CD4+ T cells nucleofected with dCas9-SALL1-SUDS3 and either a synthetic non-targeting control or a pool of three guides targeting the gene of interest one and three days post-nucleofection. FIG. 25B provides representations of CXCR3 and CD4 protein expression in the aforementioned populations of T cells.

### DETAILED DESCRIPTION OF THE INVENTION

Reference will now be made in detail to various embodiments of the present invention, examples of which are illustrated in the accompanying figures. In the following description, numerous specific details are set forth in order to provide a thorough understanding of the present invention. However, unless otherwise indicated or implicit from context, the details are intended to be examples and should not be deemed to limit the scope of the invention in any way. Additionally, features described in connection with the various or specific embodiments are not to be construed as not appropriate for use in connection with other embodiments disclosed herein unless such exclusivity is explicitly stated or implicit from context.

Headers are provided herein for the convenience of the reader and do not limit the scope of any of the embodiments disclosed herein.

### Definitions

Unless otherwise stated or implicit from context the following terms and phrases have the meanings provided below.

The phrase “2′ modification” refers to a nucleotide unit having a sugar moiety that is modified at the 2′ position of the sugar moiety. An example of a 2′ modification is a 2′-O-alkyl modification that forms a 2′-O-alkyl modified nucleotide or a 2′ halogen modification that forms a 2′ halogen modified nucleotide.

The phrase “2′-O-alkyl modified nucleotide” refers to a nucleotide unit having a sugar moiety, for example, a deoxyribosyl or ribosyl moiety that is modified at the 2′ position such that an oxygen atom is attached both to the carbon atom located at the 2′ position of the sugar and to an alkyl group. In various embodiments, the alkyl moiety consists of or consists essentially of carbon(s) and hydrogens. When the O moiety and the alkyl group to which it is attached are viewed as one group, they may be referred to as an O-alkyl group, e.g., —O-methyl, —O-ethyl, —O-propyl, —O-isopropyl, —O-butyl, —O-isobutyl, —O-ethyl-O-methyl (—OCH₂CH₂OCH₃), and —O-ethyl-OH (—OCH₂CH₂OH). A 2′-O-alkyl modified nucleotide may be substituted or unsubstituted.

The phrase “2′ halogen modified nucleotide” refers to a nucleotide unit having a sugar moiety, for example, a deoxyribosyl moiety that is modified at the 2′ position such that the carbon at that position is directly attached to a halogen species, e.g., Fl, Cl, or Br.

The term “complementarity” refers to the ability of a nucleic acid to form one or more hydrogen bonds with another nucleic acid sequence by either traditional Watson-Crick base-pairing or other non-traditional types of base pairs. A percent complementarity indicates the percentage of residues in a nucleic acid molecule that can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary, respectively). “Perfect complementarity” means that all of the contiguous residues of a nucleic acid sequence will hydrogen bond with the same number of contiguous residues in a second nucleic acid sequence. “Substantially complementary” as used herein refers to a degree of complementarity that is at least 60%, at least 65%, at least 70%, at least 75%, at least 80%, at least 85%, at least 90%, at least 95%, at least 97%, at least 98%, or at least 99%, over a region of, for example, 8, 9, 10, 11, 12, 13, 14, 15, 16, 17, 18, 19, 20, 21, 22, 23, 24, 25, 30, 35, 40, 45, 50, or more consecutive nucleotides, or refers to two nucleic acids that hybridize under stringent conditions.

The term “encodes” refers to the ability of a nucleotide sequence or an amino acid sequence to provide information that describes the sequence of nucleotides or amino acids in another sequence or in a molecule. Thus, a nucleotide sequence encodes a molecule that contains the same nucleotides as in the nucleotide sequence that encodes it; that contains the complementary nucleotides according to Watson-Crick base pairing rules; that contains the RNA equivalent of the nucleotides that encode it; that contains the RNA equivalent of the complement of the nucleotides that encode it; that contains the amino acid sequence that can be generated based on the consecutive codons in the sequence; and that contains the amino acid sequence that can be generated based on the complement of the consecutive codons in the sequence.

A “gRNA” is a guide RNA. A gRNA comprises, consists essentially of, or consists of a CRISPR RNA (crRNA) and in some embodiments, it may also comprise a trans-activating CRISPR RNA (tracrRNA). It may be created synthetically or enzymatically, and it may be in the form of a contiguous strand of nucleotides in which case it is a “sgRNA” or in some embodiments, formed by the hybridization of a crRNA and a tracrRNA that are not covalently linked together to form a contiguous chain of nucleotides. Additionally, each gRNA (or component thereof, e.g., crRNA and tracrRNA if present) may independently be encoded by a plasmid, lentivirus, or AAV (adeno associated virus), a retrovirus, an adenovirus, a coronavirus, a Sendai virus or other vector. The gRNA introduces specificity into CRISPR/Cas systems. The specificity is dictated in part by base pairing between a target DNA and the sequence of a region of the gRNA that may be referred to as the spacer region or targeting region.

Another factor affecting specificity to gRNAs binding to a target DNA sequence is the presence of a PAM (protospacer-adjacent motif) sequence (also referred to as a PAM site) in a target sequence. Each target sequence and its corresponding PAM site/sequence may collectively be referred to as a Cas-targeted site. For example, the Class 2 CRISPR system of S. pyogenes uses targeted sites having N12-20NGG, where NGG represents the PAM site from S. pyogenes, and N12-20 represents the 12-20 nucleotides directly 5′ to the PAM site. Additional PAM site sequences from other species of bacteria include NGGNG, NNNNGATT, NNAGAA, NNAGAAW, and NAAAAC. See, e.g., US 20140273233, WO 2013176772, Cong et al., Science 339 (6121): 819-823 (2012), Jinek et al., Science 337 (6096): p. 816-821 (2012), Mali et al., Science 339 (6121): p. 823-826 (2013), Gasiunas et al., Proc Natl Acad. Sci. USA, 109 (39): p. E2579-E2586 (2012), Cho et al., Nature Biotechnology 31: p. 230-232 (2013), Hou et al., Proc. Natl Acad. Sci. U SA. 110(39): p. 15644-15649 (2013), Mojica et al., Microbiology 155 (Pt 3): p. 733-740 (2009), and www.addgene.org/CRISPR/. The contents of these documents are incorporated herein by reference in their entireties.

The terms “hybridization” and “hybridizing” refer to a process in which completely, substantially, or partially complementary nucleic acid strands come together under specified hybridization conditions to form a double-stranded structure or region in which the two constituent strands are joined by hydrogen bonds. Unless otherwise stated, the hybridization conditions are naturally occurring or lab-designed conditions. Although hydrogen bonds typically form between adenine and thymine or uracil (A and T or U) or between cytidine and guanine (C and G), other base pairs may form (see e.g., Adams et al., The Biochemistry of the Nucleic Acids, 11th ed., 1992).

A “ligand binding moiety” refers to a moiety such as an aptamer e.g., oligonucleotide or peptide or another compound that binds to a specific ligand and can reversibly or irreversibly be associated with that ligand. To be reversibly associated means that two molecules or complexes can retain association with each other by, for example, noncovalent forces such as hydrogen bonding, and be separated from each other without either molecule or complex losing the ability to associate with other molecules or complexes.

The term “modified nucleotide” refers to a nucleotide having at least one modification in the chemical structure of the base, sugar and/or phosphate, including, but not limited to, 5-position pyrimidine modifications, 8-position purine modifications, modifications at cytosine exocyclic amines, and substitution of 5-bromo-uracil or 5-iodouracil; and 2′-modifications, including but not limited to, sugar-modified ribonucleotides in which the 2′-OH is replaced by a group such as an H, OR, R, halo, SH, SR, NH₂, NHR, NR₂, or CN.

Modified bases refer to nucleotide bases such as, for example, adenine, guanine, cytosine, thymine, uracil, xanthine, inosine, and queuosine that have been modified by the replacement or addition of one or more atoms or groups. Some examples of these types of modifications include, but are not limited to, alkylated, halogenated, thiolated, aminated, amidated, or acetylated bases, alone and in various combinations. More specific modified bases include, for example, 5-propynyluridine, 5-propynylcytidine, 6-methyladenine, 6-methylguanine, N,N,-dimethyladenine, 2-propyladenine, 2-propylguanine, 2-aminoadenine, 1-methylinosine, 3-methyluridine, 5-methylcytidine, 5-methyluridine and other nucleotides having a modification at the position, 5-(2-amino)propyluridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2,2-dimethylguanosine, 5-methylaminoethyluridine, 5-methyloxyuridine, deazanucleotides such as 7-deaza-adenosine, 6-azouridine, 6-azocytidine, 6-azothymidine, 5-methyl-2-thiouridine, other thio bases such as 2-thiouridine and 4-thiouridine and 2-thiocytidine, dihydrouridine, pseudouridine, queuosine, archaeosine, naphthyl and substituted naphthyl groups, any O- and N-alkylated purines and pyrimidines such as N6-methyladenosine, 5-methylcarbonylmethyluridine, uridine 5-oxyacetic acid, pyridine-4-one, pyridine-2-one, phenyl and modified phenyl groups such as aminophenol or 2,4,6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyl nucleotides, and alkylcarbonylalkylated nucleotides. Modified nucleotides also include those nucleotides that are modified with respect to the sugar moiety, as well as nucleotides having sugars or analogs thereof that are not ribosyl. For example, the sugar moieties may be, or be based on, mannoses, arabinoses, glucopyranoses, galactopyranoses, 4-thioribose, and other sugars, heterocycles, or carbocycles.

The term “nucleotide” refers to a ribonucleotide or a deoxyribonucleotide or modified form thereof, as well as an analog thereof. Nucleotides include species that comprise purines, e.g., adenine, hypoxanthine, guanine, and their derivatives and analogs, as well as pyrimidines, e.g., cytosine, uracil, thymine, and their derivatives and analogs. Preferably, a nucleotide comprises a cytosine, uracil, thymine, adenine, or guanine moiety. Further, the term nucleotide also includes those species that have a detectable label, such as for example a radioactive or fluorescent moiety, or mass label attached to the nucleotide. The term nucleotide also includes what are known in the art as universal bases. By way of example, universal bases include but are not limited to 3-nitropyrrole, 5-nitroindole, or nebularine. Nucleotide analogs are, for example, meant to include nucleotides with bases such as inosine, queuosine, xanthine, sugars such as 2′-methyl ribose, and non-natural phosphodiester internucleotide linkages such as methylphosphonates, phosphorothioates, phosphoroacetates and peptides.

The term “repressor domain” refers to the amino acid sequence that form the domain of a repressor molecule that leads to inhibition of the expression of a gene.

The terms “subject” and “patient” are used interchangeably herein to refer to an organism, e.g., a vertebrate, preferably a mammal, more preferably a human. Mammals include, but are not limited to, murines, simians, humans, farm animals, sport animals, and pets such as dogs and cats. The tissues, cells and their progeny of an organism or other biological entity obtained in vivo or cultured in vitro are also encompassed within the terms subject and patient. Additionally, in some embodiments, a subject may be an invertebrate animal, for example, an insect or a nematode; while in others, a subject may be a plant or a fungus.

A “terminal amino acid” is the last amino acid within a protein or within a region of a fusion protein. Within a fusion protein a terminal amino acid of a Cas protein may, for example, be bound not only to another amino acid within the Cas protein region of the fusion protein, but also to a repressor domain or to a linker. Similarly, within a fusion protein, a terminal amino acid of a repressor domain may, for example, be bound not only to another amino acid within the repressor domain, but also to another repressor domain or to a Cas protein region of a fusion protein or to a linker. A terminal amino acid may be a C terminal amino acid or an N terminal amino acid.

As used herein, “treatment,” “treating,” “palliating,” and “ameliorating” are used interchangeably. These terms refer to an approach for obtaining beneficial or desired results including, but not limited to, a therapeutic benefit and/or a prophylactic benefit. By therapeutic benefit is meant any therapeutically relevant improvement in or effect on one or more diseases, conditions, or symptoms under treatment. For prophylactic benefit, the complexes of the present invention may be administered to a subject, or a subject's cells or tissues, or those of another subject extracorporeally before re-administration, at risk of developing a particular disease, condition, or symptom, or to a subject reporting one or more of the physiological symptoms of a disease, even though the disease, condition, or symptom might not have yet been manifested.

The term “vector” refers to a molecule or complex that transports another molecule and includes but is not limited to a nucleic acid molecule capable of transporting another nucleic acid molecule to which it has been linked, or that has been incorporated within the vector sequence. A vector can be introduced into cells and organisms to express RNA transcripts, proteins, and peptides, and may be termed an “expression vector.” Examples of vectors include, but are not limited to, plasmids, lentiviruses, alphaviruses, adenoviruses, or adeno-associated viruses. The vector may be single stranded, double stranded or have at least one region that is single stranded and at least one region that is double stranded. Further, the nucleic acid may comprise, consist essentially of, or consist of RNA or DNA.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention.

The term “about” generally refers to plus or minus 10% of the indicated number. For example, “about 10%” may indicate a range of 9% to 11%, and “about 20” may mean from 18-22. Other meanings of “about” may be apparent from the context, such as rounding off; for example “about 1” may also mean from 0.5 to 1.4.

Various embodiments of the present invention are directed to fusion proteins and their uses. Fusion proteins are molecules that contain a portion or a complete amino sequence of each of two or more proteins. The components of fusion proteins may be fused directly to each other through, for example, covalent bonds or through linkers as described below. Fusion proteins may also be associated with moieties that are do not contain amino acids such as nucleotides sequences.

### Cas Fusion Proteins

According to a first embodiment, the present invention is directed to a Cas fusion protein. A Cas fusion protein comprises, consists essentially of, or consists of a Cas protein and one or both of a SALL1 repressor domain and a SUDS3 repressor domain or a sequence that is at least 80%, at least 85%, at least 90%, or at least 95% the same as one of the aforementioned repressor domains.

The Cas protein may be any CRISPR associated protein that is naturally occurring in for example, archaea or bacteria, or a modified version thereof such as a deactivated version, a truncated version thereof, or a derivative thereof. Amino acid sequences and nucleic acids sequences for numerous Cas proteins are available through publicly available sources such as the United States of America's National Institute of Health: https://www.ncbi.nim.nlh.gov/or Uniprot https://www.uniprot.org/the entire contents of which are incorporated by reference herein.

Examples of Cas proteins include but are not limited to: Cas1, Cas1B, Cas2, Cas3, Cas4, Cas5, Cas5e (CasD), Cas6, Cas6e, Cas6f, Cas7, Cas8al, Cas8a2, Cas8b, Cas8c, Cas9 (Csn1 or Csx12), CasIO, CaslOd, Cas12a, Cas12b, Cas12c, Cas12d, Cas12e, Cas12f, Cas12h, Cas12i, Cas12j, Mad7, CasX, CasY, Cas 13a, Cas14, C2cl, C2c2, C2c3, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, CsxIO, Csx16, CsaX, Csx3, Csx1, Csx15, Csf1, Csf2, Csf3, Csf4, and Cul966, and homologs or modified versions thereof. Unless otherwise stated or implicit from context the recitation of a Cas protein includes all active and deactivated versions, as well as homologs and derivatives thereof.

In some embodiments, the Cas protein is a Type II Cas protein such as Cas9 or a Type V Cas protein such as Cas12a, Cas12b, Cas12c, Cas12d, Cas12e, Cas12f, Cas12h, Cas12i, Cas12j, and MAD7.

Modified versions of Cas proteins that may be used in the present invention, include but are not limited to catalytically inactive versions such as dCas9 and dCas12 or versions that have modified attenuated catalytic activity to provide a nicking function such as the nickase nCas9. A nicking enzyme is an enzyme that cuts one strand of a double-stranded DNA at a specific recognition nucleotide sequence. These enzymes cut only one strand of the DNA duplex, to produce DNA molecules that are “nicked,” rather than cleaved. Examples of amino acid sequences of Cas proteins that may be of use in connection with the present invention are:

Deactivated Cas9:

(SEQ ID NO: 182)
MDYKDDDDKMAPKKKRKVGIHGVPAADKKYSIGLAIGTNSVGWAVITDEY
KVPSKKFKVLGNTDRHSIKKNLIGALLFDSGETAEATRLKRTARRRYTRR
KNRICYLQEIFSNEMAKVDDSFFHRLEESFLVEEDKKHERHPIFGNIVDE
VAYHEKYPTIYHLRKKLVDSTDKADLRLIYLALAHMIKFRGHFLIEGDLN
PDNSDVDKLFIQLVQTYNQLFEENPINASGVDAKAILSARLSKSRRLENL
IAQLPGEKKNGLFGNLIALSLGLTPNFKSNFDLAEDAKLQLSKDTYDDDL
DNLLAQIGDQYADLFLAAKNLSDAILLSDILRVNTEITKAPLSASMIKRY
DEHHQDLTLLKALVRQQLPEKYKEIFFDQSKNGYAGYIDGGASQEEFYKF
IKPILEKMDGTEELLVKLNREDLLRKQRTFDNGSIPHQIHLGELHAILRR
QEDFYPFLKDNREKIEKILTFRIPYYVGPLARGNSRFAWMTRKSEETITP
WNFEEVVDKGASAQSFIERMTNFDKNLPNEKVLPKHSLLYEYFTVYNELT
KVKYVTEGMRKPAFLSGEQKKAIVDLLFKTNRKVTVKQLKEDYFKKIECF
DSVEISGVEDRFNASLGTYHDLLKIIKDKDFLDNEENEDILEDIVLTLTL
FEDREMIEERLKTYAHLFDDKVMKQLKRRRYTGWGRLSRKLINGIRDKQS
GKTILDFLKSDGFANRNFMQLIHDDSLTFKEDIQKAQVSGQGDSLHEHIA
NLAGSPAIKKGILQTVKVVDELVKVMGRHKPENIVIEMARENQTTQKGQK
NSRERMKRIEEGIKELGSQILKEHPVENTQLQNEKLYLYYLQNGRDMYVD
QELDINRLSDYDVDAIVPQSFLKDDSIDNKVLTRSDKNRGKSDNVPSEEV
VKKMKNYWRQLLNAKLITQRKFDNLTKAERGGLSELDKAGFIKRQLVETR
QITKHVAQILDSRMNTKYDENDKLIREVKVITLKSKLVSDFRKDFQFYKV
REINNYHHAHDAYLNAVVGTALIKKYPKLESEFVYGDYKVYDVRKMIAKS
EQEIGKATAKYFFYSNIMNFFKTEITLANGEIRKRPLIETNGETGEIVWD
KGRDFATVRKVLSMPQVNIVKKTEVQTGGFSKESILPKRNSDKLIARKKD
WDPKKYGGFDSPTVAYSVLVVAKVEKGKSKKLKSVKELLGITIMERSSFE
KNPIDFLEAKGYKEVKKDLIIKLPKYSLFELENGRKRMLASAGELQKGNE
LALPSKYVNFLYLASHYEKLKGSPEDNEQKQLFVEQHKHYLDEIIEQISE
FSKRVILADANLDKVLSAYNKHRDKPIREQAENIIHLFTLTNLGAPAAFK
YFDTTIDRKRYTSTKEVLDATLIHQSITGLYETRIDLSQLGGDKRPAATK
KAGQAKKKK

Deactivated MAD7:

(SEQ ID NO: 39)
MVDGKPIPNPLLGLDSTPKKKRKVNNGTNNFQNFIGISSLQKTLRNALIP
TETTQQFIVKNGIIKEDELRGENRQILKDIMDDYYRGFISETLSSIDDID
WTSLFEKMEIQLKNGDNKDTLIKEQTEYRKAIHKKFANDDRFKNMFSAKL
ISDILPEFVIHNNNYSASEKEEKTQVIKLESRFATSFKDYFKNRANCFSA
DDISSSSCHRIVNDNAEIFFSNALVYRRIVKSLSNDDINKISGDMKDSLK
EMSLEEIYSYEKYGEFITQEGISFYNDICGKVNSFMNLYCQKNKENKNLY
KLQKLHKQILCIADTSYEVPYKFESDEEVYQSVNGFLDNISSKHIVERLR
KIGDNYNGYNLDKIYIVSKFYESVSQKTYRDWETINTALEIHYNNILPGN
GKSKADKVKKAVKNDLQKSITEINELVSNYKLCSDDNIKAETYIHEISHI
LNNFEAQELKYNPEIHLVESELKASELKNVLDVIMNAFHWCSVFMTEELV
DKDNNFYAELEEIYDEIYPVISLYNLVRNYVTQKPYSTKKIKLNFGIPTL
ADGWSKSKEYSNNAIILMRDNLYYLGIFNAKNKPDKKIIEGNTSENKGDY
KKMIYNLLPGPNKMIPKVFLSSKTGVETYKPSAYILEGYKQNKHIKSSKD
FDITFCHDLIDYFKNCIAIHPEWKNFGFDFSDTSTYEDISGFYREVELQG
YKIDWTYISEKDIDLLQEKGQLYLFQIYNKDFSKKSTGNDNLHTMYLKNL
ESEENLKDIVLKLNGEAEIFFRKSSIKNPIIHKKGSILVNRTYEAEEKDQ
FGNIQIVRKNIPENIYQELYKYFNDKSDKELSDEAAKLKNVVGHHEAATN
IVKDYRYTYDKYFLHMPITINFKANKTGFINDRILQYIAKEKDLHVIGIA
RGERNLIYVSVIDTCGNIVEQKSFNIVNGYDYQIKLKQQEGARQIARKEW
KEIGKIKEIKEGYLSLVIHEISKMVIKYNAIIAMADLSYGFKKGRFKVER
QVYQKFETMLINKLNYLVFKDISITENGGLLKGYQLTYIPDKLKNVGHQC
GCIFYVPAAYTSKIDPTTGFVNIFKFKDLTVDAKREFIKKFDSIRYDSEK
NLFCFTFDYNNFITQNTVMSKSSWSVYTYGVRIKRRFVNGRFSNESDTID
ITKDMEKTLEMTDINWRDGHDLRQDIIDYEIVQHIFEIFRLTVQMRNSLS
ELEDRDYDRLISPVLNENNIFYDSAKAGDALPKDAAANGAYCIALKGLYE
IKQITENWKEDGKFSRDKLKISNKDWFDFIQNKRYLKRPAATKKAGQAKK
KK

Deactivated CasPhi8 (dCasPhi8):

(SEQ ID NO: 40)
MVDGSGPAAKRVKLDSGGIKPTVSQFLTPGFKLIRNHSRTAGLKLKNEGE
EACKKFVRENEIPKDECPNFQGGPAIANIIAKSREFTEWEIYQSSLAIQE
VIFTLPKDKLPEPILKEEWRAQWLSEHGLDTVPYKEAAGLNLIIKNAVNT
YKGVQVKVDNKNKNNLAKINRKNEIAKLNGEQEISFEEIKAFDDKGYLLQ
KPSPNKSIYCYQSVSPKPFITSKYHNVNLPEEYIGYYRKSNEPIVSPYQF
DRLRIPIGEPGYVPKWQYTFLSKKENKRRKLSKRIKNVSPILGIICIKKD
WCVFDMRGLLRTNHWKKYHKPTDSINDLFDYFTGDPVIDTKANVVRFRYK
MENGIVNYKPVREKKGKELLENICDQNGSCKLATVAVGONNPVAIGLFEL
KKVNGELTKTLISRHPTPIDFCNKITAYRERYDKLESSIKLDAIKQLTSE
QKIEVDNYNNNFTPQNTKQIVCSKLNINPNDLPWDKMISGTHFISEKAQV
SNKSEIYFTSTDKGKTKDVMKSDYKWFQDYKPKLSKEVRDALSDIEWRLR
RESLEFNKLSKSREQDARQLANWISSMCDVIGIENLVKKNNFFGGSGKRE
PGWDNFYKPKKENRWWINAIHKALTELSQNKGKRVILLPAMRTSITCPKC
KYCDSKNRNGEKFNCLKCGIELNADIDVATENLATVAITAQSMPKPTCER
SGDAKKPVRARKAKAPEFHDKLAPSYTVVLREAVKRPAATKKAGQAKKKK

The Cas proteins may be used with repressor domains. The repressor domain of SALL1 is:

(SEQ ID NO: 1)
MSRRKQAKPQHFQSDPEVASLPRRDGDTEKGQPSRPTKSKDAHVCGRCCA
EFFELSDLLLHKKNCTKNQLVLIVNENPASPPETFSPSPPPDNPDEQMND
TVNKTDQVDCSDLSEHNGLDREESMEVEAPVANKSGSGTSSGSHSSTAPS
SSSSSSSSSGGGGSSSTGTSAITTSLPQLGDLT.

The repressor domain of SUDS3 is:

(SEQ ID NO: 2)
MSAAGLLAPAPAQAGAPPAPEYYPEEDEELESAEDDERSCRGRESDEDTE
DASETDLAKHDEEDYVEMKEQMYQDKLASLKRQLQQLQEGTLQEYQKRMK
KLDQQYKERIRNAELFLQLETEQVERNYIKEKKAAVKEFEDKKVELKENL
IAELEEKKKMIENEKLTMELTGDSMEVKPIMTRKLRRRPNDPVPIPDKRR
KPAPAQLNYLLTDEQIMEDLRTLNKLKSPKRPASPSSPEHLPATPAESPA
QRFEARIEDGKLYYDKRWYHKSQAIYLESKDNQKLSCVISSVGANEIWVR
KTSDSTKMRIYLGQLQRGLFVIRRRSAA.

In some embodiments, the Cas fusion protein comprises, consists essentially of, or consists of a Cas protein and the SALL1 repressor domain or a repressor domain that is at least 80%, at least 85%, at least 90%, or at least 95% similar to SEQ ID NO: 1. In some embodiments, the SALL1 repressor domain or a repressor domain that is at least 80%, at least 85%, at least 90%, or at least 95% similar to SEQ ID NO: 1 is attached to the N terminal amino acid of the Cas protein. In some embodiments, the SALL1 repressor domain or a repressor domain that is at least 80%, at least 85%, at least 90%, or at least 95% similar to SEQ ID NO: 1 is attached to the C terminal amino acid of the Cas protein.

In some embodiments, the Cas fusion protein comprises, consists essentially of, or consists of a Cas protein and the SUDS3 repressor domain or a repressor domain that is at least 80%, at least 85%, at least 90%, or at least 95% similar to SEQ ID NO: 2. In some embodiments, the SUDS3 repressor domain or a repressor domain that is at least 80%, at least 85%, at least 90%, or at least 95% similar to SEQ ID NO: 2 is attached to the N terminal amino acid of the Cas protein. In some embodiments, the SUDS3 repressor domain or a repressor domain that is at least 80%, at least 85%, at least 90%, or at least 95% similar to SEQ ID NO: 2 is attached to the C terminal amino acid of the Cas protein.

In some embodiments, the Cas fusion protein comprises, consists essentially of, or consists of a Cas protein and both the SALL1 repressor domain and the SUDS3 repressor domain. In some embodiments, this Cas fusion protein is organized in one of the following ways (written N terminus to C terminus):
[Cas protein]-[SALL1 repressor domain]-[SUDS3 repressor domain]
[Cas protein]-[SUDS3 repressor domain]-[SALL1 repressor domain]
[SALL1 repressor domain]-[SUDS3 repressor domain]-[Cas protein]
[SUDS3 repressor domain]-[SALL1 repressor domain]-[Cas protein]
[SALL1 repressor domain]-[Cas protein]-[SUDS3 repressor domain]
[SUDS3 repressor domain]-[Cas protein]-[SALL1 repressor domain]

In some embodiments, the Cas fusion protein comprises a SALL1 repressor domain and a SUDS3 repressor domain, wherein the SALL1 repressor domain comprises, consists essentially of, or consists of a sequence that is at least 80%, at least 85%, at least 90%, or at least 95% similar to SEQ ID NO: 1 and the SUDS3 repressor domain comprises, consists essentially of, or consists of a sequence that is at least 80%, at least 85%, at least 90%, or at least 95% similar to SEQ ID NO: 2. In some embodiments, the Cas fusion protein comprises a SALL1 repressor domain and a SUDS3 repressor domain, wherein the SALL1 repressor domain comprises, consists essentially of, or consists of a sequence is the same as SEQ ID NO: 1 and the SUDS3 repressor domain comprises, consists essentially of, or consists of a sequence that is the same as SEQ ID NO: 2.

In some embodiments, the Cas fusion protein comprises, consists essentially of, or consists of a Cas protein and two or more copies of both the SALL1 repressor domain and the SUDS3 repressor domain. In some embodiments, this Cas fusion protein is organized in one of the following ways:
[SALL1 repressor domain]-[SUDS3 repressor domain]-[Cas protein]-[SALL1 repressor domain]-[SUDS3 repressor domain]
[SALL1 repressor domain]-[SUDS3 repressor domain]-[Cas protein]-[SUDS3 repressor domain]-[SALL1 repressor domain]
[SUDS3 repressor domain]-[SALL1 repressor domain]-[Cas protein]-[SALL1 repressor domain]-[SUDS3 repressor domain]
[SUDS3 repressor domain]-[SALL1 repressor domain]-[Cas protein]-[SUDS3 repressor domain]-[SALL1 repressor domain]

In some embodiments, the Cas fusion protein comprises a plurality of SALL1 repressor domains and a plurality of SUDS3 repressor domains, wherein each SALL1 repressor domain comprises, consists essentially of, or consists of a sequence that is at least 80%, at least 85%, at least 90%, or at least 95% similar to SEQ ID NO: 1 and each SUDS3 repressor domain comprises, consists essentially of, or consists of a sequence that is at least 80%, at least 85%, at least 90%, or at least 95% similar to SEQ ID NO: 2. In some embodiments, the Cas fusion protein comprises a plurality of SALL1 repressor domains and a plurality of SUDS3 repressor domains, wherein each SALL1 repressor domain comprises, consists essentially of, or consists of a sequence is the same as SEQ ID NO: 1 and each SUDS3 repressor domain comprises, consists essentially of, or consists of a sequence that is the same as SEQ ID NO: 2.

In some embodiments, the Cas fusion protein also comprises a domain of an additional repressor protein: [R]. In some embodiments, [R] is selected from the group consisting of the NIPP1 repressor domain, the KRAB repressor domain, the DNMT3A repressor domain, the BCL6 repressor domain, the CbpA repressor domain, the H-NS repressor domain, the MBD3 repressor domain, and the KRAB-Me-CP2 repressor domain.

The NIPP1 repressor domain, may be represented as follows:

(SEQ ID NO: 34)
MVQTAVVPVKKKRVEGPGSLGLEESGSRRMQNFAFSGGLYGGLPPTHSEA
GSQPHGIHGTALIGGLPMPYPNLAPDVDLTPVVPSAVNMNPAPNPAVYNP
EAVNEPKKKKYAKEAWPGKKPTPSLLI

or a sequence that is at least 80%, at least 85%, at least 90%, or at least 95% that same as SEQ ID NO: 34.

The KRAB repressor domain, may be represented as follows:

(SEQ ID NO: 35)
MDAKSLTAWSRTLVTFKDVFVDFTREEWKLLDTAQQIVYRNVMLENYKNL
VSLGYQLTKPDVILRLEKGEEPWLV

or a sequence that is at least 80%, at least 85%, at least 90%, or at least 95% that same as SEQ ID NO: 35.

The DNMT3A repressor domain, may be represented as follows:

(SEQ ID NO: 36)
PSRLQMFFANNHDQEFDPPKVYPPVPAEKRKPIRVLSLFDGIATGLLVLK
DLGIQVDRYIASEVCEDSITVGMVRHQGKIMYVGDVRSVTQKHIQEWGPF
DLVIGGSPCNDLSIVNPARKGLYEGTGRLFFEFYRLLHDARPKEGDDRPF
FWLFENVVAMGVSDKRDISRFLESNPVMIDAKEVSAAHRARYFWGNLPGM
NRPLASTVNDKLELQECLEHGRIAKFSKVRTITTRSNSIKQGKDQHFPVF
MNEKEDILWCTEMERVFGFPVHYTDVSNMSRLARQRLLGRSWSVPVIRHL
FAPLKEYFACV

or a sequence that is at least 80%, at least 85%, at least 90%, or at least 95% that same as SEQ ID NO: 36.

The BCL6 repressor domain may be represented as follows:

(SEQ ID: 173)
MASPADSCIQFTRHASDVLLNLNRLRSRDILTDVVIVVSREQFRAHKTVL
MACSGLFYSIFTDQLKCNLSVINLDPEINPEGFCILLDFMYTSRLNLREG
NIMAVMATAMYLQMEHVVDTCRKFIKASEAEM

or a sequence that is at least 80%, at least 85%, at least 90%, or at least 95% that same as SEQ ID NO: 173.

The CbpA repressor domain may be represented as follows:

(SEQ ID: 174)
MELKDYYAIMGVKPTDDLKTIKTAYRRLARKYHPDVSKEPDAEARFKEVA
EAWEVLSDEQRRAEYDQMWQHRNDPQFNRQFHHGDGQSFNAEDFDDIFSS
IFGQHARQSRQRPATRGHDIEIEVAVFLEETLTEHKRTISYNLPVYNAFG
MIEQEIPKTLNVKIPAGVGNGQRIRLKGQGTPGENGGPNGDLWLVIHIAP
HPLFDIVGQDLEIVVPVSPWEAALGAKVTVPTLKESILLTIPPGSQAGQR
LRVKGKGLVSKKQTGDLYAVLKIVMPPKPDENTAALWQQLADAOSSFDPR
KDWGKA

or a sequence that is at least 80%, at least 85%, at least 90%, or at least 95% that same as SEQ ID NO: 174.

The H-NS repressor domain may be represented as follows:

(SEQ ID: 175)
MSEALKILNNIRTLRAQARECTLETLEEMLEKLEVVVNERREEESAAAAE
VEERTRKLQQYREMLIADGIDPNELLNSLAAVKSGTKAKRAQRPAKYSYV
DENGETKTWTGQGRTPAVIKKAMDEQGKSLDDFLIKQ

or a sequence that is at least 80%, at least 85%, at least 90%, or at least 95% that same as SEQ ID NO: 175.

The MBD3 repressor domain may be represented as follows:

(SEQ ID: 176)
MERKRWECPALPQGWEREEVPRRSGLSAGHRDVFYYSPSGKKFRSKPQLA
RYLGGSMDLSTFDFRTGKMLMSKMNKSRQRVRYDSSNQVKGKPDLNTALP
VRQTASIFKQPVTKITNHPSNKVKSDPQKAVDQPRQLFWEKKLSGLNAFD
IAEELVKTMDLPKGLQGVGPGCTDETLLSAIASALHTSTMPITGOLSAAV
EKNPGVWLNTTQPLCKAFMVTDEDIRKQEELVQQVRKRLEEALMADMLAH
VEELARDGEAPLDKACAEDDDEEDEEEEEEEPDPDPEMEHV

or a sequence that is at least 80%, at least 85%, at least 90%, or at least 95% that same as SEQ ID NO: 176.

The KRAB-MeCP2 repressor domain may be represented as follows:

(SEQ ID: 177)
MDAKSLTAWSRTLVTFKDVFVDFTREEWKLLDTAQQIVYRNVMLENYKNL
VSLGYQLTKPDVILRLEKGEEPWLVSGGGSGGSGSSPKKKRKVEASVQVK
RVLEKSPGKLLVKMPFQASPGGKGEGGGATTSAQVMVIKRPGRKRKAEAD
PQAIPKKRGRKPGSVVAAAAAEAKKKAVKESSIRSVQETVLPIKKRKTRE
TVSIEVKEVVKPLLVSTLGEKSGKGLKTCKSPGRKSKESSPKGRSSSASS
PPKKE

or a sequence that is at least 80%, at least 85%, at least 90%, or at least 95% that same as SEQ ID NO: 177.

Examples of the orientation of these sequences may be represented as follows:
[Cas protein]-[SALL1 repressor domain]-[R]
[Cas protein]-[SUDS3 repressor domain]-[R]
[R]-[SUDS3 repressor domain]-[Cas protein]
[R]-[SALL1 repressor domain]-[Cas protein]
[Cas protein]-[R]-[SUDS3 repressor domain]
[Cas protein]-[R]-[SALL1 repressor domain]
[SALL1 repressor domain]-[R] [Cas protein]
[SUDS3 repressor domain]-[R]-[Cas protein]
[R]-[Cas protein]-[SUDS3 repressor domain]
[R]-[Cas protein]-[SALL1 repressor domain]
[SALL1 repressor domain]-[Cas protein]-[R]
[SUDS3 repressor domain]-[Cas protein]-[R]

Further, in some embodiments, the Cas fusion protein comprises, consists essentially of, or consists of a Cas protein and each of the SALL1 repressor domain, the SUDS3 repressor domain, and the [R] repressor domain. When all three repressor domains are present, they may all be on the C terminal amino acid of the Cas protein, all be on the N terminal amino acid of the Cas protein, two be on the C terminal amino acid of the Cas protein and one be on the N terminal amino acid of the Cas protein, or two be on the N terminal amino acid of the Cas protein and one be on the C terminal amino acid of the Cas protein. Examples of the orientation of these sequences may be represented as follows:
[Cas protein]-[SALL1 repressor domain]-[R]-[SUDS3 repressor domain]
[Cas protein]-[SALL1 repressor domain]-[SUDS3 repressor domain]-[R]
[Cas protein]-[SUDS3 repressor domain]-[SALL1 repressor domain]-[R]
[Cas protein]-[SUDS3 repressor domain]-[R]-[SALL1 repressor domain]
[Cas protein]-[R]-[SUDS3 repressor domain]-[SALL1 repressor domain]
[Cas protein]-[R]-[SALL1 repressor domain]-[SUDS3 repressor domain]
[SALL1 repressor domain]-[R]-[SUDS3 repressor domain]-[Cas protein]
[SALL1 repressor domain]-[SUDS3 repressor domain]-[R]-[Cas protein]
[SUDS3 repressor domain]-[SALL1 repressor domain]-[R]-[Cas protein]
[SUDS3 repressor domain]-[R]-[SALL1 repressor domain]-[Cas protein]
[R]-[SUDS3 repressor domain]-[SALL1 repressor domain]-[Cas protein]
[R]-[SALL1 repressor domain]-[SUDS3 repressor domain]-[Cas protein]
[SALL1 repressor domain]-[Cas protein]-[R]-[SUDS3 repressor domain]
[SALL1 repressor domain]-[Cas protein]-[SUDS3 repressor domain]-[R]
[SUDS3 repressor domain]-[Cas protein]-[SALL1 repressor domain]-[R]
[SUDS3 repressor domain]-[Cas protein]-[R]-[SALL1 repressor domain]
[R]-[Cas protein]-[SUDS3 repressor domain]-[SALL1 repressor domain]
[R]-[Cas protein]-[SALL1 repressor domain]-[SUDS3 repressor domain]
[R]-[SUDS3 repressor domain]-[Cas protein]-[SALL1 repressor domain]
[SUDS3 repressor domain]-[R]-[Cas protein]-[SALL1 repressor domain]
[SALL1 repressor domain]-[R]-[Cas protein]-[SUDS3 repressor domain]
[R]-[SALL1 repressor domain]-[Cas protein]-[SUDS3 repressor domain]
[SUDS3 repressor domain]-[SALL1 repressor domain]-[Cas protein]-[R]
[SALL1 repressor domain]-[SUDS3 repressor domain]-[Cas protein]-[R]

By way of a non-limiting example, in some embodiments, in the Cas fusion protein the Cas protein is dCas9 or dCas12 such as dCas12a and the Cas fusion protein comprises, consists essentially of or consists of both the SALL1 repressor domain and the SUDS3 repressor domain.

Examples of amino acid sequences of fusion constructs of the present invention, include but are not limited to:

MCP-SALL1-SUDS3 amino acid sequence:

(SEQ ID NO: 41)
MASNFTQFVLVDNGGTGDVTVAPSNFANGVAEWISSNSRSQAYKVTCSVR
QSSAQKRKYTIKVEVPKVATQTVGGVELPVAAWRSYLNMELTIPIFATNS
DCELIVKAMQGLLKDGNPIPSAIAANSGIYSAGGGGSGGGGSGGGGSGPK
KKRKVAAAGSMSRRKQAKPQHFQSDPEVASLPRRDGDTEKGQPSRPTKSK
DAHVCGRCCAEFFELSDLLLHKKNCTKNQLVLIVNENPASPPETFSPSPP
PDNPDEQMNDTVNKTDQVDCSDLSEHNGLDREESMEVEAPVANKSGSGTS
SGSHSSTAPSSSSSSSSSSGGGGSSSTGTSAITTSLPQLGDLTGSGGGSG
GSGSMSAAGLLAPAPAQAGAPPAPEYYPEEDEELESAEDDERSCRGRESD
EDTEDASETDLAKHDEEDYVEMKEQMYQDKLASLKRQLQQLQEGTLQEYQ
KRMKKLDQQYKERIRNAELFLQLETEQVERNYIKEKKAAVKEFEDKKVEL
KENLIAELEEKKKMIENEKLTMELTGDSMEVKPIMTRKLRRRPNDPVPIP
DKRRKPAPAQLNYLLTDEQIMEDLRTLNKLKSPKRPASPSSPEHLPATPA
ESPAQRFEARIEDGKLYYDKRWYHKSQAIYLESKDNQKLSCVISSVGANE
IWVRKTSDSTKMRIYLGQLQRGLFVIRRRSAA

SEQ ID NO: 41 may, for example be coded by nucleic acid comprises, consisting essentially of or consisting of SEQ ID NO: 170

ATGGCTTCAAACTTTACTCAGTTCGTGCTCGTGGACAATGGTGGGACAGG
GGATGTGACAGTGGCTCCTTCTAATTTCGCTAATGGGGTGGCAGAGTGGA
TCAGCTCCAACTCACGGAGCCAGGCCTACAAGGTGACATGCAGCGTCAGG
CAGTCTAGTGCCCAGAAGAGAAAGTATACCATCAAGGTGGAGGTCCCCAA
AGTGGCTACCCAGACAGTGGGCGGAGTCGAACTGCCTGTCGCCGCTTGGA
GGTCCTACCTGAACATGGAGCTCACTATCCCAATTTTCGCTACCAATTCT
GACTGTGAACTCATCGTGAAGGCAATGCAGGGGCTCCTCAAAGACGGTAA
TCCTATCCCTTCCGCCATCGCCGCTAACTCAGGTATCTACAGCGCTGGAG
GAGGTGGAAGCGGAGGAGGAGGAAGCGGAGGAGGAGGTAGCGGACCTAAG
AAAAAGAGGAAGGTGGCGGCCGCTGGATCCATGAGTAGGAGAAAACAAGC
AAAACCACAGCACTTTCAAAGTGATCCTGAGGTAGCAAGCCTTCCACGGC
GGGACGGTGACACGGAGAAGGGTCAACCAAGTCGACCCACGAAAAGCAAA
GATGCTCATGTATGTGGACGCTGTTGCGCAGAATTTTTTGAATTGTCCGA
TCTTCTTCTTCACAAAAAGAACTGCACGAAGAATCAGTTGGTTTTGATAG
TAAACGAAAATCCAGCTTCACCCCCAGAAACTTTTTCCCCGTCACCTCCT
CCAGATAATCCTGATGAACAAATGAATGACACCGTAAATAAAACCGACCA
AGTAGACTGTTCTGATTTGAGCGAACACAACGGTTTGGATCGAGAAGAGT
CAATGGAAGTAGAGGCCCCAGTTGCCAATAAGTCAGGCAGCGGTACTTCT
TCCGGCTCCCACAGTTCAACAGCTCCATCCTCAAGTAGTTCAAGCTCTTC
TAGTTCAGGAGGCGGGGGGAGTAGCTCTACCGGCACTTCTGCCATCACAA
CCTCACTTCCTCAGCTTGGAGACTTGACAGGATCCGGTGGGGGATCTGGG
GGATCTGGCTCGATGTCTGCAGCTGGCCTTTTGGCTCCTGCCCCCGCACA
AGCGGGAGCTCCTCCCGCACCGGAGTACTATCCAGAAGAGGATGAGGAAC
TGGAATCTGCCGAAGACGACGAGCGCAGTTGCCGGGGGAGGGAATCTGAC
GAGGATACTGAGGATGCTTCTGAGACCGACCTCGCGAAACATGATGAGGA
AGACTACGTTGAAATGAAAGAGCAGATGTACCAAGACAAACTTGCTAGCC
TCAAGAGACAGTTGCAGCAACTGCAAGAAGGCACGCTCCAGGAGTACCAG
AAGAGAATGAAAAAACTCGACCAGCAGTACAAGGAACGAATTAGAAACGC
AGAGCTCTTTCTTCAGCTGGAGACTGAACAGGTTGAGCGCAATTATATTA
AGGAAAAAAAAGCCGCTGTGAAGGAGTTCGAAGACAAGAAAGTGGAACTT
AAAGAAAACCTCATCGCCGAACTGGAGGAGAAGAAGAAGATGATAGAGAA
CGAAAAACTCACAATGGAACTGACGGGTGATTCCATGGAGGTAAAACCGA
TTATGACCCGAAAGCTCCGCCGACGCCCAAACGATCCGGTACCGATCCCT
GATAAGCGGCGCAAGCCCGCACCGGCTCAGCTCAATTACCTGCTGACCGA
CGAACAAATAATGGAGGACCTGCGGACTCTTAATAAGCTGAAGAGTCCTA
AACGGCCAGCTTCCCCCAGTTCCCCCGAACACCTGCCCGCTACTCCCGCG
GAGAGCCCTGCTCAGCGCTTTGAGGCCCGAATCGAGGACGGAAAATTGTA
CTATGACAAACGCTGGTATCATAAGAGCCAGGCTATATACCTGGAGTCAA
AAGATAACCAAAAGTTGTCATGTGTAATCTCCTCAGTCGGGGCTAACGAA
ATATGGGTGCGGAAGACCTCTGATAGTACGAAGATGCGCATATATCTGGG
ACAATTGCAAAGAGGACTTTTTGTTATAAGACGGAGAAGCGCTGCT

SUDS3-SALL1-Active Cas9 amino acid sequence:

(SEQ ID NO: 171)
MSAAGLLAPAPAQAGAPPAPEYYPEEDEELESAEDDERSCRGRESDEDTE
DASETDLAKHDEEDYVEMKEQMYQDKLASLKRQLQQLQEGTLQEYQKRMK
KLDQQYKERIRNAELFLQLETEQVERNYIKEKKAAVKEFEDKKVELKENL
IAELEEKKKMIENEKLTMELTGDSMEVKPIMTRKLRRRPNDPVPIPDKRR
KPAPAQLNYLLTDEQIMEDLRTLNKLKSPKRPASPSSPEHLPATPAESPA
QRFEARIEDGKLYYDKRWYHKSQAIYLESKDNQKLSCVISSVGANEIWVR
KTSDSTKMRIYLGQLQRGLFVIRRRSAAGSGGGSGGSGSMSRRKQAKPQH
FQSDPEVASLPRRDGDTEKGQPSRPTKSKDAHVCGRCCAEFFELSDLLLH
KKNCTKNQLVLIVNENPASPPETFSPSPPPDNPDEQMNDTVNKTDQVDCS
DLSEHNGLDREESMEVEAPVANKSGSGTSSGSHSSTAPSSSSSSSSSSGG
GGSSSTGTSAITTSLPQLGDLTGSGGGSGGSGSMDYKDDDDKMAPKKKRK
VGIHGVPAADKKYSIGLDIGTNSVGWAVITDEYKVPSKKFKVLGNTDRHS
IKKNLIGALLFDSGETAEATRLKRTARRRYTRRKNRICYLQEIFSNEMAK
VDDSFFHRLEESFLVEEDKKHERHPIFGNIVDEVAYHEKYPTIYHLRKKL
VDSTDKADLRLIYLALAHMIKFRGHFLIEGDLNPDNSDVDKLFIQLVQTY
NQLFEENPINASGVDAKAILSARLSKSRRLENLIAQLPGEKKNGLFGNLI
ALSLGLTPNFKSNFDLAEDAKLQLSKDTYDDDLDNLLAQIGDQYADLFLA
AKNLSDAILLSDILRVNTEITKAPLSASMIKRYDEHHQDLTLLKALVRQQ
LPEKYKEIFFDQSKNGYAGYIDGGASQEEFYKFIKPILEKMDGTEELLVK
LNREDLLRKQRTFDNGSIPHQIHLGELHAILRRQEDFYPFLKDNREKIEK
ILTFRIPYYVGPLARGNSRFAWMTRKSEETITPWNFEEVVDKGASAQSFI
ERMTNFDKNLPNEKVLPKHSLLYEYFTVYNELTKVKYVTEGMRKPAFLSG
EQKKAIVDLLFKTNRKVTVKQLKEDYFKKIECFDSVEISGVEDRFNASLG
TYHDLLKIIKDKDFLDNEENEDILEDIVLTLTLFEDREMIEERLKTYAHL
FDDKVMKQLKRRRYTGWGRLSRKLINGIRDKQSGKTILDFLKSDGFANRN
FMQLIHDDSLTFKEDIQKAQVSGQGDSLHEHIANLAGSPAIKKGILQTVK
VVDELVKVMGRHKPENIVIEMARENQTTQKGQKNSRERMKRIEEGIKELG
SQILKEHPVENTQLQNEKLYLYYLQNGRDMYVDQELDINRLSDYDVDHIV
PQSFLKDDSIDNKVLTRSDKNRGKSDNVPSEEVVKKMKNYWRQLLNAKLI
TQRKFDNLTKAERGGLSELDKAGFIKRQLVETRQITKHVAQILDSRMNTK
YDENDKLIREVKVITLKSKLVSDFRKDFQFYKVREINNYHHAHDAYLNAV
VGTALIKKYPKLESEFVYGDYKVYDVRKMIAKSEQEIGKATAKYFFYSNI
MNFFKTEITLANGEIRKRPLIETNGETGEIVWDKGRDFATVRKVLSMPQV
NIVKKTEVQTGGFSKESILPKRNSDKLIARKKDWDPKKYGGFDSPTVAYS
VLVVAKVEKGKSKKLKSVKELLGITIMERSSFEKNPIDFLEAKGYKEVKK
DLIIKLPKYSLFELENGRKRMLASAGELQKGNELALPSKYVNFLYLASHY
EKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEV
LDATLIHQSITGLYETRIDLSQLGGDKRPAATKKAGQAKKKK

SEQ ID NO: 171 may, for example be coded by nucleic acid comprises, consisting essentially of or consisting of SEO ID NO: 172:

ATGTCTGCAGCTGGCCTTTTGGCTCCTGCCCCCGCACAAGCGGGAGCTCC
TCCCGCACCGGAGTACTATCCAGAAGAGGATGAGGAACTGGAATCTGCCG
AAGACGACGAGCGCAGTTGCCGGGGGAGGGAATCTGACGAGGATACTGAG
GATGCTTCTGAGACCGACCTCGCGAAACATGATGAGGAAGACTACGTTGA
AATGAAAGAGCAGATGTACCAAGACAAACTTGCTAGCCTCAAGAGACAGT
TGCAGCAACTGCAAGAAGGCACGCTCCAGGAGTACCAGAAGAGAATGAAA
AAACTCGACCAGCAGTACAAGGAACGAATTAGAAACGCAGAGCTCTTTCT
TCAGCTGGAGACTGAACAGGTTGAGCGCAATTATATTAAGGAAAAAAAAG
CCGCTGTGAAGGAGTTCGAAGACAAGAAAGTGGAACTTAAAGAAAACCTC
ATCGCCGAACTGGAGGAGAAGAAGAAGATGATAGAGAACGAAAAACTCAC
AATGGAACTGACGGGTGATTCCATGGAGGTAAAACCGATTATGACCCGAA
AGCTCCGCCGACGCCCAAACGATCCGGTACCGATCCCTGATAAGCGGCGC
AAGCCCGCACCGGCTCAGCTCAATTACCTGCTGACCGACGAACAAATAAT
GGAGGACCTGCGGACTCTTAATAAGCTGAAGAGTCCTAAACGGCCAGCTT
CCCCCAGTTCCCCCGAACACCTGCCCGCTACTCCCGCGGAGAGCCCTGCT
CAGCGCTTTGAGGCCCGAATCGAGGACGGAAAATTGTACTATGACAAACG
CTGGTATCATAAGAGCCAGGCTATATACCTGGAGTCAAAAGATAACCAAA
AGTTGTCATGTGTAATCTCCTCAGTCGGGGCTAACGAAATATGGGTGCGG
AAGACCTCTGATAGTACGAAGATGCGCATATATCTGGGACAATTGCAAAG
AGGACTTTTTGTTATAAGACGGAGAAGCGCTGCTGGATCCGGTGGGGGAT
CTGGGGGATCTGGCTCGATGAGTAGGAGAAAACAAGCAAAACCACAGCAC
TTTCAAAGTGATCCTGAGGTAGCAAGCCTTCCACGGCGGGACGGTGACAC
GGAGAAGGGTCAACCAAGTCGACCCACGAAAAGCAAAGATGCTCATGTAT
GTGGACGCTGTTGCGCAGAATTTTTTGAATTGTCCGATCTTCTTCTTCAC
AAAAAGAACTGCACGAAGAATCAGTTGGTTTTGATAGTAAACGAAAATCC
AGCTTCACCCCCAGAAACTTTTTCCCCGTCACCTCCTCCAGATAATCCTG
ATGAACAAATGAATGACACCGTAAATAAAACCGACCAAGTAGACTGTTCT
GATTTGAGCGAACACAACGGTTTGGATCGAGAAGAGTCAATGGAAGTAGA
GGCCCCAGTTGCCAATAAGTCAGGCAGCGGTACTTCTTCCGGCTCCCACA
GTTCAACAGCTCCATCCTCAAGTAGTTCAAGCTCTTCTAGTTCAGGAGGC
GGGGGGAGTAGCTCTACCGGCACTTCTGCCATCACAACCTCACTTCCTCA
GCTTGGAGACTTGACAGGATCCGGTGGGGGATCTGGGGGATCTGGCTCGA
TGGATTACAAAGACGATGACGATAAGATGGCCCCAAAGAAGAAGCGGAAG
GTCGGTATCCACGGAGTCCCAGCAGCCGACAAGAAGTACAGCATCGGCCT
GGACATCGGCACCAACTCTGTGGGCTGGGCCGTGATCACCGACGAGTACA
AGGTGCCCAGCAAGAAATTCAAGGTGCTGGGCAACACCGACCGGCACAGC
ATCAAGAAGAACCTGATCGGAGCCCTGCTGTTCGACAGCGGCGAAACAGC
CGAGGCCACCCGGCTGAAGAGAACCGCCAGAAGAAGATACACCAGACGGA
AGAACCGGATCTGCTATCTGCAAGAGATCTTCAGCAACGAGATGGCCAAG
GTGGACGACAGCTTCTTCCACAGACTGGAAGAGTCCTTCCTGGTGGAAGA
GGATAAGAAGCACGAGCGGCACCCCATCTTCGGCAACATCGTGGACGAGG
TGGCCTACCACGAGAAGTACCCCACCATCTACCACCTGAGAAAGAAACTG
GTGGACAGCACCGACAAGGCCGACCTGCGGCTGATCTATCTGGCCCTGGC
CCACATGATCAAGTTCCGGGGCCACTTCCTGATCGAGGGCGACCTGAACC
CCGACAACAGCGACGTGGACAAGCTGTTCATCCAGCTGGTGCAGACCTAC
AACCAGCTGTTCGAGGAAAACCCCATCAACGCCAGCGGCGTGGACGCCAA
GGCCATCCTGTCTGCCAGACTGAGCAAGAGCAGACGGCTGGAAAATCTGA
TCGCCCAGCTGCCCGGCGAGAAGAAGAATGGCCTGTTCGGCAACCTGATT
GCCCTGAGCCTGGGCCTGACCCCCAACTTCAAGAGCAACTTCGACCTGGC
CGAGGATGCCAAACTGCAGCTGAGCAAGGACACCTACGACGACGACCTGG
ACAACCTGCTGGCCCAGATCGGCGACCAGTACGCCGACCTGTTTCTGGCC
GCCAAGAACCTGTCCGACGCCATCCTGCTGAGCGACATCCTGAGAGTGAA
CACCGAGATCACCAAGGCCCCCCTGAGCGCCTCTATGATCAAGAGATACG
ACGAGCACCACCAGGACCTGACCCTGCTGAAAGCTCTCGTGCGGCAGCAG
CTGCCTGAGAAGTACAAAGAGATTTTCTTCGACCAGAGCAAGAACGGCTA
CGCCGGCTACATTGACGGCGGAGCCAGCCAGGAAGAGTTCTACAAGTTCA
TCAAGCCCATCCTGGAAAAGATGGACGGCACCGAGGAACTGCTCGTGAAG
CTGAACAGAGAGGACCTGCTGCGGAAGCAGCGGACCTTCGACAACGGCAG
CATCCCCCACCAGATCCACCTGGGAGAGCTGCACGCCATTCTGCGGCGGC
AGGAAGATTTTTACCCATTCCTGAAGGACAACCGGGAAAAGATCGAGAAG
ATCCTGACCTTCCGCATCCCCTACTACGTGGGCCCTCTGGCCAGGGGAAA
CAGCAGATTCGCCTGGATGACCAGAAAGAGCGAGGAAACCATCACCCCCT
GGAACTTCGAGGAAGTGGTGGACAAGGGCGCTTCCGCCCAGAGCTTCATC
GAGCGGATGACCAACTTCGATAAGAACCTGCCCAACGAGAAGGTGCTGCC
CAAGCACAGCCTGCTGTACGAGTACTTCACCGTGTATAACGAGCTGACCA
AAGTGAAATACGTGACCGAGGGAATGAGAAAGCCCGCCTTCCTGAGCGGC
GAGCAGAAAAAGGCCATCGTGGACCTGCTGTTCAAGACCAACCGGAAAGT
GACCGTGAAGCAGCTGAAAGAGGACTACTTCAAGAAAATCGAGTGCTTCG
ACTCCGTGGAAATCTCCGGCGTGGAAGATCGGTTCAACGCCTCCCTGGGC
ACATACCACGATCTGCTGAAAATTATCAAGGACAAGGACTTCCTGGACAA
TGAGGAAAACGAGGACATTCTGGAAGATATCGTGCTGACCCTGACACTGT
TTGAGGACAGAGAGATGATCGAGGAACGGCTGAAAACCTATGCCCACCTG
TTCGACGACAAAGTGATGAAGCAGCTGAAGCGGCGGAGATACACCGGCTG
GGGCAGGCTGAGCCGGAAGCTGATCAACGGCATCCGGGACAAGCAGTCCG
GCAAGACAATCCTGGATTTCCTGAAGTCCGACGGCTTCGCCAACAGAAAC
TTCATGCAGCTGATCCACGACGACAGCCTGACCTTTAAAGAGGACATCCA
GAAAGCCCAGGTGTCCGGCCAGGGCGATAGCCTGCACGAGCACATTGCCA
ATCTGGCCGGCAGCCCCGCCATTAAGAAGGGCATCCTGCAGACAGTGAAG
GTGGTGGACGAGCTCGTGAAAGTGATGGGCCGGCACAAGCCCGAGAACAT
CGTGATCGAAATGGCCAGAGAGAACCAGACCACCCAGAAGGGACAGAAGA
ACAGCCGCGAGAGAATGAAGCGGATCGAAGAGGGCATCAAAGAGCTGGGC
AGCCAGATCCTGAAAGAACACCCCGTGGAAAACACCCAGCTGCAGAACGA
GAAGCTGTACCTGTACTACCTGCAGAATGGGCGGGATATGTACGTGGACC
AGGAACTGGACATCAACCGGCTGTCCGACTACGATGTGGACCATATCGTG
CCTCAGAGCTTTCTGAAGGACGACTCCATCGACAACAAGGTGCTGACCAG
AAGCGACAAGAACCGGGGCAAGAGCGACAACGTGCCCTCCGAAGAGGTCG
TGAAGAAGATGAAGAACTACTGGCGGCAGCTGCTGAACGCCAAGCTGATT
ACCCAGAGAAAGTTCGACAATCTGACCAAGGCCGAGAGAGGCGGCCTGAG
CGAACTGGATAAGGCCGGCTTCATCAAGAGACAGCTGGTGGAAACCCGGC
AGATCACAAAGCACGTGGCACAGATCCTGGACTCCCGGATGAACACTAAG
TACGACGAGAATGACAAGCTGATCCGGGAAGTGAAAGTGATCACCCTGAA
GTCCAAGCTGGTGTCCGATTTCCGGAAGGATTTCCAGTTTTACAAAGTGC
GCGAGATCAACAACTACCACCACGCCCACGACGCCTACCTGAACGCCGTC
GTGGGAACCGCCCTGATCAAAAAGTACCCTAAGCTGGAAAGCGAGTTCGT
GTACGGCGACTACAAGGTGTACGACGTGCGGAAGATGATCGCCAAGAGCG
AGCAGGAAATCGGCAAGGCTACCGCCAAGTACTTCTTCTACAGCAACATC
ATGAACTTTTTCAAGACCGAGATTACCCTGGCCAACGGCGAGATCCGGAA
GCGGCCTCTGATCGAGACAAACGGCGAAACCGGGGAGATCGTGTGGGATA
AGGGCCGGGATTTTGCCACCGTGCGGAAAGTGCTGAGCATGCCCCAAGTG
AATATCGTGAAAAAGACCGAGGTGCAGACAGGCGGCTTCAGCAAAGAGTC
TATCCTGCCCAAGAGGAACAGCGATAAGCTGATCGCCAGAAAGAAGGACT
GGGACCCTAAGAAGTACGGCGGCTTCGACAGCCCCACCGTGGCCTATTCT
GTGCTGGTGGTGGCCAAAGTGGAAAAGGGCAAGTCCAAGAAACTGAAGAG
TGTGAAAGAGCTGCTGGGGATCACCATCATGGAAAGAAGCAGCTTCGAGA
AGAATCCCATCGACTTTCTGGAAGCCAAGGGCTACAAAGAAGTGAAAAAG
GACCTGATCATCAAGCTGCCTAAGTACTCCCTGTTCGAGCTGGAAAACGG
CCGGAAGAGAATGCTGGCCTCTGCCGGCGAACTGCAGAAGGGAAACGAAC
TGGCCCTGCCCTCCAAATATGTGAACTTCCTGTACCTGGCCAGCCACTAT
GAGAAGCTGAAGGGCTCCCCCGAGGATAATGAGCAGAAACAGCTGTTTGT
GGAACAGCACAAGCACTACCTGGACGAGATCATCGAGCAGATCAGCGAGT
TCTCCAAGAGAGTGATCCTGGCCGACGCTAATCTGGACAAAGTGCTGTCC
GCCTACAACAAGCACCGGGATAAGCCCATCAGAGAGCAGGCCGAGAATAT
CATCCACCTGTTTACCCTGACCAATCTGGGAGCCCCTGCCGCCTTCAAGT
ACTTTGACACCACCATCGACCGGAAGAGGTACACCAGCACCAAAGAGGTG
CTGGACGCCACCCTGATCCACCAGAGCATCACCGGCCTGTACGAGACACG
GATCGACCTGTCTCAGCTGGGAGGCGACAAGCGTCCTGCTGCTACTAAGA
AAGCTGGTCAAGCTAAGAAAAAGAAA

Within the scope of the present invention are proteins and polypeptide sequences that are fragments of SEQ ID NO: 41 and 171 and derivatives of those sequences that can be used to perform substantially similar functions. In some embodiments, the proteins or polypeptides are at least 80%, at least 85%, at least 90%, at least 95% similar to either SEQ ID NO: 41 and 171. Additionally, within the scope of the present invention are nucleic acid sequences comprises, consist essentially of, or consist of SEQ ID NO: 170 or 172 or complement thereof, or sequences that are at least 80%, at least 85%, at least 90%, at least 95% similar to or complementary to either SEQ ID NO: 170 and 172.

### Linkers

When a repressor domain is fused to a Cas protein the fusion may be by a direct bond (e.g., a covalent bond) between the N terminal amino acid of the repressor protein and the C terminal amino acid of the Cas protein or the C terminal amino acid of the repressor protein and the N terminal amino acid of the Cas protein.

However, instead of directly linking or forming a bond between two components, i.e., two or more repressor domains or a repressor domain and a Cas protein, one may use a linker. In some embodiments, the linker comprises, consists essentially of, or consists of an amino acid sequence that is, e.g., 1 to 100 amino acid long or 3 to 90 amino acids long or 10 to 50 amino acids long. In some embodiments, the linker comprises, consists essentially of, or consists of a sequence that is not an amino acid sequence.

When the linker is between a Cas protein and a repressor domain, the linker may be referred to as a Cas linker. In some embodiments, the Cas protein has a C terminal amino acid and the Cas fusion protein comprises a Cas linker, wherein the Cas linker is covalently bound to the C terminal amino acid of the Cas protein. In some embodiments, the Cas protein has an N terminal amino acid and the Cas fusion protein comprises a Cas linker, wherein the Cas linker is covalently bound to the N terminal amino acid of the Cas protein. In some embodiments, the Cas protein has a C terminal amino acid and an N terminal amino acid and the Cas fusion protein comprises two Cas linkers, wherein a first Cas linker is covalently bound to the C terminal amino acid of the Cas protein and a second Cas linker is covalently bound to the N terminal amino acid of the Cas protein. When there are a first Cas linker and a second Cas linker, the first Cas linker may be bound to a first repressor domain and the second Cas linker may be bound to a second repressor domain.

In some embodiments, there is one Cas linker and the Cas linker comprises, consists essentially of, or consists of a sequence that is at least 80%, at least 85%, at least 90%, or at least 95% similar to SEQ ID NO: 7: GSGGGSGGSGS. In some embodiments, the Cas linker comprises, consists essentially of, or consists of a sequence that is SEQ ID NO: 7. In some embodiments, there are two Cas linkers and each Cas linker comprises, consists essentially of, or consists of a sequence that is at least 80%, at least 85%, at least 90%, or at least 95% similar to SEQ ID NO: 7: GSGGGSGGSGS. In some embodiments, each of two Cas linkers comprises, consists essentially of, or consists of a sequence that is SEQ ID NO: 7.

In some embodiments, the Cas linker is covalently bound to a Cas protein and a repressor domain that comprises, consists essentially of or consists of a sequence that is at least 80%, at least 85%, at least 90%, at least 95% similar to SEQ ID NO: 1. In some embodiments, the Cas linker is covalently bound to a Cas protein and a repressor domain that comprises, consists essentially of or consists of a sequence that is SEQ ID NO: 1.

In some embodiments, the Cas linker is covalently bound to a Cas protein and a repressor domain that comprises, consists essentially of or consists of a sequence that is at least 80%, at least 85%, at least 90%, at least 95% similar to SEQ ID NO: 2. In some embodiments, the Cas linker is covalently bound to a Cas protein and a repressor domain that comprises, consists essentially of or consists of a sequence that is SEQ ID NO: 2.

When the Cas fusion protein comprises two or more repressor domains and two or more repressor domains are on the same side of the Cas protein, i.e., on the N side or the C side, each pair of repressor domains may be directly, e.g., covalently bound to each other, or they may be joined through a linker. A linker that joins two repressor domains may be referred to as a repressor linker.

The repressor linker may be the same as or different from the Cas linker. In some embodiments, the repressor linker comprises, consists essentially of, or consists of a sequence that is at least 80%, at least 85%, at least 90%, or at least 95% similar to SEQ ID NO: 7: GSGGGSGGSGS. In some embodiments, the repressor linker comprises, consists essentially of, or consists of a sequence that is SEQ ID NO: 7.

By way of a non-limiting example, in a Cas fusion protein of the present invention, the Cas protein may be a dCas9 protein or dCas12 such as dCas12a protein, wherein the Cas protein has a C terminal amino acid and the Cas fusion protein further comprises a Cas linker and a repressor linker, wherein the Cas linker is covalently bound to the C terminal amino acid of the Cas protein and to the N terminal amino acid of the SALL1 repressor domain and wherein the repressor linker is between the SALL1 repressor domain and the SUDS3 repressor domain.

By way of another non-limiting example, in a Cas fusion protein of the present invention, the Cas protein may be a dCas9 protein or dCas12 such as dCas12a protein, wherein the Cas protein has a C terminal amino acid and the Cas fusion protein further comprises a Cas linker and a repressor linker, wherein the Cas linker is covalently bound to the C terminal amino acid of the Cas protein and to the SUDS3 repressor domain and wherein the repressor linker is bound to both the SUDS3 repressor domain and the SALL1 repressor domain.

By way of another non-limiting example, in a Cas fusion protein of the present invention, the Cas protein may be a dCas9 protein or dCas12 such as dCas12a protein, wherein the Cas protein has a N terminal amino acid and the Cas fusion protein further comprises a Cas linker and a repressor linker, wherein the Cas linker is covalently bound to the N terminal amino acid of the Cas protein and to the SUDS3 repressor domain and wherein the repressor linker is bound to both the SUDS3 repressor domain and the SALL1 repressor domain.

By way of another non-limiting example, in a Cas fusion protein of the present invention, the Cas protein may be a dCas9 protein or dCas12 such as dCas12a protein, wherein the Cas protein has a N terminal amino acid and the Cas fusion protein further comprises a Cas linker and a repressor linker, wherein the Cas linker is covalently bound to the N terminal amino acid of the Cas protein and to the SALL1 repressor domain and wherein the repressor linker is bound to both the SALL1 repressor domain and the SUDS3repressor domain.

By way of another non-limiting example, in a Cas fusion protein of the present invention, the Cas protein may be a dCas9 protein or dCas12 such as dCas12a protein, wherein the Cas protein has a N terminal amino acid and a C terminal amino acid and the Cas fusion protein further comprises a first Cas linker and a second Cas linker, wherein the first Cas linker is covalently bound to the N terminal amino acid of the Cas protein and to the SUDS3 repressor domain and wherein the second Cas linker is bound to the C terminus of Cas protein and to the SALL1 repressor domain.

By way of another non-limiting example, in a Cas fusion protein of the present invention, the Cas protein may be a dCas9 protein or dCas12 such as dCas12a protein, wherein the Cas protein has a N terminal amino acid and a C terminal amino acid and the Cas fusion protein further comprises a first Cas linker and a second Cas linker, wherein the first Cas linker is covalently bound to the C terminal amino acid of the Cas protein and to the SUDS3 repressor domain and wherein the second Cas linker is bound to the N terminal amino acid of Cas protein and to the SALL1 repressor domain.

By way of another example, in some embodiments, the Cas fusion protein comprises a sequence that is at least 80%, at least 85%, at least 90%, or at least 95% similar to SEQ ID NO: 10:

GSGGGSGGSGSMSRRKQAKPQHFQSDPEVASLPRRDGDTEKGQPSRPTKS
KDAHVCGRCCAEFFELSDLLLHKKNCTKNQLVLIVNENPASPPETFSPSP
PPDNPDEQMNDTVNKTDQVDCSDLSEHNGLDREESMEVEAPVANKSGSGT
SSGSHSSTAPSSSSSSSSSSGGGGSSSTGTSAITTSLPQLGDLTGSGGGS
GGSGSMSAAGLLAPAPAQAGAPPAPEYYPEEDEELESAEDDERSCRGRES
DEDTEDASETDLAKHDEEDYVEMKEQMYQDKLASLKRQLQQLQEGTLQEY
QKRMKKLDQQYKERIRNAELFLQLETEQVERNYIKEKKAAVKEFEDKKVE
LKENLIAELEEKKKMIENEKLTMELTGDSMEVKPIMTRKLRRRPNDPVPI
PDKRRKPAPAQLNYLLTDEQIMEDLRTLNKLKSPKRPASPSSPEHLPATP
AESPAQRFEARIEDGKLYYDKRWYHKSQAIYLESKDNQKLSCVISSVGAN
EIWVRKTSDSTKMRIYLGQLQRGLFVIRRRSAA.

In some embodiments, the Cas fusion protein comprises a sequence that the same as SEQ ID NO: 10.

In some embodiments, the Cas fusion protein is:
[dCas9]-[Cas linker]-[SALL1 repressor domain]-[repressor linker]-[SUDS3 repressor domain].

In some embodiments, the Cas fusion protein is:
[dCas9]-[Cas linker]-[SUDS3 repressor domain]-[repressor linker]-[SALL1 repressor domain].

In some embodiments, the Cas fusion protein is: [dMAD7]-[Cas linker]-[SALL1 repressor domain]-[repressor linker]-[SUDS3 repressor domain].

In some embodiments, the Cas fusion protein is:
[dMAD7]-[Cas linker]-[SUDS3 repressor domain]-[repressor linker]-[SALL1 repressor domain].

In some embodiments, the Cas fusion protein is:
[SALL1 repressor domain]-[repressor linker]-[SUDS3 repressor domain]-[Cas linker]-[dCas9].

In some embodiments, the Cas fusion protein is:
[SUDS3 repressor domain]-[repressor linker]-[SALL1 repressor domain]-[Cas linker]-[dCas9].

In some embodiments, the Cas fusion protein is:
[SALL1 repressor domain]-[repressor linker]-[SUDS3 repressor domain]-[Cas linker]-[dMAD7].

In some embodiments, the Cas fusion protein is:
[SUDS3 repressor domain]-[repressor linker]-[SALL1 repressor domain]-[Cas linker]-[dMAD7].
   gRNA

The Cas-fusion proteins of the present invention may be used in conjunction with gRNAs. In some embodiments, the gRNA contains 30 to 180 nucleotide or 45 to 135 nucleotides or 60 to 120 nucleotides. A gRNA may be chemically synthesized or enzymatically synthesized. When enzymatically synthesized, the synthesis may occur in vitro, in vivo, or ex vivo.

The nucleotides of the gRNA may be exclusively modified ribonucleotides, exclusively unmodified ribonucleotides, or a combination or modified and unmodified ribonucleotides. In some embodiments, the gRNA contains one or more modification such as 2′ modifications, e.g., 2-O-alkyl such as 2′-O-methyl or 2′-O-ethyl, or 2′-halogenmodifications such as 2′ Fluoro. In some embodiments, the gRNA contains one or more modified internucleotide linkages such a phosphorothioate linkages.

In some embodiments, the gRNA has the following modifications:
2′-O-methyl modifications on the first and second 5′ most nucleotides,
2′-O-methyl modifications on the penultimate 3′ nucleotide (second 3′ most nucleotide) and the antepenultimate 3′ nucleotide (third 3′ most nucleotide)
all other nucleotides are unmodified at their 2′ positions,
phosphorothioate linkages between the first and second 5′ most nucleotides, the second and third 5′ most nucleotides, the antepenultimate 3′ nucleotide and the penultimate 3′ nucleotide, and the penultimate 3′ nucleotide and the 3′ most nucleotide, and
all other internucleotide linkages are phosphodiester linkages.

In some embodiments, the gRNA has the following modifications:
2′-O-methyl modifications on the first and second 5′ most nucleotides,
all other nucleotides are unmodified at their 2′ positions,
phosphorothioate linkages between the first and second 5′ most nucleotides, the second and third 5′ most nucleotides, and
all other internucleotide linkages are phosphodiester linkages.

In some embodiments, the gRNA comprises, consists essentially of or consists of a crRNA. In some embodiments, the gRNA comprises, consists essentially of or consists of a crRNA sequence and a tracrRNA sequence. When the gRNA comprises, consists essentially of or consists of a crRNA sequence and a tracrRNA sequence, the crRNA and the tracrRNA may be part of a sgRNA or they each may be on a separate strand of nucleotides and form a crRNA molecule and a tracrRNA molecule, each of which is a polynucleotide. When they are part of two separate nucleotides, one of the tracrRNA molecule and the crRNA molecule may be referred to as a first RNA molecule and the other of the other tracrRNA molecule and the crRNA molecule may be referred to as a second RNA molecule. When there is a separate tracrRNA molecule and crRNA molecule, the total number of nucleotides in those two molecules combined may, for example, be the same as in the sgRNA described in various embodiments of the present invention. Further, any chemical modifications to nucleotides of sgRNAs may be present in either or both of the tracrRNA molecule and crRNA molecule, and any internucleotide modifications of sgRNAs may be present in either or both of the tracrRNA molecule and crRNA molecule. Additionally, any moieties described as being present on the 5′ end or 3′ end of a gRNA may in the case of a sgRNA be present on the 5′ end or 3′ end of the sgRNA, and in the case of separate tracrRNA molecules and crRNA molecules, each of which has a 5′ end or 3′ end, be present on the 5′ end or 3′ end of the tracrRNA molecule or crRNA molecule.

The crRNA comprises, consists essentially of or consists of a Cas association region and a spacer region (also referred to as a targeting region). The targeting region is sufficiently complementary to and capable of hybridizing to a pre-selected target site of interest. In various embodiments, the target specifying component of the guide sequence can comprise from about 10 nucleotides to more than about 25 nucleotides, for example up to 36 nucleotides. In some embodiments, the region of base pairing between the guide sequence and the corresponding target site sequence is about 10, 11, 12, 13, 14, 15, 16, 17, 18, 19, 20, 22, 23, 24, 25, or more than 25 nucleotides in length. In some embodiments, the targeting region is 12 to 30 nucleotides long, or 14 to 25 nucleotides long or about 17 to 20 nucleotides long or about 14 nucleotides long or about 20 nucleotides long. The targeting region may be at least 80%, at least 85%, at least 90%, at least 95%, at least 98% or 100% complementary to a region of the target dsDNA over at least 14 contiguous nucleotides, at least 15 contiguous nucleotides, at least 16 contiguous nucleotides, at least 17 contiguous nucleotides, at least 18 contiguous nucleotides, at least 19 contiguous nucleotides, at least 20 contiguous nucleotides, or 14 to 20 contiguous nucleotides.

When the targeting region is about 20 nucleotides long and used with an active Cas protein that is capable of cleaving both DNA strands, a double-strand break will be generated on the targeted DNA that can lead to insertions and/or deletions (indel) in the genome. If one wishes to cause repression without creating indels, one may either use an inactive Cas protein, such as a deactivated Cas9 protein. If using an active Cas protein that is generally capable of cleaving both DNA strands or a Cas nickase variant that is generally capable of cleaving one strand of the targeted DNA, one may use a gRNA that has a shorter targeting region, such as about 14 nucleotides long for gene repression. Guides with a 20 nt targeting region can lead the active Cas9-repressor to another genomic site for DNA cleaving and subsequent editing.

The Cas association region, which may for example, be about 18-36 nucleotides long is the portion of the crRNA that allows the crRNA (and thus the gRNA to retain association with the Cas protein). In some embodiments, association with the Cas protein is possible in the absence of a tracrRNA. In other embodiments, association requires the presence of a tracrRNA.

When a crRNA requires a tracrRNA to be present for association with the Cas protein, the Cas association region hybridizes with an anti-repeat region within the tracrRNA. The tracrRNA may also contain a distal region that is 3′ of the anti-repeat region and is not complementary to any region of the crRNA.

When there is hybridization between the Cas association region, which is also referred to as a repeat region and the anti-repeat region, the repeat: anti-repeat region of the gRNA scaffold can be split into 3 parts: the lower stem, bulge, and upper stem. The lower stem is 6 base pairs in length and forms through both Watson-Crick and no Watson-Crick base pairing; this is followed by a bulge structure of 6 nucleotides. Finally there is an upper stem that consists of a 4 base pair structure.

When the gRNA is a sgRNA, in some embodiments, the single strand may contain regions that are complementary and that when the complementary regions hybridize allow association with a Cas protein such as Type II Cas enzymes, including but not limited to Cas9 in active or deactivated form, and Type V Cas enzymes such as Cas12c, Cas12d, Cas12e, and Cas12f in active or deactivated form. In other embodiments, when the gRNA is a sgRNA, there are no regions that are complementary, but the sgRNA is capable of association with a Cas enzyme, such as certain Type V Cas enzymes such as Cas12a, MAD7 (an engineered variant of ErCas12a), Cas12h, Cas12i, and Cas12j (Casϕ) in active or deactivated form.

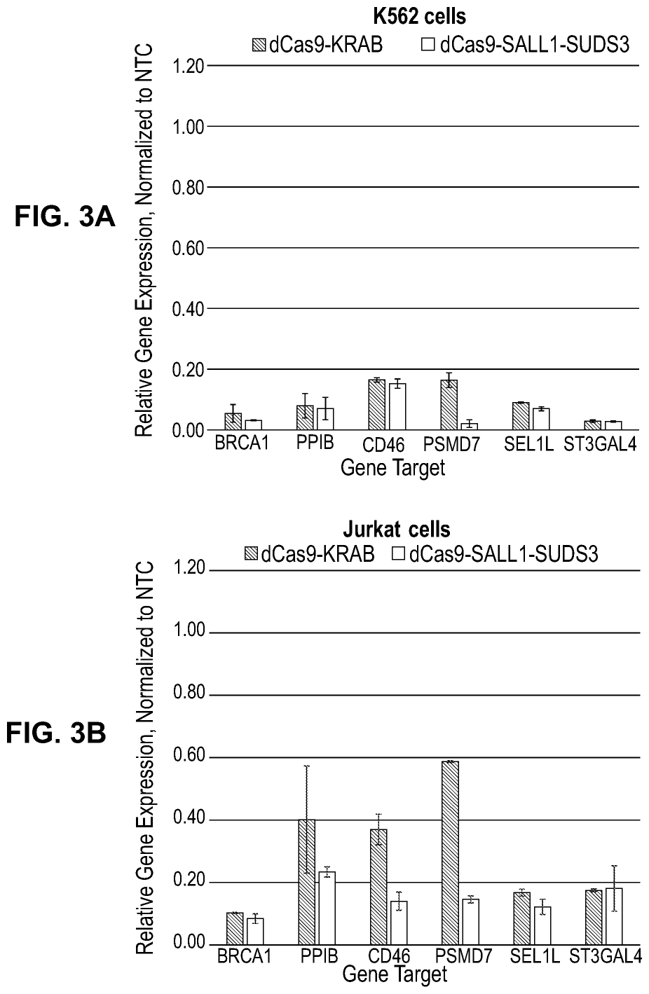

A non-limiting example of an sgRNA is shown in FIG. 2. The sgRNA of FIG. 2 has the following sequence: (SEQ ID NO: 11):

5′mN*mN*NNNNNNNNNNNNNNNNNNGUUUUAGAGCUAGAAAUAGCAAGU
UAAAAUAAGGCUAGUCCGUUAUCAACUUGAAAAAGUGGCACCGAGUCGGU
GCUmU*mU*U3′

m signifies a 2′O-methyl group;
* signifies a phosphorothioate linkage; and
N signifies any of A, C, G, or U.

As shown in FIG. 2, the crRNA region and the tracrRNA regions are joined by a tetra loop and the tracrRNA region has three stem loop regions. This example of an sgRNA has 100 nucleotides. In some embodiments, the sgRNA is 60 to 120 nucleotides long or 90 to 110 nucleotides long.

The N region as shown is 20 nucleotides long. In some embodiments, the N region is 10 to 36 nucleotides long or 14 to 26 nucleotides long or 18 to 22 nucleotides long.

Various tracrRNA sequences are known in the art and examples include SEQ ID Nos: 27-34, as well as active portions thereof.

(SEQ ID NO: 28)
GGAACCAUUCAAAACAGCAUAGCAAGUUAAAAUAAGGCUAGUCCGUUAUC
AACUUGAAAAAGUGGCACCGAGUCGGUGC;
(SEQ ID NO: 29)
UAGCAAGUUAAAAUAAGGCUAGUCCGUUAUCAACUUGAAAAAGUGGCACC
GAGUCGGUGC;
(SEQ ID NO: 30)
AGCAUAGCAAGUUAAAAUAAGGCUAGUCCGUUAUCAACUUGAAAAAGUGG
CACCGAGUCGGUGC;
(SEQ ID NO: 31)
CAAAACAGCAUAGCAAGUUAAAAUAAGGCUAGUCCGUUAUCAACUUGAAA
AAGUGGCACCGAGUCGGUGC;
(SEQ ID NO: 32)
UAGCAAGUUAAAAUAAGGCUAGUCCGUUAUCAACUUGAAAAAGUG;
(SEQ ID NO: 33)
UAGCAAGUUAAAAUAAGGCUAGUCCGUUAUCA;
and
(SEQ ID NO: 34)
UAGCAAGUUAAAAUAAGGCUAGUCCG.

As used herein, an active portion of a tracrRNA retains the ability to form a complex with a Cas protein, such as Cas9 or dCas9 or nCas9.

By way of a non-limiting example, the gRNA can be a hybrid RNA molecule where the above-described crRNA comprises a programmable gRNA fused to a tracrRNA to mimic the natural crRNA:tracrRNA duplex. An example of this type of hybrid is crRNA:tracrRNA, gRNA sequence: 5′-(20 nt guide)-

(SEQ ID NO: 27)
GUUUAAGAGCUAUGCUGGAAACAGCAUAGCAAGUUUAAAUAAGGCUAGUC
CGUUAUCAACUUGAAAAAGUGGCACCGAGUCGGUGCUUUUUUU-3′.

Methods for generating crRNA-tracrRNA hybrid RNAs (also known as sgRNAs) are known in the art. In one embodiment in which the crRNA and tracrRNA are provided as a sgRNA, the two components are linked together via a tetra stem loop. In some embodiments, the repeat anti-repeat region is extended. There may, for example, be an extension of 2, 3, 4, 5, 6, 7 bases or more than 7 bases at either side of the repeat: anti-repeat region. In another embodiment, the repeat: anti-repeat region has an extension of 7 nucleotides at either side of the stem. The extension of 7 bases at either side results in a region that is 14 base pairs longer. In other embodiments, the extension may be more than 7 bases. See e.g., WO2014099750, US 20140179006, and US 20140273226 for additional disclosure of tracrRNAs. The contents of these documents are incorporated herein by reference in their entireties.

In some embodiments the tracrRNA is from or derived from S. pyogenes.

In some embodiments, the target site resides on DNA. Within the DNA, the target nucleic acid strand can be either of the two strands and e.g., be in genomic DNA within a host cell. Examples of such genomic dsDNA include, but are not necessarily limited to, a host cell chromosome, mitochondrial DNA and a stably maintained plasmid. However, it is to be understood that the present method can be practiced on other dsDNA present in a host cell, such as non-stable plasmid DNA, viral DNA, and phagemid DNA, as long as there is Cas-targeted site.

In some embodiments, rather than using fusion proteins in combination with gRNA, one uses the fusion proteins of the present invention in combination with scoutRNA and the applicable crRNA. For example, the fusion proteins of the present invention may be used in a system or as part of a complex that has: (a) a crRNA, wherein the crRNA is 30 to 60 nucleotides long and the crRNA comprises a Cas association region and a targeting region, wherein the Cas association region is 15 to nucleotides long and the targeting region is 15 to 30 nucleotides long; (b) a scoutRNA, wherein the scoutRNA is 20 to 100 nucleotides long and wherein the scoutRNA comprises an anti-repeat region, wherein the anti-repeat region is 3 to 10 nucleotides long, and the anti-repeat region is complementary to at least 3 consecutive nucleotides within the Cas association region, and the anti-repeat region is capable of hybridizing with said at least 3 consecutive nucleotides within the Cas association region to form a hybridization region, wherein when the crRNA and scoutRNA form the hybridization region, and the crRNA and the scoutRNA are capable of retaining association with an RNA binding domain of a Type V Cas protein.

### RNA-Repressor Domain Complexes

In some embodiments, the present invention is directed to the use of an RNA-repressor domain complex. An RNA-repressor domain complex comprises, consists essentially of, or consists of: a gRNA such as a gRNA described above or a scoutRNA and/or a crRNA capable of associating with a scoutRNA as described above, a ligand binding moiety, a ligand, and one or more repressor domains. The RNA-repressor domain complexes may be used in conjunction with the Cas-fusion proteins of the present invention or with other Cas proteins that are not fusion proteins.

The gRNA or scoutRNA or crRNA capable of associating with a scoutRNA may be fused directly to a ligand binding moiety or associated with a ligand binding moiety through a ligand binding moiety linker. The ligand binding moiety is capable of reversibly associating with a ligand. The ligand is directly or through a ligand linker fused to a repressor domain. The repressor domain may be any effector. Each of the ligand binding moiety linker and the ligand linker if either or both are present may comprise, consist essentially of or consist of nucleotide(s), amino acids and other organic and inorganic moieties and combinations thereof.

A non-exhaustive list of examples of ligand binding moiety-ligand pairs that may be used in various embodiments of the present invention is provided in Table 1. Both unmodified and chemically modified versions or the ligand binding moieties and ligands are within the scope of the present invention.

TABLE 1

| Ligand Binding Moieties | Ligands |
|---|---|
| Telomerase Ku binding motif | Ku |
| Telomerase Sm7 binding motif | Sm7 |
| MS2 phage operator stem-loop | MS2 Coat Protein (MCP) |
| PP7 phage operator stem-loop | PP7 coat protein (PCP) |
| Qbeta phage operator stem-loop | Qbeta coat protein [Q65H] |
| SfMu phage Com stem-loop | Com RNA binding protein |
| Non-natural RNA aptamer | Corresponding aptamer ligand |
| Biotin | Streptavidin |
| Oligosaccharide | Lectin |
| Benzylguanine or benzylcytosine | SNAP/CLIP tag |
| 6x-His binding motif | 6x-His tag |
| PDGFbeta chain binding motif | PDGF B-chain |
| GST binding motif | GST protein |
| Tat binding motif | BIV Tat protein |
| Tat binding motif | HIV Tat protein |
| Pumilio binding motif | PUM-HD domain |
| BoxB binding motif | Lambda N22plus |
| Csy4 binding motif | Csy4[H29A] |

### 1. Telomerase Ku Binding Motif/Ku Heterodimer

a. Ku Binding Hairpin

(SEQ ID NO: 12)
5′-UUCUUGUCGUACUUAUAGAUCGCUACGUUAUUUCAAUUUUGAAAAUC
UGAGUCCUGGGAGUGCGGA-3′

b. Heterodimer

(SEQ ID No: 13)
MSGWESYYKTEGDEEAEEEQEENLEASGDYKYSGRDSLIFLVDASKAMFE
SQSEDELTPFDMSIQCIQSVYISKIISSDRDLLAVVFYGTEKDKNSVNFK
NIYVLQELDNPGAKRILELDQFKGQQGQKRFQDMMGHGSDYSLSEVLWVC
ANLFSDVQFKMSHKRIMLFTNEDNPHGNDSAKASRARTKAGDLRDTGIFL
DLMHLKKPGGFDISLFYRDIISIAEDEDLRVHFEESSKLEDLLRKVRAKE
TRKRALSRLKLKLNKDIVISVGIYNLVQKALKPPPIKLYRETNEPVKTKT
RTENTSTGGLLLPSDTKRSQIYGSRQIILEKEETEELKRFDDPGLMLMGF
KPLVLLKKHHYLRPSLFVYPEESLVIGSSTLFSALLIKCLEKEVAALCRY
TPRRNIPPYFVALVPQEEELDDQKIQVTPPGFQLVFLPFADDKRKMPFTE
KIMATPEQVGKMKAIVEKLRFTYRSDSFENPVLQQHFRNLEALALDLMEP
EQAVDLTLPKVEAMNKRLGSLVDEFKELVYPPDYNPEGKVTKRKHDNEGS
GSKRPKVEYSEEELKTHISKGTLGKFTVPMLKEACRAYGLKSGLKKQELL
EALTKHFQD
(SEQ ID No: 14)
MVRSGNKAAVVLCMDVGFTMSNSIPGIESPFEQAKKVITMFVQRQVFAEN
KDEIALVLFGTDGTDNPLSGGDQYQNITVHRHLMLPDFDLLEDIESKIQP
GSQQADFLDALIVSMDVIQHETIGKKFEKRHIEIFTDLSSRFSKSQLDII
IHSLKKCDISERHSIHWPCRLTIGSNLSIRIAAYKSILQERVKKTWTVVD
AKTLKKEDIQKETVYCLNDDDETEVLKEDIIQGFRYGSDIVPFSKVDEEQ
MKYKSEGKCFSVLGFCKSSQVQRRFFMGNQVLKVFAARDDEAAAVALSSL
IHALDDLDMVAIVRYAYDKRANPQVGVAFPHIKHNYECLVYVQLPFMEDL
RQYMFSSLKNSKKYAPTEAQLNAVDALIDSMSLAKKDEKTDTLEDLFPTT
KIPNPRFQRLFQCLLHRALHPREPLPPIQQHIWNMLNPPAEVTTKSQIPL
SKIKTLFPLIEAKKKDQVTAQEIFQDNHEDGPTAK

### 2. Telomerase Sm7 Binding Motif/Sm7 Homoheptamer

c. Sm Consensus Site (Single Stranded)

| (SEQ ID NO: 15) | |
|---|---|
| | 5′-AAUUUUUGGA-3′ |

d. Monomeric Sm-Like Protein (Archaea)

(SEQ ID No: 16)
GSVIDVSSQRVNVQRPLDALGNSLNSPVIIKLKGDREFRGVLKS
FDLHMNLVLNDAEELEDGEVTRRLGTVLIRGDNIVYISP

### 3. MS2 Phage Operator Stem Loop/MS2 Coat Protein

a. MS2 Phage Operator Stem Loop

(SEQ ID No: 17)
5′-GCACAUGAGGAUCACCCAUGUGC-3′

b. MS2 Coat Protein

(SEQ ID No: 18)
MASNFTQFVLVDNGGTGDVTVAPSNFANGIAEWISSNSRSQ
AYKVTCSVRQSSAQNRKYTIKVEVPKGAWRSYLNMELTIPI
FATNSDCELIVKAMQGLLKDGNPIPSAIAANSGIY

### 4. PP7 Phage Operator Stem Loop/PP7 Coat Protein

a. PP7 Phage Operator Stem Loop

(SEQ ID No: 19)
5′-AUAAGGAGUUUAUAUGGAAACCCUUA-3′

b. PP7 Coat Protein (PCP)

(SEQ ID No: 20)
MSKTIVLSVGEATRTLTEIQSTADRQIFEEKVGPLVGRLRLTASL
RQNGAKTAYRVNLKLDQADVVDCSTSVCGELPKVRYTQVWSHDVT
IVANSTEASRKSLYDLTKSLVATSQVEDLVVNLVPLGR.

### 5. SfMu Com Stem Loop/SfMu Com Binding Protein

a. SfMu Com Stem Loop

(SEQ ID No: 21)
5′-CUGAAUGCCUGCGAGCAUC-3′

b. SfMu Com Binding Protein

(SEQ ID No: 22)
MKSIRCKNCNKLLFKADSFDHIEIRCPRCKRHIIMLNACEHPTEK
HCGKREKITHSDETVRY

### 6. BoxB Aptamer/Lambda N22plus

e. BoxB Aptamer

(SEQ ID No: 23)
5′-GCCCUGAAGAAGGGC-3′

f. Lambda N22plus Protein

(SEQ ID No: 24)
MNARTRRRERRAEKQAQWKAAN

### 7. Csy4 Binding Stem Loop/Csy4[H29A]

a. Csy4 Binding Motif

(SEQ ID No: 25)
5′-CUGCCGUAUAGGCAGC-3′

b. Csy4[H29A]

(SEQ ID No: 26)
MDHYLDIRLRPDPEFPPAQLMSVLFGKLAQALVAQGGDRIGVSFPDLD
ESRSRLGERLRIHASADDLRALLARPWLEGLRDHLQFGEPAVVPHPTP
YRQVSRVQAKSNPERLRRRLMRRHDLSEEEARKRIPDTVARALDLPFV
TLRSQSTGQHFRLFIRHGPLQVTAEEGGFTCYGLSKGGFVPWF

### 8. Qbeta Binding Stem Loop [Q65H]

a. Qbeta Phage Operator Stem Loop

(SEQ ID No: 180)
5′-ATGCTGTCTAAGACAGCAT-3′

b. Qbeta Coat Protein [Q65H]

(SEQ ID No: 181)
MAKLETVTLGNIGKDGKQTLVLNPRGVNPTNGVASLSQAGAVPALEKRVT
VSVSQPSRNRKNYKVHVKIQNPTACTANGSCDPSVTRQAYADVTFSFTQY
STDEERAFVRTELAALLASPLLIDAIDQLNPAY

In each of the aforementioned sequences, one may, for example, use the identical sequence or sequences that have one or more insertions, deletions or substitutions in one or both sequences of a binding pair. By way of a non-limiting example, for either or both members of a binding pair one may use a sequence that is at least 80%, at least 85%, at least 90%, at least 95%, or at least 98% the same as an aforementioned sequence.

In some embodiments, a complex is formed that comprises, consists essentially of, or consists of a Cas-fusion protein of the present invention and RNA-repressor domain complex of the present invention. Thus, if the Cas-fusion protein comprises a Cas protein fused to the repressor domain SUDS3, the ligand may be fused to SALL1 or to any other repressor domain that is now known or that comes to be known. Similarly, if the Cas-fusion protein comprises a Cas protein fused to the repressor domain SALL1, the ligand may be fused to SUDS3 or to any other repressor domain that is now known or that comes to be known. Further, in some embodiments, the Cas-fusion protein comprises, consists essentially of, or consists or a Cas protein, a SALL1 repressor domain and a SUDS3 repressor domain, and the RNA-repressor domain complex comprises a gRNA, a ligand binding moiety, a ligand and one or more repressor domains other that SALL1 or SUDS3. By way of non-limiting examples, the one or more repressor domains may be selected from the group consisting of NIPP1, KRAB and DNMT3A.

Alternatively, one can use the RNA-repressor domain complexes with Cas enzymes that are not part of Cas-fusion protein complexes. For example, the RNA-repressor domain complex may comprise a gRNA, a ligand-binding moiety and one or both of the SUDS3 repressor domain and the SALL1 repressor domain as defined above. A repressor linker as defined above may be present between the SUDS3 repressor domain and the SALL1 repressor domain, and the ligand may be attached directly or through a ligand linker to either one of the SALL1 repressor domain and the SUDS3 repressor domain.

Nucleic Acids that Encode Cas Fusion Proteins

In some embodiments, the present invention provides a nucleic acid that encodes for a fusion protein of the present invention. The nucleic acid may be single stranded, double stranded or have at least one region that is single stranded and at least one region that is double stranded. Further, the nucleic acid may comprise, consist essentially of, or consist of RNA or DNA.

In some embodiments, the nucleic acid that encodes the fusion protein only contains nucleotides for the fusion protein and any linkers that are present. In other embodiments, the nucleic acid that encodes the fusion protein is part of a larger nucleic acid or a vector.

In some embodiments, the present invention is directed to a vector that comprises a nucleic acid that encodes a fusion protein of the present invention. In some embodiments, the vector is a plasmid or a viral vector. When the vector is a viral vector, in some embodiments, the viral vector is a lentiviral vector. In some embodiments, rather than a vector that comprises a polynucleotide sequence that encodes a Cas fusion protein, the present invention is directed to an mRNA that encodes a Cas fusion protein of the present invention.

In some embodiments, the nucleic acid comprises a sequence that encodes a Cas protein and at least one repressor domain such as SUDS3 or SALL1. In some embodiments, the nucleic acid comprises a sequence that encodes a Cas protein and at least two repressor domains, such as SUDS3 and SALL1. In some embodiments, the nucleic acid comprises a sequence that encodes a Cas protein and at least three repressor domains such as SUDS3, SALL1, and one or more of NIPP1, KRAB, and DNMT3A.

In some embodiments, the nucleic acid sequence comprises, consists essentially of, or consists of a sequence that is at least 80%, at least 85%, at least 90%, or at least 95% the same as or complementary to SEQ ID NO: 4, which encodes the SALL1 repressor domain:

ATG AGT AGG AGA AAA CAA GCA AAA CCA CAG CAC TTT
CAA AGT GAT CCT GAG GTA GCA AGC CTT CCA CGG GAC
GGT GAC ACG GAG AAG GGT CAA CCA AGT CGA CCC ACG
AAA AGC AAA GAT GCT CAT GTA TGT GGA CGC TGT TGC
GCA GAA TTT TTT GAA TTG TCC GAT CTT CTT CTT CAC
AAA AAG AAC TGC ACG AAG AAT CAG TTG GTT TTG ATA
GTA AAC GAA AAT CCA GCT TCA CCC CCA GAA ACT TTT
TCC CCG TCA CCT CCT CCA GAT AAT CCT GAT GAA CAA
ATG AAT GAC ACC GTA AAT AAA ACC GAC CAA GTA GAC
TGT TCT GAT TTG AGC GAA CAC AAC GGT TTG GAT CGA
GAA GAG TCA ATG GAA GTA GAG GCC CCA GTT GCC AAT
AAG TCA GGC AGC GGT ACT TCT TCC GGC TCC CAC AGT
TCA ACA GCT CCA TCC TCA AGT AGT TCA AGC TCT TCT
AGT TCA GGA GGC GGG GGG AGT AGC TCT ACC GGC ACT
TCT GCC ATC ACA ACC TCA CTT CCT CAG CTT GGA GAC
TTG ACA.

In some embodiments, the nucleic acid sequence comprises, consists essentially of, or consists of a sequence that is the same as SEQ ID NO: 4.

In some embodiments, the nucleic acid sequence comprises, consists essentially of, or consists of a sequence is at least 80%, at least 85%, at least 90%, or at least 95% the same as or complementary to SEQ ID NO: 5, which encode the SUDS3 repressor domain:

ATG TCT GCA GCT GGC CTT TTG GCT CCT GCC CCC GCA
CAA GCG GGA GCT CCT CCC GCA CCG GAG TAC TAT CCA
GAA GAG GAT GAG GAA CTG GAA TCT GCC GAA GAC GAC
GAG CGC AGT TGC CGG GGG AGG GAA TCT GAC GAG GAT
ACT GAG GAT GCT TCT GAG ACC GAC CTC GCG AAA CAT
GAT GAG GAA GAC TAC GTT GAA ATG AAA GAG CAG ATG
TAC CAA GAC AAA CTT GCT AGC CTC AAG AGA CAG TTG
CAG CAA CTG CAA GAA GGC ACG CTC CAG GAG TAC CAG
AAG AGA ATG AAA AAA CTC GAC CAG CAG TAC AAG GAA
CGA ATT AGA AAC GCA GAG CTC TTT CTT CAG CTG GAG
ACT GAA CAG GTT GAG CGC AAT TAT ATT AAG GAA AAA
AAA GCC GCT GTG AAG GAG TTC GAA GAC AAG AAA GTG
GAA CTT AAA GAA AAC CTC ATC GCC GAA CTG GAG GAG
AAG AAG AAG ATG ATA GAG AAC GAA AAA CTC ACA ATG
GAA CTG ACG GGT GAT TCC ATG GAG GTA AAA CCG ATT
ATG ACC CGA AAG CTC CGC CGA CGC CCA AAC GAT CCG
GTA CCG ATC CCT GAT AAG CGG CGC AAG CCC GCA CCG
GCT CAG CTC AAT TAC CTG CTG ACC GAC GAA CAA ATA
ATG GAG GAC CTG CGG ACT CTT AAT AAG CTG AAG AGT
CCT AAA CGG CCA GCT TCC CCC AGT TCC CCC GAA CAC
CTG CCC GCT ACT CCC GCG GAG AGC CCT GCT CAG CGC
TTT GAG GCC CGA ATC GAG GAC GGA AAA TTG TAC TAT
GAC AAA CGC TGG TAT CAT AAG AGC CAG GCT ATA TAC
CTG GAG TCA AAA GAT AAC CAA AAG TTG TCA TGT GTA
ATC TCC TCA GTC GGG GCT AAC GAA ATA TGG GTG CGG
AAG ACC TCT GAT AGT ACG AAG ATG CGC ATA TAT CTG
GGA CAA TTG CAA AGA GGA CTT TTT GTT ATA AGA CGG
AGA AGC GCT GCT.

In some embodiments, the nucleic acid sequence comprises, consists essentially of, or consists of a sequence that is the same as SEQ ID NO: 5.

In some embodiments, the nucleic acid sequence comprises, consists essentially of, or consists of a sequence that is at least 80%, at least 85%, at least 90%, or at least 95% the same as or complementary to SEQ ID NO: 6, which encodes the NIPP1 repressor domain:

ATGGTGCAAACTGCAGTGGTCCCAGTCAAGAAGAAGCGTGTGGAGGGCCC
TGGCTCCCTGGGCCTGGAGGAATCAGGGAGCAGGCGCATGCAGAACTTTG
CCTTCAGCGGAGGACTCTACGGGGGCCTGCCCCCCACACACAGTGAAGCA
GGCTCCCAGCCACATGGCATCCATGGGACAGCACTCATCGGTGGCTTGCC
CATGCCATACCCAAACCTTGCCCCTGATGTGGACTTGACTCCTGTTGTGC
CGTCAGCAGTGAACATGAACCCTGCACCAAACCCTGCAGTCTATAACCCT
GAAGCTGTAAATGAACCCAAGAAGAAGAAATATGCAAAAGAGGCTTGGCC
AGGCAAGAAGCCCACACCTTCCTTGCTGATT.

In some embodiments, the nucleic acid sequence comprises, consists essentially of, or consists of a sequence that is the same as SEQ ID NO: 6.

In some embodiments, the nucleic acid sequence comprises, consists essentially of, or consists of a sequence that is at least 80%, at least 85%, at least 90%, or at least 95% the same as or complementary to SEQ ID NO: 37, which encodes the 5 KRAB repressor domain:

ATGGACGCGAAATCACTTACGGCATGGTCGAGAACACTGGTTACGTTCAA
GGACGTGTTTGTGGACTTTACACGTGAGGAGTGGAAATTGCTGGATACTG
CGCAACAAATTGTGTATCGAAATGTCATGCTTGAGAATTACAAGAACCTC
GTCAGTCTCGGATACCAGTTGACGAAACCGGATGTGATCCTTAGGCTCGA
AAAGGGGGAAGAACCTTGGCTGGTA.

In some embodiments, the nucleic acid sequence comprises, consists essentially of, or consists of a sequence that is the same as SEQ ID NO: 37.

In some embodiments, the nucleic acid sequence comprises, consists essentially of, or consists of a sequence that is at least 80%, at least 85%, at least 90%, or at least 95% the same as or complementary to SEQ ID NO: 38, which encodes the DNMT3A repressor domain:

CCCTCCCGGCTCCAGATGTTCTTCGCTAATAACCACGACCAGGAATTTGA
CCCTCCAAAGGTTTACCCACCTGTCCCAGCTGAGAAGAGGAAGCCCATCC
GGGTGCTGTCTCTCTTTGATGGAATCGCTACAGGGCTCCTGGTGCTGAAG
GACTTGGGCATTCAGGTGGACCGCTACATTGCCTCGGAGGTGTGTGAGGA
CTCCATCACGGTGGGCATGGTGCGGCACCAGGGGAAGATCATGTACGTCG
GGGACGTCCGCAGCGTCACACAGAAGCATATCCAGGAGTGGGGCCCATTC
GATCTGGTGATTGGGGGCAGTCCCTGCAATGACCTCTCCATCGTCAACCC
TGCTCGCAAGGGCCTCTACGAGGGCACTGGCCGGCTCTTCTTTGAGTTCT
ACCGCCTCCTGCATGATGCGCGGCCCAAGGAGGGAGATGATCGCCCCTTC
TTCTGGCTCTTTGAGAATGTGGTGGCCATGGGCGTTAGTGACAAGAGGGA
CATCTCGCGATTTCTCGAGTCCAACCCTGTGATGATTGATGCCAAAGAAG
TGTCAGCTGCACACAGGGCCCGCTACTTCTGGGGTAACCTTCCCGGTATG
AACAGGCCGTTGGCATCCACTGTGAATGATAAGCTGGAGCTGCAGGAGTG
TCTGGAGCATGGCAGGATAGCCAAGTTCAGCAAAGTGAGGACCATTACTA
CGAGGTCAAACTCCATAAAGCAGGGCAAAGACCAGCATTTTCCTGTCTTC
ATGAATGAGAAAGAGGACATCTTATGGTGCACTGAAATGGAAAGGGTATT
TGGTTTCCCAGTCCACTATACTGACGTCTCCAACATGAGCCGCTTGGCGA
GGCAGAGACTGCTGGGCCGGTCATGGAGCGTGCCAGTCATCCGCCACCTC
TTCGCTCCGCTGAAGGAGTATTTTGCGTGTGTG.

In some embodiments, the nucleic acid sequence comprises, consists essentially of, or consists of a sequence that is the same as SEQ ID NO: 38.

In some embodiments, the nucleic acid sequence comprises a sequence that encodes at least one a linker sequence and is at least 80%, at least 85%, at least 90%, or at least 95% the same as or complementary to SEQ ID NO: 8:

GGATCCGGTGGGGGATCTGGGGGATCTGGCTCG.

In some embodiments, the nucleic acid sequence comprises a sequence that is the same as SEQ ID NO: 8.

In some embodiments, the nucleic acid sequence comprises, consists essentially of, or consists of a sequence that is at least 80%, at least 85%, at least 90%, or at least 95% the same as or complementary to SEQ ID NO: 184, which encodes for both the SALL1 and SUDS3 repressor domains:

ATGAGTAGGAGAAAACAAGCAAAACCACAGCACTTTCAAAGTGATCCTGA
GGTAGCAAGCCTTCCACGGCGGGACGGTGACACGGAGAAGGGTCAACCAA
GTCGACCCACGAAAAGCAAAGATGCTCATGTATGTGGACGCTGTTGCGCA
GAATTTTTTGAATTGTCCGATCTTCTTCTTCACAAAAAGAACTGCACGAA
GAATCAGTTGGTTTTGATAGTAAACGAAAATCCAGCTTCACCCCCAGAAA
CTTTTTCCCCGTCACCTCCTCCAGATAATCCTGATGAACAAATGAATGAC
ACCGTAAATAAAACCGACCAAGTAGACTGTTCTGATTTGAGCGAACACAA
CGGTTTGGATCGAGAAGAGTCAATGGAAGTAGAGGCCCCAGTTGCCAATA
AGTCAGGCAGCGGTACTTCTTCCGGCTCCCACAGTTCAACAGCTCCATCC
TCAAGTAGTTCAAGCTCTTCTAGTTCAGGAGGCGGGGGGAGTAGCTCTAC
CGGCACTTCTGCCATCACAACCTCACTTCCTCAGCTTGGAGACTTGACAG
GATCCGGTGGGGGATCTGGGGGATCTGGCTCGATGTCTGCAGCTGGCCTT
TTGGCTCCTGCCCCCGCACAAGCGGGAGCTCCTCCCGCACCGGAGTACTA
TCCAGAAGAGGATGAGGAACTGGAATCTGCCGAAGACGACGAGCGCAGTT
GCCGGGGGAGGGAATCTGACGAGGATACTGAGGATGCTTCTGAGACCGAC
CTCGCGAAACATGATGAGGAAGACTACGTTGAAATGAAAGAGCAGATGTA
CCAAGACAAACTTGCTAGCCTCAAGAGACAGTTGCAGCAACTGCAAGAAG
GCACGCTCCAGGAGTACCAGAAGAGAATGAAAAAACTCGACCAGCAGTAC
AAGGAACGAATTAGAAACGCAGAGCTCTTTCTTCAGCTGGAGACTGAACA
GGTTGAGCGCAATTATATTAAGGAAAAAAAAGCCGCTGTGAAGGAGTTCG
AAGACAAGAAAGTGGAACTTAAAGAAAACCTCATCGCCGAACTGGAGGAG
AAGAAGAAGATGATAGAGAACGAAAAACTCACAATGGAACTGACGGGTGA
TTCCATGGAGGTAAAACCGATTATGACCCGAAAGCTCCGCCGACGCCCAA
ACGATCCGGTACCGATCCCTGATAAGCGGCGCAAGCCCGCACCGGCTCAG
CTCAATTACCTGCTGACCGACGAACAAATAATGGAGGACCTGCGGACTCT
TAATAAGCTGAAGAGTCCTAAACGGCCAGCTTCCCCCAGTTCCCCCGAAC
ACCTGCCCGCTACTCCCGCGGAGAGCCCTGCTCAGCGCTTTGAGGCCCGA
ATCGAGGACGGAAAATTGTACTATGACAAACGCTGGTATCATAAGAGCCA
GGCTATATACCTGGAGTCAAAAGATAACCAAAAGTTGTCATGTGTAATCT
CCTCAGTCGGGGCTAACGAAATATGGGTGCGGAAGACCTCTGATAGTACG
AAGATGCGCATATATCTGGGACAATTGCAAAGAGGACTTTTTGTTATAAG
ACGGAGAAGCGCTGCT.

Additionally or alternatively, in some embodiments, the nucleic acid sequence comprises, consists essentially of, or consists of a sequence that is the same as SEQ ID NO: 183, which encodes deactivated Cas9 (dCas9):

ATGGATTACAAAGACGATGACGATAAGATGGCCCCAAAGAAGAAGCGGA
AGGTCGGTATCCACGGAGTCCCAGCAGCCGACAAGAAGTACAGCATCGG
CCTGGCCATCGGCACCAACTCTGTGGGCTGGGCCGTGATCACCGACGAG
TACAAGGTGCCCAGCAAGAAATTCAAGGTGCTGGGCAACACCGACCGGC
ACAGCATCAAGAAGAACCTGATCGGAGCCCTGCTGTTCGACAGCGGCGA
AACAGCCGAGGCCACCCGGCTGAAGAGAACCGCCAGAAGAAGATACACC
AGACGGAAGAACCGGATCTGCTATCTGCAAGAGATCTTCAGCAACGAGA
TGGCCAAGGTGGACGACAGCTTCTTCCACAGACTGGAAGAGTCCTTCCT
GGTGGAAGAGGATAAGAAGCACGAGCGGCACCCCATCTTCGGCAACATC
GTGGACGAGGTGGCCTACCACGAGAAGTACCCCACCATCTACCACCTGA
GAAAGAAACTGGTGGACAGCACCGACAAGGCCGACCTGCGGCTGATCTA
TCTGGCCCTGGCCCACATGATCAAGTTCCGGGGCCACTTCCTGATCGAG
GGCGACCTGAACCCCGACAACAGCGACGTGGACAAGCTGTTCATCCAGC
TGGTGCAGACCTACAACCAGCTGTTCGAGGAAAACCCCATCAACGCCAG
CGGCGTGGACGCCAAGGCCATCCTGTCTGCCAGACTGAGCAAGAGCAGA
CGGCTGGAAAATCTGATCGCCCAGCTGCCCGGCGAGAAGAAGAATGGCC
TGTTCGGCAACCTGATTGCCCTGAGCCTGGGCCTGACCCCCAACTTCAA
GAGCAACTTCGACCTGGCCGAGGATGCCAAACTGCAGCTGAGCAAGGAC
ACCTACGACGACGACCTGGACAACCTGCTGGCCCAGATCGGCGACCAGT
ACGCCGACCTGTTTCTGGCCGCCAAGAACCTGTCCGACGCCATCCTGCT
GAGCGACATCCTGAGAGTGAACACCGAGATCACCAAGGCCCCCCTGAGC
GCCTCTATGATCAAGAGATACGACGAGCACCACCAGGACCTGACCCTGC
TGAAAGCTCTCGTGCGGCAGCAGCTGCCTGAGAAGTACAAAGAGATTTT
CTTCGACCAGAGCAAGAACGGCTACGCCGGCTACATTGACGGCGGAGCC
AGCCAGGAAGAGTTCTACAAGTTCATCAAGCCCATCCTGGAAAAGATGG
ACGGCACCGAGGAACTGCTCGTGAAGCTGAACAGAGAGGACCTGCTGCG
GAAGCAGCGGACCTTCGACAACGGCAGCATCCCCCACCAGATCCACCTG
GGAGAGCTGCACGCCATTCTGCGGCGGCAGGAAGATTTTTACCCATTCC
TGAAGGACAACCGGGAAAAGATCGAGAAGATCCTGACCTTCCGCATCCC
CTACTACGTGGGCCCTCTGGCCAGGGGAAACAGCAGATTCGCCTGGATG
ACCAGAAAGAGCGAGGAAACCATCACCCCCTGGAACTTCGAGGAAGTGG
TGGACAAGGGCGCTTCCGCCCAGAGCTTCATCGAGCGGATGACCAACTT
CGATAAGAACCTGCCCAACGAGAAGGTGCTGCCCAAGCACAGCCTGCTG
TACGAGTACTTCACCGTGTATAACGAGCTGACCAAAGTGAAATACGTGA
CCGAGGGAATGAGAAAGCCCGCCTTCCTGAGCGGCGAGCAGAAAAAGGC
CATCGTGGACCTGCTGTTCAAGACCAACCGGAAAGTGACCGTGAAGCAG
CTGAAAGAGGACTACTTCAAGAAAATCGAGTGCTTCGACTCCGTGGAAA
TCTCCGGCGTGGAAGATCGGTTCAACGCCTCCCTGGGCACATACCACGA
TCTGCTGAAAATTATCAAGGACAAGGACTTCCTGGACAATGAGGAAAAC
GAGGACATTCTGGAAGATATCGTGCTGACCCTGACACTGTTTGAGGACA
GAGAGATGATCGAGGAACGGCTGAAAACCTATGCCCACCTGTTCGACGA
CAAAGTGATGAAGCAGCTGAAGCGGCGGAGATACACCGGCTGGGGCAGG
CTGAGCCGGAAGCTGATCAACGGCATCCGGGACAAGCAGTCCGGCAAGA
CAATCCTGGATTTCCTGAAGTCCGACGGCTTCGCCAACAGAAACTTCAT
GCAGCTGATCCACGACGACAGCCTGACCTTTAAAGAGGACATCCAGAAA
GCCCAGGTGTCCGGCCAGGGCGATAGCCTGCACGAGCACATTGCCAATC
TGGCCGGCAGCCCCGCCATTAAGAAGGGCATCCTGCAGACAGTGAAGGT
GGTGGACGAGCTCGTGAAAGTGATGGGCCGGCACAAGCCCGAGAACATC
GTGATCGAAATGGCCAGAGAGAACCAGACCACCCAGAAGGGACAGAAGA
ACAGCCGCGAGAGAATGAAGCGGATCGAAGAGGGCATCAAAGAGCTGGG
CAGCCAGATCCTGAAAGAACACCCCGTGGAAAACACCCAGCTGCAGAAC
GAGAAGCTGTACCTGTACTACCTGCAGAATGGGCGGGATATGTACGTGG
ACCAGGAACTGGACATCAACCGGCTGTCCGACTACGATGTGGACGCTAT
CGTGCCTCAGAGCTTTCTGAAGGACGACTCCATCGACAACAAGGTGCTG
ACCAGAAGCGACAAGAACCGGGGCAAGAGCGACAACGTGCCCTCCGAAG
AGGTCGTGAAGAAGATGAAGAACTACTGGCGGCAGCTGCTGAACGCCAA
GCTGATTACCCAGAGAAAGTTCGACAATCTGACCAAGGCCGAGAGAGGC
GGCCTGAGCGAACTGGATAAGGCCGGCTTCATCAAGAGACAGCTGGTGG
AAACCCGGCAGATCACAAAGCACGTGGCACAGATCCTGGACTCCCGGAT
GAACACTAAGTACGACGAGAATGACAAGCTGATCCGGGAAGTGAAAGTG
ATCACCCTGAAGTCCAAGCTGGTGTCCGATTTCCGGAAGGATTTCCAGT
TTTACAAAGTGCGCGAGATCAACAACTACCACCACGCCCACGACGCCTA
CCTGAACGCCGTCGTGGGAACCGCCCTGATCAAAAAGTACCCTAAGCTG
GAAAGCGAGTTCGTGTACGGCGACTACAAGGTGTACGACGTGCGGAAGA
TGATCGCCAAGAGCGAGCAGGAAATCGGCAAGGCTACCGCCAAGTACTT
CTTCTACAGCAACATCATGAACTTTTTCAAGACCGAGATTACCCTGGCC
AACGGCGAGATCCGGAAGCGGCCTCTGATCGAGACAAACGGCGAAACCG
GGGAGATCGTGTGGGATAAGGGCCGGGATTTTGCCACCGTGCGGAAAGT
GCTGAGCATGCCCCAAGTGAATATCGTGAAAAAGACCGAGGTGCAGACA
GGCGGCTTCAGCAAAGAGTCTATCCTGCCCAAGAGGAACAGCGATAAGC
TGATCGCCAGAAAGAAGGACTGGGACCCTAAGAAGTACGGCGGCTTCGA
CAGCCCCACCGTGGCCTATTCTGTGCTGGTGGTGGCCAAAGTGGAAAAG
GGCAAGTCCAAGAAACTGAAGAGTGTGAAAGAGCTGCTGGGGATCACCA
TCATGGAAAGAAGCAGCTTCGAGAAGAATCCCATCGACTTTCTGGAAGC
CAAGGGCTACAAAGAAGTGAAAAAGGACCTGATCATCAAGCTGCCTAAG
TACTCCCTGTTCGAGCTGGAAAACGGCCGGAAGAGAATGCTGGCCTCTG
CCGGCGAACTGCAGAAGGGAAACGAACTGGCCCTGCCCTCCAAATATGT
GAACTTCCTGTACCTGGCCAGCCACTATGAGAAGCTGAAGGGCTCCCCC
GAGGATAATGAGCAGAAACAGCTGTTTGTGGAACAGCACAAGCACTACC
TGGACGAGATCATCGAGCAGATCAGCGAGTTCTCCAAGAGAGTGATCCT
GGCCGACGCTAATCTGGACAAAGTGCTGTCCGCCTACAACAAGCACCGG
GATAAGCCCATCAGAGAGCAGGCCGAGAATATCATCCACCTGTTTACCC
TGACCAATCTGGGAGCCCCTGCCGCCTTCAAGTACTTTGACACCACCAT
CGACCGGAAGAGGTACACCAGCACCAAAGAGGTGCTGGACGCCACCCTG
ATCCACCAGAGCATCACCGGCCTGTACGAGACACGGATCGACCTGTCTC
AGCTGGGAGGCGACAAGCGTCCTGCTGCTACTAAGAAAGCTGGTCAAGC
TAAGAAAAAGAAA

Additionally or alternatively, in some embodiments, the nucleic acid sequence comprises, consists essentially of, or consists of a sequence that is the same as SEQ ID NO: 178, which encodes deactivated MAD7 (dMAD7):

ATGGTCGACGGTAAGCCTATCCCTAACCCTCTCCTCGGTCTCGATTCTA
CGCCAAAAAAGAAGAGAAAGGTCAATAACGGAACTAATAACTTCCAAAA
CTTCATCGGGATCAGTTCCTTGCAGAAAACTCTCCGGAATGCTCTCATC
CCAACTGAGACTACTCAGCAGTTCATTGTTAAGAATGGAATCATAAAAG
AGGACGAGCTTAGGGGGGAAAATAGGCAAATCCTCAAGGATATCATGGA
TGACTATTATAGGGGCTTTATATCCGAGACACTGAGCAGCATTGATGAT
ATAGACTGGACCTCTCTTTTCGAAAAGATGGAAATACAACTTAAAAATG
GAGATAACAAGGACACCCTGATAAAGGAACAGACCGAATATAGGAAGGC
AATTCATAAAAAGTTTGCTAACGATGATAGGTTTAAAAACATGTTCTCA
GCAAAACTCATTTCAGATATACTGCCCGAATTCGTTATCCACAACAACA
ACTACTCCGCTAGCGAAAAAGAGGAAAAGACCCAAGTCATAAAGCTGTT
CTCTCGATTCGCGACGAGTTTTAAAGATTATTTCAAGAATCGCGCAAAC
TGTTTCTCAGCTGATGATATCAGCAGCTCATCCTGTCATCGGATCGTTA
ACGATAATGCTGAAATCTTCTTCTCCAATGCACTTGTTTATAGGCGCAT
TGTTAAATCTCTCTCAAACGATGATATCAATAAGATTTCCGGCGATATG
AAGGACAGTCTTAAGGAGATGAGCCTCGAAGAGATATACTCATACGAGA
AATATGGCGAATTTATCACCCAGGAAGGGATTTCCTTCTATAATGACAT
TTGCGGCAAAGTCAATTCCTTCATGAACCTGTATTGCCAAAAAAATAAA
GAAAACAAGAACCTCTATAAGCTGCAAAAGTTGCATAAGCAAATACTTT
GTATCGCGGATACAAGCTATGAAGTTCCCTACAAGTTCGAGAGTGATGA
GGAGGTGTATCAATCTGTCAATGGTTTCCTTGATAATATTTCTTCTAAG
CATATTGTTGAACGACTCCGAAAGATAGGAGACAACTATAATGGATACA
ATTTGGATAAAATCTACATCGTGTCTAAATTTTACGAGAGTGTGTCACA
AAAAACATATAGAGACTGGGAGACAATTAATACCGCCCTGGAGATACAT
TACAACAATATACTTCCCGGGAACGGGAAGTCTAAGGCAGACAAGGTGA
AGAAAGCCGTGAAGAACGACTTGCAAAAGTCAATTACCGAAATCAATGA
GCTTGTTTCAAACTATAAACTTTGTTCAGATGACAATATTAAAGCCGAA
ACCTATATTCATGAAATCTCTCATATTCTGAATAACTTTGAGGCGCAAG
AACTGAAATATAACCCAGAAATACACCTCGTTGAGTCCGAACTGAAAGC
AAGCGAACTGAAAAATGTTTTGGACGTGATAATGAACGCTTTTCATTGG
TGCTCAGTCTTTATGACAGAGGAGCTTGTTGACAAGGATAACAATTTCT
ATGCGGAACTGGAAGAGATTTACGACGAAATCTATCCGGTCATATCCCT
GTATAACCTGGTTCGCAACTATGTCACGCAAAAACCATACAGCACGAAG
AAGATTAAACTGAACTTTGGTATTCCGACGCTGGCCGATGGATGGTCAA
AATCTAAGGAATACTCAAACAATGCCATAATCCTGATGCGAGATAACCT
CTACTACCTTGGAATCTTTAATGCTAAAAATAAACCCGATAAAAAAATT
ATCGAAGGGAACACGAGTGAAAACAAAGGTGATTATAAAAAAATGATAT
ATAATCTGCTTCCAGGACCAAATAAGATGATACCCAAAGTTTTCCTTTC
TTCAAAGACCGGCGTCGAGACATATAAACCATCCGCGTACATACTTGAA
GGCTACAAACAAAATAAACATATCAAATCATCTAAGGATTTTGACATTA
CGTTCTGTCATGATTTGATTGACTATTTCAAAAATTGCATAGCCATTCA
TCCAGAGTGGAAAAACTTTGGGTTTGACTTCTCTGATACCAGTACATAT
GAAGACATAAGTGGATTTTACCGAGAAGTAGAGCTCCAAGGTTATAAAA
TAGACTGGACCTATATATCTGAAAAGGATATAGACCTTTTGCAAGAGAA
GGGACAGCTTTATCTTTTCCAAATCTACAACAAAGACTTCAGTAAGAAA
AGTACCGGGAATGACAATCTTCATACCATGTATCTGAAGAACCTGTTCT
CCGAAGAAAATCTGAAGGACATAGTCCTGAAGCTTAATGGCGAAGCGGA
AATTTTTTTCCGAAAGAGCTCTATTAAGAACCCCATAATACATAAGAAG
GGAAGCATTCTCGTTAATCGAACGTATGAGGCCGAAGAGAAAGATCAAT
TTGGGAATATCCAAATCGTTCGAAAGAACATACCAGAAAATATTTACCA
AGAATTGTACAAATATTTTAACGATAAAAGCGACAAAGAACTGTCTGAT
GAAGCTGCTAAGCTGAAAAACGTCGTCGGCCATCATGAGGCCGCGACGA
ATATAGTCAAGGATTACCGATATACATACGATAAGTATTTCCTGCATAT
GCCCATCACTATCAACTTTAAGGCAAATAAGACTGGATTCATTAATGAC
AGAATACTGCAATACATAGCTAAAGAAAAAGATTTGCATGTTATTGGCA
TTGCCAGGGGTGAGCGCAATCTTATCTATGTAAGCGTCATTGATACTTG
CGGGAATATCGTAGAGCAGAAGTCATTTAATATTGTAAATGGGTACGAT
TACCAAATCAAGTTGAAGCAGCAAGAGGGAGCACGACAGATTGCCCGCA
AGGAGTGGAAAGAGATCGGAAAGATAAAGGAGATCAAGGAGGGGTATTT
GTCCCTTGTTATACACGAAATTTCCAAGATGGTAATCAAGTACAACGCT
ATAATTGCTATGGCGGATCTCTCCTATGGATTTAAAAAGGGAAGATTTA
AAGTCGAGCGGCAGGTATATCAGAAATTTGAAACAATGCTTATTAATAA
ACTTAATTATCTCGTTTTCAAAGACATTAGTATCACCGAAAACGGTGGG
CTGTTGAAGGGCTATCAACTTACGTACATACCAGATAAGCTTAAGAATG
TGGGTCACCAATGCGGATGCATATTCTACGTGCCCGCAGCTTATACAAG
CAAAATCGACCCAACAACGGGTTTCGTAAACATATTTAAGTTCAAGGAT
CTCACCGTGGATGCCAAGCGAGAGTTCATAAAAAAATTTGACTCAATCA
GATATGACTCAGAAAAGAATCTTTTTTGTTTTACCTTCGACTACAATAA
TTTCATTACACAAAATACGGTTATGAGCAAGTCATCCTGGTCCGTATAT
ACGTATGGAGTGCGCATAAAGCGGAGATTCGTTAACGGGCGATTTTCTA
ATGAGTCCGATACAATCGATATAACAAAGGATATGGAAAAAACTCTGGA
AATGACTGATATAAATTGGAGGGACGGTCATGACCTCAGGCAAGACATT
ATCGATTATGAGATCGTGCAACATATTTTTGAGATCTTTCGGTTGACTG
TCCAAATGAGGAACTCTCTGTCTGAATTGGAAGATAGGGACTACGATCG
CCTGATAAGCCCCGTGTTGAACGAGAATAACATATTCTACGATTCCGCG
AAAGCCGGGGATGCGCTCCCTAAGGACGCCGCTGCAAATGGGGCCTATT
GTATTGCTTTGAAAGGGCTGTACGAAATCAAACAGATCACCGAAAACTG
GAAAGAAGACGGGAAGTTTAGTCGGGATAAACTGAAGATATCCAACAAG
GACTGGTTTGACTTTATCCAAAATAAGCGATATTTGAAGCGTCCTGCTG
CTACTAAGAAAGCTGGTCAAGCTAAGAAAAAGAAA

In some embodiments, the nucleic acid sequence comprises, consists essentially of, or consists of a sequence that is at least 80%, at least 85%, at least 90%, or at least 95% the same as or complementary to SEQ ID NO: 178.

Additionally or alternatively, in some embodiments, the nucleic acid sequence comprises, consists essentially of, or consists of a sequence that is the same as SEQ ID NO: 179, which encodes deactivated CasPhi8 (dCasPhi8):

ATGGTCGACGGGAGCGGGCCGGCAGCTAAACGGGTGAAGTTGGACAGTGG
TGGAATTAAACCTACAGTTTCTCAGTTTCTTACCCCTGGTTTTAAGCTGA
TAAGAAACCATAGTCGGACGGCTGGACTTAAGCTGAAGAATGAGGGCGAA
GAGGCATGCAAGAAGTTCGTACGGGAGAACGAAATTCCCAAAGATGAATG
TCCAAACTTTCAAGGTGGACCCGCAATCGCGAACATTATAGCCAAGAGTC
GCGAATTTACCGAGTGGGAAATATATCAAAGTTCACTGGCGATCCAAGAG
GTGATTTTCACCTTGCCGAAGGATAAGCTGCCCGAGCCTATACTCAAGGA
AGAATGGCGCGCCCAATGGTTGAGCGAACACGGCCTCGATACGGTGCCTT
ACAAGGAAGCTGCCGGACTTAATTTGATAATTAAGAACGCGGTCAACACT
TACAAAGGGGTCCAGGTGAAAGTCGATAATAAGAATAAGAACAACCTGGC
CAAAATCAACCGCAAGAACGAAATCGCGAAATTGAACGGCGAACAAGAAA
TCAGCTTCGAAGAGATCAAAGCCTTCGATGATAAAGGATATCTCCTGCAA
AAGCCAAGTCCGAATAAGAGCATATATTGCTACCAAAGCGTGTCTCCAAA
GCCATTCATAACCTCTAAATACCATAACGTGAATCTGCCCGAAGAATATA
TCGGCTACTACCGCAAGTCAAACGAGCCCATCGTTAGTCCCTATCAATTC
GATAGATTGCGAATCCCAATTGGCGAACCCGGATATGTACCAAAATGGCA
GTATACCTTTCTGTCTAAGAAAGAGAATAAGCGGAGAAAGCTCTCCAAGC
GGATTAAGAATGTTAGTCCTATTCTTGGGATAATATGCATTAAGAAAGAC
TGGTGCGTATTCGATATGAGGGGCCTGCTCAGAACGAACCACTGGAAGAA
ATACCATAAACCGACAGATTCTATCAATGACCTCTTCGATTATTTCACTG
GAGACCCTGTAATCGACACGAAAGCGAACGTCGTCCGATTCAGATATAAA
ATGGAAAATGGCATTGTTAATTACAAGCCGGTGCGCGAAAAGAAAGGCAA
GGAACTTTTGGAAAACATATGTGATCAAAATGGGAGCTGTAAGTTGGCCA
CTGTGGCCGTTGGTCAAAACAACCCAGTGGCAATTGGACTGTTTGAACTT
AAGAAAGTAAATGGTGAACTTACCAAAACCTTGATTTCACGGCATCCTAC
TCCGATCGACTTTTGTAATAAAATTACGGCTTACAGGGAGCGGTATGATA
AGCTCGAATCCAGCATCAAGTTGGATGCCATAAAGCAATTGACATCTGAG
CAAAAGATCGAAGTTGATAACTATAACAATAATTTTACCCCTCAAAACAC
TAAGCAGATAGTGTGCAGCAAGCTCAATATCAATCCAAACGACCTTCCTT
GGGATAAAATGATTTCTGGGACTCATTTCATTAGCGAGAAAGCCCAAGTC
AGTAATAAATCAGAAATATACTTCACATCTACCGATAAGGGGAAAACTAA
GGACGTAATGAAGAGCGACTACAAGTGGTTTCAAGACTATAAACCAAAAC
TGTCAAAGGAAGTAAGGGACGCACTCAGCGATATTGAATGGCGGCTTAGG
AGAGAAAGTCTTGAATTTAACAAATTGAGTAAATCACGGGAACAAGATGC
ACGGCAACTGGCCAATTGGATCTCTTCCATGTGTGATGTTATCGGAATAG
AGAACCTGGTGAAGAAGAACAATTTCTTTGGTGGAAGCGGCAAGAGGGAA
CCGGGGTGGGACAACTTCTATAAACCGAAGAAGGAGAATCGATGGTGGAT
CAACGCAATTCATAAAGCTCTCACAGAACTCTCTCAAAACAAAGGGAAAA
GAGTGATTCTCTTGCCAGCAATGAGAACATCTATCACATGCCCTAAATGT
AAGTACTGTGACAGCAAGAACCGGAACGGCGAGAAGTTCAATTGTCTGAA
GTGTGGCATAGAACTCAACGCAGACATTGATGTTGCTACCGAAAATCTCG
CGACCGTTGCTATTACCGCGCAAAGTATGCCTAAACCCACCTGTGAGAGG
AGTGGTGATGCCAAGAAGCCCGTACGTGCACGAAAGGCAAAGGCGCCAGA
ATTTCATGACAAACTCGCGCCCTCATACACAGTTGTCTTGCGCGAAGCTG
TTAAGCGTCCTGCTGCTACTAAGAAAGCTGGTCAAGCTAAGAAAAAGAAA

In some embodiments, the nucleic acid sequence comprises, consists essentially of, or consists of a sequence that is at least 80%, at least 85%, at least 90%, or at least 95% the same as or complementary to SEQ ID NO: 179.

In some embodiments, the fusion protein of the present invention may be linked to nuclear localization signals (NLS), epitope tags, or reporter gene sequences. Examples of nuclear localization signals include, but are not limited to, those of the SV40 Large T-antigen, nucleoplasmin, EGL-13, and TUS-protein. Examples of epitope tags include, but are not limited to, FLAG tags, V5 tags, histidine (His) tags, and influenza hemagglutinin (HA) tags. Examples of reporter genes include, but are not limited to, green fluorescent protein (GFP), red fluorescent protein (RFP), small ubiquitin-like modifier (SUMO), ubiquitin, glutathione-S-transferase (GST), horseradish peroxidase (HRP), chloramphenicol acetyltransferase (CAT), and luciferase.

In some embodiments, the nucleic acid or vector that encodes the fusion proteins of present invention will also encode various regulatory elements or selection markers. Regulatory elements include, but are not limited to promoters such as the cytomegalovirus (CMV) promoter or human EF1α promoter, enhancers such as the woodchuck hepatitis post-transcriptional regulatory element (WPRE) or HIV-1 Rev response element (RRE), polyadenylation signals, self-cleaving peptides such as T2A, and internal ribosomal entry sites (IRES). Examples of selection markers include, but are not limited to, green fluorescent protein (GFP), red fluorescent protein (RFP), puromycin N-acetyl-transferase (PAC) conferring resistance to puromycin, the hygromycin resistance gene, and blasticidin-S deaminase (BSD).

### Methods of Modulating Expression

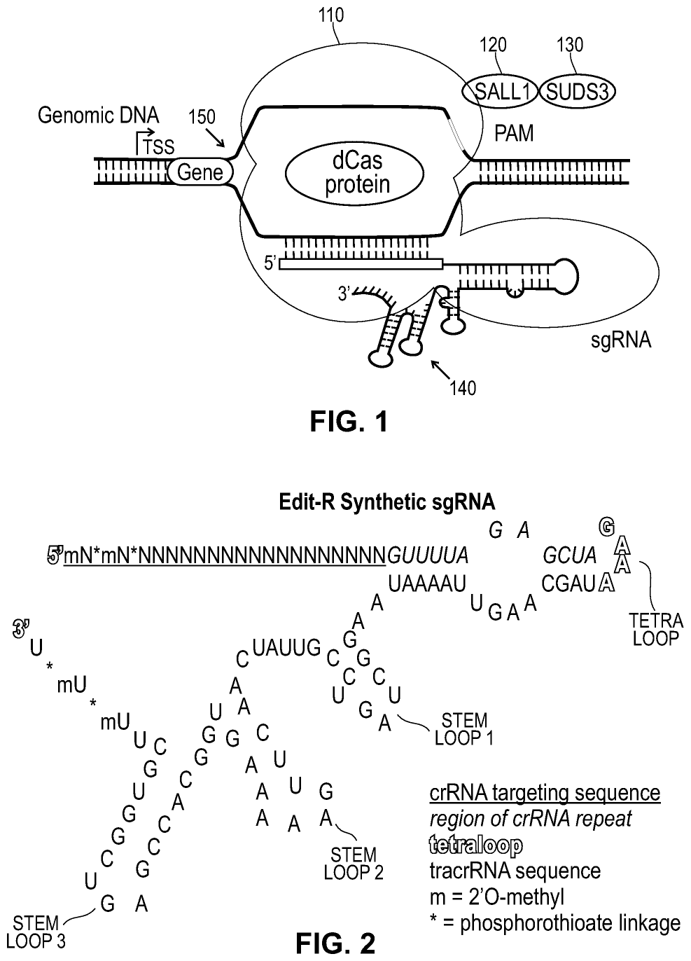

In some embodiments, the present invention is directed to a method of modulating expression of a target nucleic acid in a eukaryotic cell. The method comprises providing to the cell a gRNA and a Cas fusion protein of any of the embodiments of the present invention. When associated with a gRNA as shown in FIG. 1, the Cas protein (shown as a dCas protein) 110 that is fused to SALL1 120, which is fused to SUDS3 130 may act upon a target region of genomic DNA 150.

In some embodiments, the method comprises introduce a plurality of gRNAs with the Cas fusion protein. The plurality of gRNAs may be two or more, e.g., 2-10 or 4-8 gRNAs.

Two or more or all of the gRNAs may target the same gene or the same locus within a gene. If two or more gRNAs target the same locus, they may have the same or overlapping spacer sequences or non-overlapping sequences. In some embodiments, two or more or all of the gRNAs may target the different genes or the different loci within a gene.

In some embodiments, one or more gRNAs are provided to a cell by introducing to the cell a nucleic acid encoding the gRNA, and the Cas fusion protein is provided to the cell by introducing to the cell a nucleic acid encoding the Cas fusion protein. The cell may be placed under conditions in which the cell expresses the gRNA and the Cas fusion protein.

In some embodiments, the present invention is directed to a method of modulating expression of a target nucleic acid in a eukaryotic cell by introduce a Cas fusion protein and an RNA-repressor domain complex. In some embodiments, the present invention is directed to a method of modulating expression of a target nucleic acid in a eukaryotic cell by introduce a Cas protein that is not a fusion protein and an RNA-repressor domain complex.

In some embodiments, the eukaryotic cell is a yeast cell, a plant cell or a mammalian cell such as a human or murine cell. In some embodiments, the cell is part of a cell line, e.g., HEK293, K562, Jurkat, or US2OS.

When a fusion protein is introduced, the fusion protein may be synthesized outside of a cell or an organism. Alternatively, one may introduce an mRNA that encodes the fusion protein. In some embodiments, a gRNA is synthetically made outside of the cell and a Cas fusion protein is provided to the cell by introducing to the cell a nucleic acid encoding the Cas fusion protein.

The Cas fusion proteins, RNA-repressor domain complexes and/or gRNAs may be delivered to target cells and organisms via other various methods and various formats (DNA, RNA or protein) or combination of these different formats. For example, different components may be delivered as: (a) DNA polynucleotides that encode the relevant sequence for the Cas fusion protein or the gRNAs; (b) RNA encoding the sequence for the Cas fusion protein (messenger RNA) and synthetic gRNAs: (c) purified protein for the Cas fusion protein; (d) RNA that encode gRNA; and (e) purified RNA-repressor domain complexes.

When delivering a Cas fusion protein in a protein format, the Cas protein can be assembled with the applicable gRNA to form a ribonucleoprotein complex (RNP) for delivery into target cells, organisms and subjects. For example, the components or complexes ([Cas fusion protein]-[gRNA]) as assembled may be delivered together or separately by electroporation, by nucleofection, by transfection, via nanoparticles, via viral mediated RNA delivery, via non-viral mediated delivery, via extracellular vesicles (for example, exosome and microvesicles), via eukaryotic cell transfer (for example, by recombinant yeast) and other methods that can package molecules such that they can be delivered to a target viable cell without changes to the genomic landscape. Other methods include, but are not limited to, non-integrative transient transfer of DNA polynucleotides that include the relevant sequence for the protein recruitment so that the molecule can be transcribed into the desired RNA molecule. This includes, without limitation, DNA-only vehicles (for example, plasmids, MiniCircles, MiniVectors, MiniStrings, Protelomerase generated DNA molecules (for example, Doggybones), artificial chromosome (for example HAC), and cosmids), via DNA vehicles by nanoparticles, extracellular vesicles (for example, exosome and microvesicles), via eukaryotic cell transfer (for example, by recombinant yeast), transient viral transfer by AAV, non-integrating viral particles (for example, lentivirus and retrovirus based systems), cell penetrating peptides and other technology that can mediate the introduction of DNA into a cell without direct integration into the genomic landscape.

Another method for the introduction of the RNA components include the use of integrative gene transfer technology for stable introduction of the machinery for RNA transcription into the genome of the target cells. These methods can be controlled via constitutive or promoter inducible systems to attenuate the RNA expression and this can also be designed so that the system can be removed after the utility has been met (for example, introducing a Cre-Lox recombination system), such technology for stable gene transfer includes, but is not limited to, integrating viral particles (for example lentivirus, adenovirus and retrovirus based systems), transposase mediate transfer (for example, Sleeping Beauty and Piggybac), exploitation of the non-homologous repair pathways introduced by DNA breaks (for example, utilizing CRISPR and TALEN) technology and a surrogate DNA molecule, and other technology that encourages integration of the target DNA into a cell of interest.

The various components of the complexes of the present invention, if not synthesized enzymatically within a cell or solution, may be created chemically or, if naturally occurring, isolated and purified from naturally occurring sources. Methods for chemically and enzymatically synthesizing the various embodiments of the present invention are well known to persons of ordinary skill in the art. Similarly, methods for ligating or introducing covalent bonds between components of the present invention are also well known to persons of ordinary skill in the art.

### Kits

In some embodiments, the present invention is directed to a kit that comprises, consists essentially of, or consists of a Cas fusion protein of the present invention or a polynucleotide with a nucleic acid sequence that encodes a protein of the present invention. In some embodiments, the kit further comprises a gRNA or a nucleic acid that encodes a gRNA or a plurality of gRNAs or a library of gRNAs, and optionally reagents for transfection and/or other delivery into a cell or to a subject. In some embodiments, the kit comprises a nucleic acid that is capable of expressing both a gRNA and a Cas fusion protein of the present invention. In some embodiments, the kit comprises a cell line that has been engineered to express a Cas fusion protein of the present invention and optionally further comprises a gRNA or a nucleic acid that encodes a gRNA.

In some embodiments, the present invention is directed to a kit that comprises, consists essentially of, or consists of an RNA-repressor domain complex of the present invention. In some embodiments, the kit further comprises a Cas protein or a Cas fusion protein or a nucleic acid that encodes a Cas protein or a Cas fusion protein, and optionally reagents for transfection and/or other delivery into a cell or to a subject.

In one embodiment, the present invention provides a kit, wherein the kit comprises: (1) a lentiviral particle, wherein the lentiviral particle comprises a first polynucleotide that encodes a Cas fusion protein of the present invention, such as dCas9-SALL1-SUDS3; and (2) a second polynucleotide, wherein the second polynucleotide is an sgRNA.

In another embodiment, the present invention provides a kit, wherein the kit comprises: (1) a first lentiviral particle, wherein the first lentiviral particle comprises a first polynucleotide that encodes a Cas fusion protein of the present invention, such as dCas9-SALL1-SUDS3; and (2) a second lentiviral particle, wherein the second lentiviral particle comprises a second polynucleotide, wherein the second polynucleotide codes for an sgRNA.

In another embodiment, the present invention provides a kit, wherein the kit comprises: (1) a lentiviral particle, wherein the lentiviral particle comprises a first polynucleotide that encodes a Cas fusion protein of the present invention, such as dCas9-SALL1-SUDS3; and (2) a second polynucleotide, wherein the second polynucleotide is a plasmid, wherein the plasmid encodes a second polynucleotide and the second polynucleotide is an sgRNA.

In another embodiment, the present invention provides a kit, wherein the kit comprises: (1) a first polynucleotide, wherein the first polynucleotide is an mRNA that encodes a Cas fusion protein of the present invention, such as dCas9-SALL1-SUDS3; and (2) a second polynucleotide, wherein the second polynucleotide is an sgRNA.

The sgRNAs in the kits may be designed to associate with the Cas fusion protein that is encoded by the polynucleotides described above. Optionally, within the kits may be one or more of the following: target cells, and one or more a selection chemicals and/or media (e.g., blasticidin, puromycin).

### Applications

In another embodiment the present invention provides method for simultaneous repression of multiple genes. In some of these methods one may deliver the same dCas9-repressor Cas fusion protein with different gRNAs that target different gene promoters or different transcriptional start sites of the same gene. In another embodiment, the present invention provides a method for simultaneous repression and gene editing. In these methods one may deliver the Cas9-repressor Cas fusion protein with regular gRNAs (20 nucleotide targeting region) to cause gene editing and truncated gRNAs (14 nucleotide targeting region) to cause gene repression. These methods may be used to repress an inflammatory response such as the myeloid differentiation primary response 88 (MyD88) while performing gene editing, or to repress various genes involved in non-homologous end-joining thereby increasing the likelihood of a homology-directed DNA repair event (HDR) or to modulate host genes that are involved in the regulation of repair of double-stranded DNA breaks, leading to different outcomes. These methods may be used to effect synthetic lethality whereby a gene target can be edited and a secondary gene target can be repressed to cause a cytotoxic response not present in cells containing only one of the genomic perturbations.

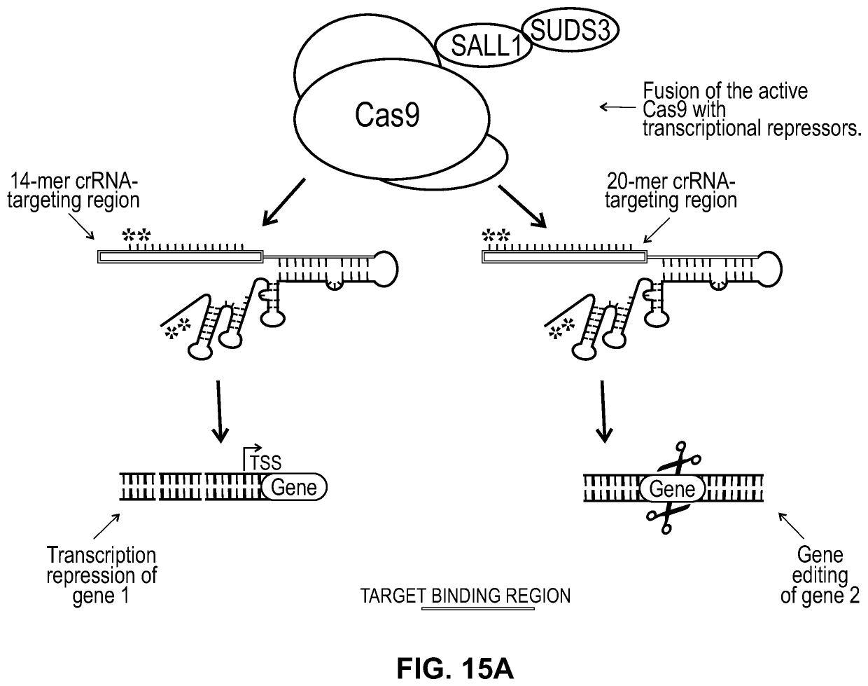

FIG. 15 illustrates the effect of using different sized crRNA regions with an active Cas9 that is fused to SALL1 and SUDS3. When a 14-mer crRNA targeting region is used, there is transcriptional repression of the target (left y-axis of figure). Whereas, when a 20-mer crRNA targeting region is used, there is gene-editing of the target (right y-axis of figure).

The various embodiments of the present invention may also be used in arrayed screening applications. For example, one may use a library of arrayed gRNAs for systematic loss-of-function studies. In some embodiments 2-5 synthetic guide RNAs can be pooled for arrayed screening applications.

The various embodiments of the present invention may also be used in pooled lentiviral screening applications. For example, one may use a pooled library of lentiviral sgRNA constructs targeting a set of gene targets or the whole genome for systematic loss-of-function studies. These gRNAs can be delivered in cells expressing the Cas fusion constructs of the present invention, or via a lentiviral construct that expresses both the Cas fusion protein and a gRNA.

Additionally, in other embodiments, one may combine different CRISPR Cas systems with different effectors in the same cells to cause transcriptional repression with one system and another effect (activation, gene editing, base editing, or epigenetic modification) with the other Cas system.

These methods may, for example, be used to cause specific gene repression of an immune cell selected from a T cell (including a primary T cell), Natural Killer (NK cell), B cell, or CD34+ hematopoietic stem progenitor cell (HSPC). The immune cell may be an engineered immune cell, such as T-cell comprising a chimeric antigen receptor (CAR) or an engineered T cell receptor (TCR). The methods herein may thus be applied to further modulate gene expression of a cell that has already been modified to include a CAR and/or TCR that is useful in therapy. By way of further example, primary immune cells, either naturally occurring within a host animal or patient, or derived from a stem cell or an induced pluripotent stem cell (iPSC) may be used for specific gene repression using the methods and complexes provided herein.

Suitable stem cells include, but are not limited to, mammalian stem cells such as human stem cells, including, but not limited to, hematopoietic, neural, embryonic, iPSC, mesenchymal, mesodermal, liver, pancreatic, muscle, and retinal stem cells. Other stems cells include, but are not limited to, mammalian stem cells such as mouse stem cells, e.g., mouse embryonic stem cells.

Also provided herein are methods for genome engineering (e.g., altering or manipulating the expression of one or more genes or one or more gene products) in prokaryotic or eukaryotic cells, in vitro, in vivo, or ex vivo. In particular, the methods provided herein may be useful for targeted gene expression modulation in mammalian cells including primary human T cells, NK cells, CD34+ HSPCs, such as HSPCs isolated from umbilical cord blood or bone marrow and cells differentiated from them.

Also provided herein are genetically engineered cells arising from haematopoietic stem cells, such as T cells, that have been modified according to the methods described herein.

By way of a non-limiting example, the various embodiments of the present invention may be used for the following applications, base editing, genome editing, genome screening, generation of therapeutic cells, genome tagging, epigenome editing, karyotype engineering, chromatin imaging, transcriptome and metabolic pathway engineering, genetic circuits engineering, cell signaling sensing, cellular events recording, lineage information reconstruction, gene drive, DNA genotyping, miRNA quantification, in vivo cloning, site-directed mutagenesis, genomic diversification, and proteomic analysis in situ. In some embodiments, a cell or a population of cells are exposed to a fusion protein of the present invention and the cell or cells are introduced to a subject by infusion.

Applications also include research of human diseases such as cancer immunotherapy, antiviral therapy, bacteriophage therapy, cancer diagnosis, pathogen screening, microbiota remodeling, stem-cell reprogramming, immunogenomic engineering, vaccine development, and antibody production.

In some embodiments, one or more molecules or complexes descried herein, including a Cas fusion protein, a fusion protein, a Cas protein, a gRNA, and a nucleic acid that encodes any of the foregoing is introduced to a subject. Introduction may, for example, be in the form of a medicament.

### EXAMPLES

In the examples below, applicable protocols from the following methods and materials were used:

### Stable Cell Line Generation

U2OS Ubi[G76V]-EGFP (BioImage, discontinued), U2OS (ATCC, cat #HTB-96), A375 (ATCC, cat #CRL-161, K-562 (ATCC, cat #CCL-243), Jurkat (ATCC, cat #TIB-152), HCT 116 (ATCC, cat #CCL-247), and WTC-11 hiPS (Coriell institute, cat #GM25256) cells were transduced at a multiplicity of infection (MOI) of 0.3 with lentiviral particles co-expressing the blasticidin resistance gene and various Cas-based effector proteins designed for CRISPRi. Cells were subsequently cultured in cell-line-specific medium containing 5-10 μg/mL blasticidin for a minimum of ten days to select cell populations that stably expressed the CRISPRi effector proteins. U2OS cells stably expressing dCas9 were subsequently transduced at an MOI of 0.3 with lentiviral particles that co-expressed MCP-SALL1-SUDS3 and the hygromycin resistance gene. The cells were cultured for 14 days in medium containing 200 μg/mL hygromycin to select for a population that stably expressed both MCP-SALL1-SUDS3 and dCas9.

### Synthesis of Guide RNAs

All sgRNA, crRNA and tracrRNA were synthesized at Horizon Discovery (formerly Dharmacon), sgRNA and crRNA molecules were designed based on the CRISPRi version 2.1 (v 2.1) guide RNA prediction algorithm developed in 2016, M. A. Horlbeck et al., “Compact and highly active next-generation libraries for CRISPR-mediated gene repression and activation,” eLife. 5, e19760 (2016). Unless otherwise stated, experiments utilized modified sgRNAs delivered as an equimolar pool of the top three algorithmically ranked sgRNAs, labeled g1-g3 in table 2 below. The same targeting sequences were used for the sgRNA, crRNA, and expressed sgRNA with the exception that the first base in the expressed sgRNAs is always G.

Lipid Transfections with Synthetic Guide RNAs

U2OS or A375 cells were seeded in 96-well plates at 10,000 or 20,000 cells per well, respectively, one day prior to transfection. Cells were transfected with synthetic guide RNAs targeting specific genes at a final concentration of 25 nM. Synthetic guide RNAs were complexed with DharmaFECT 4 Transfection Reagent (Horizon Discovery, cat #T-2005-01) for each experiment in serum-free medium (GE Healthcare HyClone, cat #SH30564.01) for 20 minutes. Medium on the plated cells was removed and replaced with the transfection mixture. The cells were incubated at 37° C. with 5% CO₂ for 24-144 hours until the assays were performed.

Co-Transfections with dCas9 mRNA and Synthetic sgRNA

U2OS cells were seeded at 10,000 cells per well in clear 96-well plates one day prior to transfection; HCT 116 cells were seeded at 200,000 cells per well in clear 6-well plates one day prior to transfection. U2OS cells were co-transfected with 0.2 μg/well of dCas9-SALL1-SUDS3 or dCas9-KRAB mRNA and 25 nM synthetic sgRNA; HCT 116 cells were co-transfected with 2.5 μg/well of dCas9-SALL1-SUDS3 or dCas9-KRAB mRNA and 25 nM synthetic sgRNA. dCas9 mRNA and sgRNAs were complexed with DharmaFECT Duo Transfection Reagent (Horizon Discovery, cat #T-2010) in serum-free medium (GE Healthcare HyClone, cat #SH30564.01) for 20 minutes. Medium on the plated cells was removed and replaced with the transfection mixture. The cells were incubated at 37° C. with 5% CO₂.

### Nucleofection

K562, Jurkat, WTC-11 human induced pluripotent stem cells (hiPS cells), and primary human CD4+ T cells were electroporated per well using the Amaxa 96-well Shuttle System. 200,000 K562 cells per replicate were resuspended in SF buffer (Lonza, cat #V4SC-2096) and nucleofected using the FF-120 program; 200,000 Jurkat cells were resuspended in SE buffer (Lonza, cat #V4SC-1960) and nucleofected using program C1-120; 80,000 hiPS cells were resuspended in P3 buffer (Lonza, cat #V4SP-3096) and nucleofected using program DC-100; 250,000 primary human CD4+ T cells were resuspended in P3 buffer and nucleofected using program E0-115. Synthetic guide RNAs were delivered at cell-line-dependent final concentrations between 2.5 and 9 μM. In cases where the cells were not stably expressing a dCas9 CRISPRi construct, dCas9-SALL1-SUDS3 or dCas9-KRAB mRNA was delivered at cell-line-dependent concentrations ranging from 1-2.5 μg per nucleofection.

Transfections with Plasmid sgRNA

U2OS and A375 cells were seeded in 96-well plates at 10,000 or 20,000 cells per well one day prior to transfection with CRISPRi sgRNA plasmids. Plasmids were complexed with DharmaFECT kb Transfection Reagent (Horizon Discovery, Cat #T-2006) in serum-free medium (GE Healthcare HyClone, #SH30564.01) for 10 minutes. Medium on the plated cells was removed and replaced with the transfection mixture. The cells were incubated at 37° C. with 5% CO₂ for 72 hours until the assays were performed.

### Lentiviral Transduction

U2OS and HCT 116 cells were seeded at 10,000 cells per well and transduced with CRISPRi sgRNA lentiviral particles at a multiplicity of infection (MOI) of 0.3 to obtain cells with a single integrant. Cells were selected with 2.5 μg/mL puromycin for 7 days with passaging every 3-4 days prior to RT-qPCR analysis.

### RT-qPCR

Total RNA was isolated, reverse-transcribed using Maxima First Strand cDNA Synthesis Kit for RT-qPCR, with dsDNase (ThermoFisher Scientific, cat #K1672) and assessed with qPCR using TaqMan Gene Expression Master Mix and TaqMan Gene Expression Assays. The relative expression of each gene was calculated with the ΔΔCq method using GAPDH or ACTB as the housekeeping gene and normalized to a non-targeting control (NTC).

### Proteasome Assay—a Functional Reporter Assay for Proteasome Gene Inhibition

The proteasome assay utilizes a recombinant U2OS cell line that stably expresses a mutant Ubiquitin fused to enhanced green fluorescent protein (Ubi[G76V]-EGFP). At the experimental endpoint, cell media was replaced with Dulbecco's Phosphate Buffered Saline (Cytivia, cat #SH30028.02) and EGFP fluorescence was measured using an EnVision® plate reader. Fluorescent values of cell populations transfected with guide RNAs targeting critical proteasome genes were normalized to fluorescent values of the untreated cell populations.

### Sanger Sequencing Gene Editing Analysis

Cells were lysed in 100 μL of a buffer containing proteinase K (Thermo Scientific, #FEREO0492), RNase A (Thermo Scientific, #FEREN0531), and Phusion HF buffer (Thermo Scientific, #F-518L) for 30 min at 56° C., followed by a 5 minute heat inactivation at 95° C. This cell lysate was used to generate 400-600 nucleotide PCR amplicons spanning the region containing the gene editing site(s). Unpurified PCR amplicons were subjected to Sanger sequencing. Gene editing efficiencies were calculated from AB1 files using TIDE analysis, Brinkman et al., “Easy quantitative assessment of genome editing by sequence trace decomposition,” Nucleic acids research, 42(22) (2014). TIDE quantifies the frequency and types of small insertions and deletions (indels) at a target locus using quantitative sequence trace data from a targeted sample that is normalized to sequence trace data of a control sample.

### FACS Analysis

24 and 72 hours post-nucleofection, functional knockdown of CXCR3 was assessed as a percent of cells expressing the target gene by FACS analysis. Cells were resuspended in a 1:50 solution of Fc block (BD Biosciences, cat #564220) and stained for CD4 as a positive expression control using an Alexa Fluor 488 conjugated antibody (Biolegend, cat #50166932) and CXCR3 using an APC conjugated primary antibody (Biolegend, cat #353707). Unstained cells were used to gate for CD4 and CXCR3 positive cells. The percent CXCR3 positive cells in the targeted populations was normalized to that in the control populations to determine functional knockdown.

### Example 1: Comparison of Silencing of dCas9-KRAB and dCas9-SALL1-SUDS3 Delivered as mRNA

A comparison of silencing by dCas9-KRAB and dCas9-SALL1-SUDS3 was undertaken against each of the following targets: BRCA1, PPIB, CD46, PSMD7, SEL1L, and ST3GAL4. K562 cells were nucleofected with either dCas9-KRAB or dCas9-SALL1-SUDS3 mRNA and gene knockdown was then measured. In each case, the cells were supplied with a 5 μM mixture of a pool of three pooled synthetic sgRNAs targeting the respective gene.

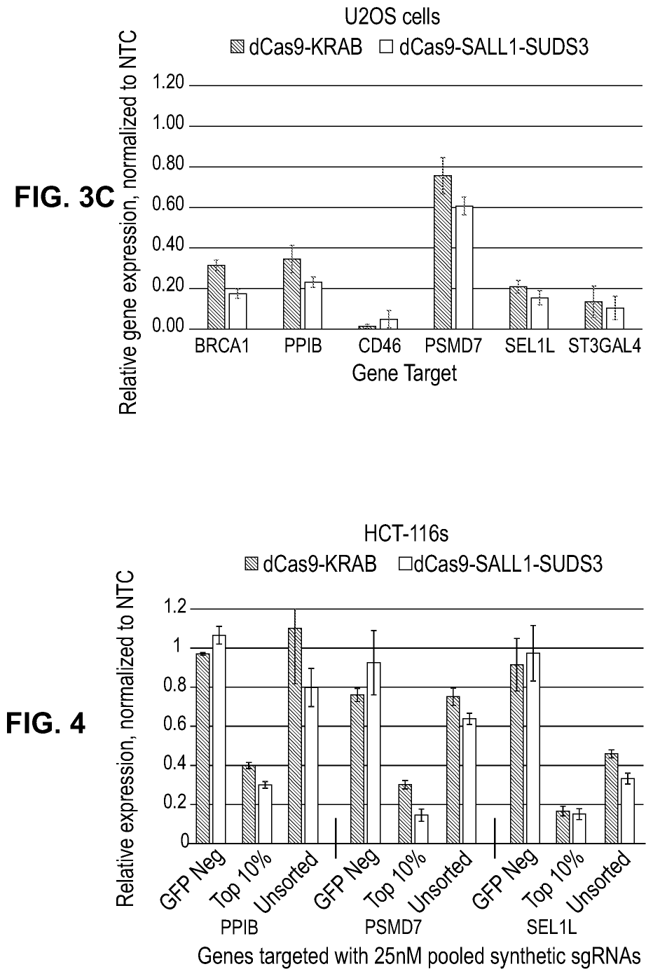

Forty-eight hours after nucleofection, gene-expression was measured relative to a non-targeting control. The results appear in FIG. 3A. As the bar graph shows, in each instance, silencing of gene expression by dCas9-SALL1-SUDS3 was comparable to, if not better than, silencing by dCas9-KRAB.

FIG. 3B shows the results of a similar study, except that the target cells were Jurkat cells and the expression was measured 72 hours after nucleofection. In FIG. 3B, one again sees that in each instance, silencing of gene expression by dCas9-SALL1-SUDS3 was comparable to, if not better than, silencing by dCas9-KRAB.

FIG. 3C shows the results of another similar study, except that the target cells were U2OS cells, reagents were delivered via lipid transfection using a 25 nM mixture of a pool of three pooled synthetic sgRNAs targeting the respective gene, and the expression was measured 72 hours after transfection. In FIG. 3C, one again sees that in each instance, silencing of gene expression by dCas9-SALL1-SUDS3 was comparable to, if not better than, silencing by dCas9-KRAB.

### Example 2: Comparison of Effectiveness of Cas Fusion Protein Repressor to dCas9-KRAB

HCT116 cells were plated at 400,000 cells per well. Twenty-four hours later, the cells were co-transfected with dCas9-SALL1-SUDS3 eGFP mRNA or dCas9-KRAB eGFP mRNA and a 25 nM mixture of a pool of three synthetic sgRNAs targeting each of the following genes: PPIB, PSMD7, and SEL1L, as well as a nontargeting control (NTC), using DharmaFECT® Duo Transfection reagent. At 24 hours post-transfection, cells were trypsinized, and FACS was performed. Cells were sorted into two categories: Negative, and Top 10%, then plated in 6-well dishes and allowed to recover. After 24 hours of recovery (48 hours total), the total amount of RNA was isolated and relative gene expression was measured using RT-qPCR. The relative expression of each gene was calculated with the ΔΔCq method using GAPDH as the housekeeping gene and normalized to a non-targeting control.

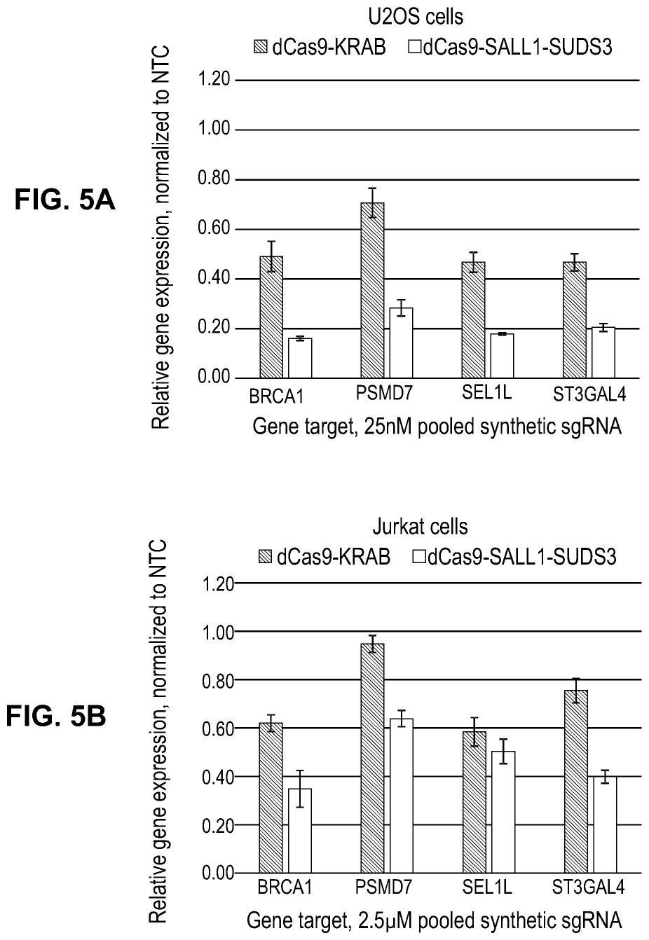

As FIG. 4 shows, dCas9-SALL1-SUDS3 eGFP mRNA can be used for FACS enrichment and provides greater repression of target genes than dCas9-KRAB eGFP mRNA in both selected and unselected populations.

### Example 3: Comparison Repression of dCas9-KRAB to dCas9-SALL1-SUDS3 in Different Cell Lines

U2OS, Jurkat, and hiPS cells stably expressing dCas9-SALL1-SUDS3 or dCas9-KRAB were transfected or nucleofected with pools of three synthetic sgRNAs targeting the listed genes, as well as NTCs. Cells were harvested 72 hours later. In each cell line dCas9-KRAB or dCas9-SALL1-SUDS3 were under control of the hEF1α promoter. The total RNA was isolated and relative gene expression was measured using RT-qPCR. The relative expression of each gene was calculated with the ΔΔCq method using GAPDH as the housekeeping gene and normalized to a non-targeting control.

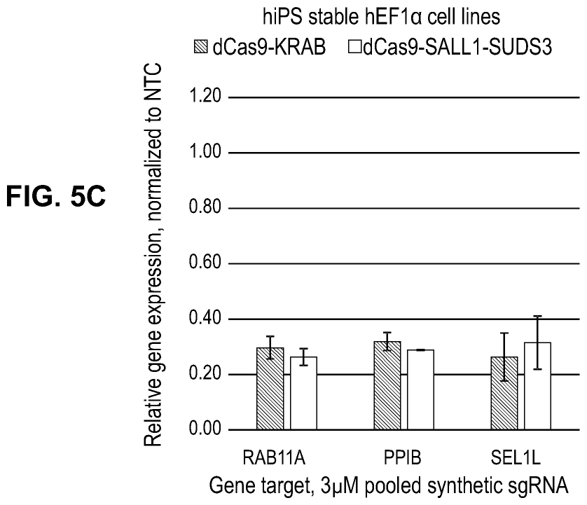

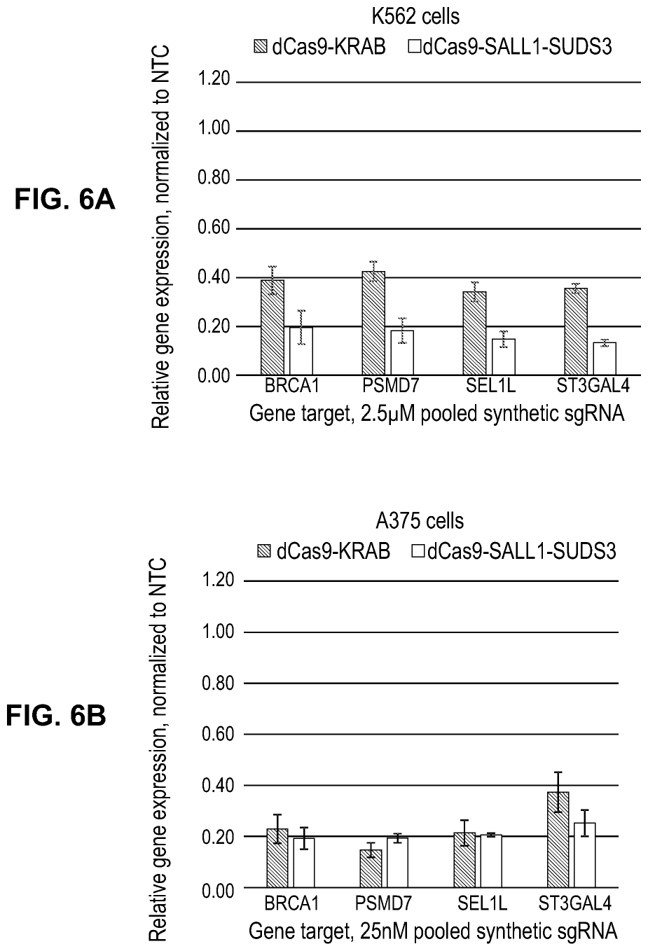

As FIG. 5A shows, in the U2OS stable hEF1α cell line, dCas9-SALL1-SUDS3 demonstrated greater gene repression against BRCA1, PSMD7, SEL1L, and ST3GAL4. As FIG. 5B shows, in the Jurkat stable hEF1α cell line, dCas9-SALL1-SUDS3 also demonstrated greater gene repression against BRCA1, PSMD7, SEL1L, and ST3GAL4. As FIG. 5C shows, in the USOS stable hEF1α cell line, dCas9-SALL1-SUDS3 demonstrated greater or similar gene repression against RAB11A, PPB, and SEL1L. As FIG. 6A shows, in the K562 stable hEF1α cell line, dCas9-SALL1-SUDS3 also demonstrated greater gene repression against BRCA1, PSMD7, SEL1L, and ST3GAL4. As FIG. 6B shows, in the A375 stable hEF1α cell line, dCas9-SALL1-SUDS3 demonstrated greater or similar gene repression against BRCA1, PSMD7, SEL1L, and ST3GAL4.

### Example 4: dCas9-KRAB Versus dCas9-SALL1-SUDS3 Over Course of 6 Days

U2OS cell lines stably expressing dCas9-SALL1-SUDS3 or dCas9-KRAB under the control of the hEF1α promoter were transfected with the pools of three synthetic sgRNAs targeting each of the following genes: BRCA1, CD46, HBP1, and SEL1L. Repression was measured over six days with samples harvested every 24 hours post-transfection. Total RNA was isolated, and gene expression was assessed via RT-qPCR. The relative expression of each gene was calculated with the ΔΔCq method using GAPDH as the housekeeping gene and normalized to a non-targeting control.

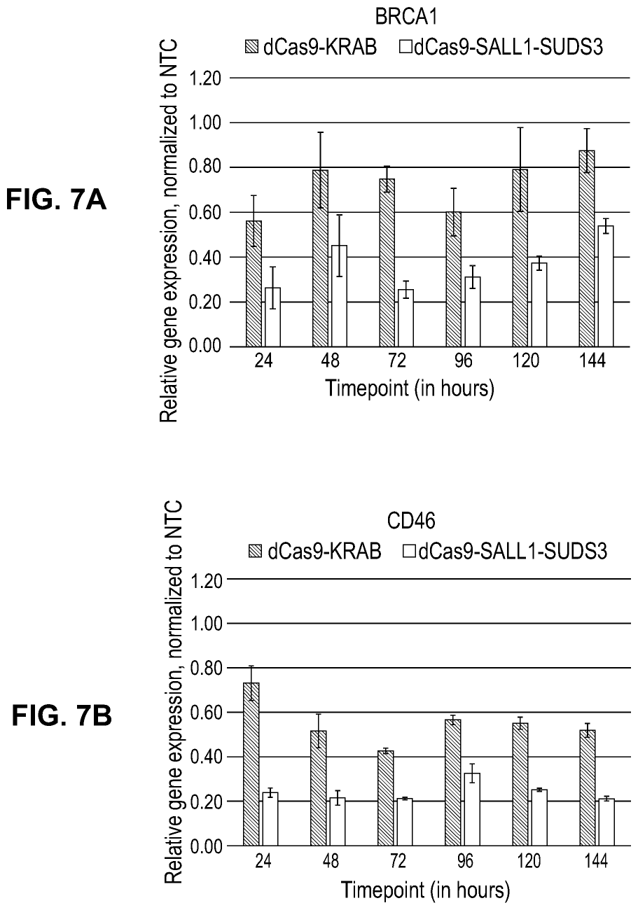

FIG. 7A shows that dCas9-SALL1-SUDS3 caused greater repression than dCas9-KRAB did against BRCA1 at all timepoints. FIG. 7B shows that dCas9-SALL1-SUDS3 caused greater repression than dCas9-KRAB did against CD46 at all timepoints. FIG. 7C shows that dCas9-SALL1-SUDS3 caused greater repression than dCas9-KRAB did against HBP1 at all timepoints. FIG. 7D shows that dCas9-SALL1-SUDS3 caused greater repression than dCas9-KRAB did against SEL1L at all timepoints. Note that in each example there was a more rapid onset of the repression mediated by dCas9-SALL1-SUDS3 than that mediated by dCas9-KRAB, and that the repression mediated by dCas9-SALL1-SUDS3 persisted at close to maximal levels for longer than the repression mediated by dCas9-KRAB.

### Example 5: Pooling sgRNAs

WTC-11 hiPSCs stably expressing dCas9-SALL1-SUDS3, and U2OS cells stably expressing dCas9-SALL1-SUDS3 were nucleofected or transfected with individual or a pool of three synthetic sgRNAs targeting PPIB (3 μM), SEL1L (3 μM), RAB11A (3 μM)-3 μM of each sgRNA electroporated, BRCA1 (25 nM), PSDM7 (25 nM), SEL1L (25 nM), and ST3GAL4 (25 nM) delivered via lipid transfection. Cells were harvested 72 hours later. The total RNA was isolated and relative gene expression was measured using RT-qPCR. Relative gene expression was calculated with the ΔΔCq method using GAPDH as the housekeeping gene and normalized to a non-targeted control.

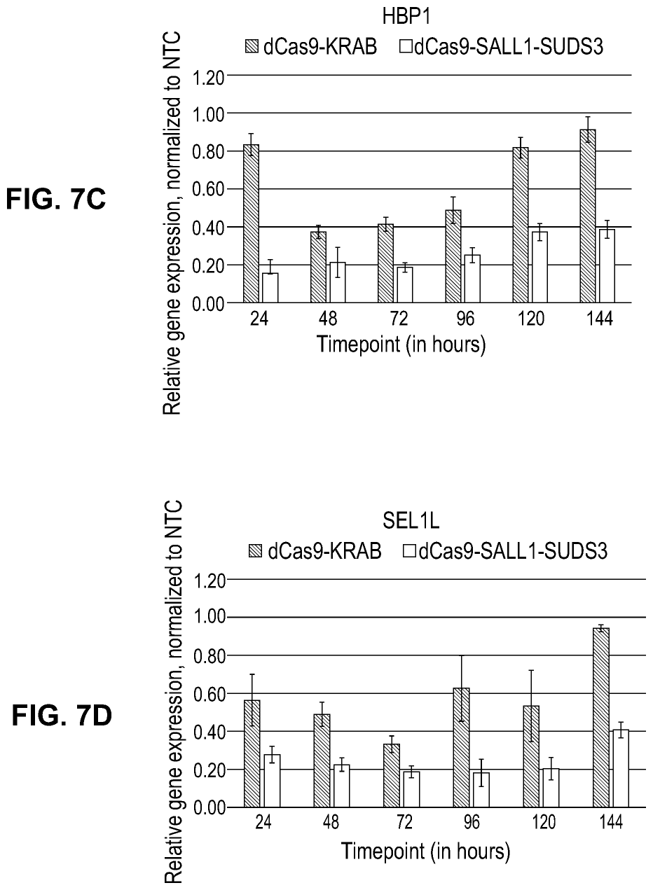

As FIG. 8A shows, in the WTC-11 hiPSCs, the pooling was comparable to or better than the use of each individual sgRNA. Similarly, as FIG. 8B shows, in the US2OS hEF1α dCas9-SALL1-SUDS3, the pooling was comparable to or better than the use of each individual sgRNA.

### Example 6: Multiplexing of gRNAs for Simultaneous Repression of Multiple Genes

hiPSCs stably expressing dCas9-SALL1-SUDS3 were nucleofected with individual sgRNAs and pools of up to 6 sgRNAs targeting unique genes. Cells were harvested 72 hours later. The total RNA was isolated and the relative gene expression was measured using RT-qPCR. The relative gene expression was calculated with the ΔΔCq method using GAPDH as the housekeeping gene and normalized to a non-targeted control

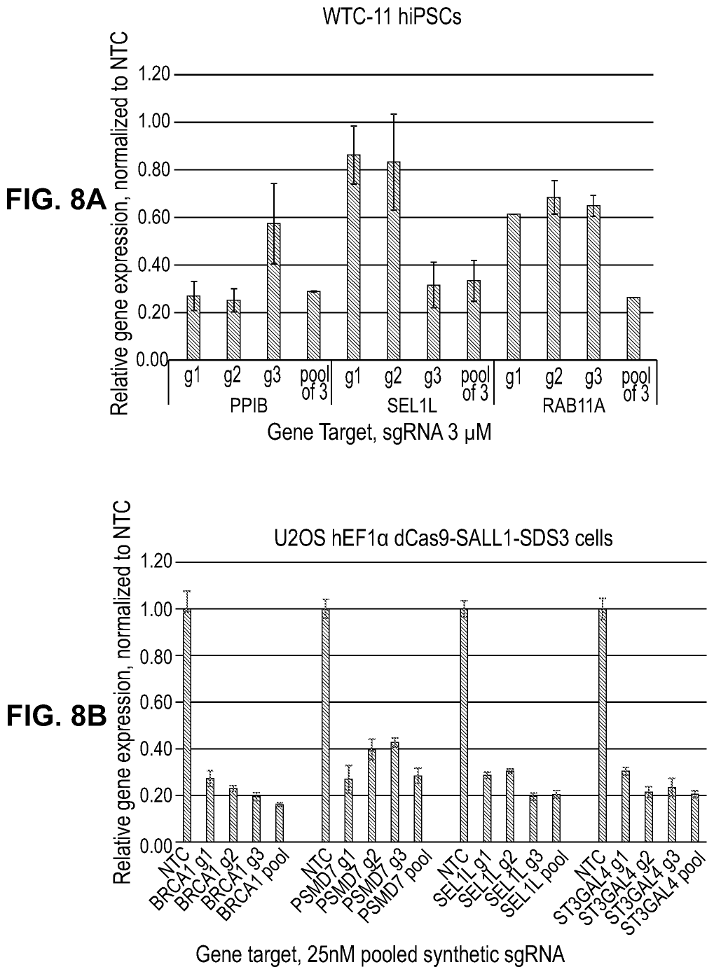

As FIG. 9 shows, when up to six genes were targeted for simultaneous repression in human iPS cells, the levels of target gene repression was comparable to when only one of the genes was targeted.

### Example 7: Fusion to N-Terminal Amino Acid and to C-Terminal Amino Acid of Cas Protein

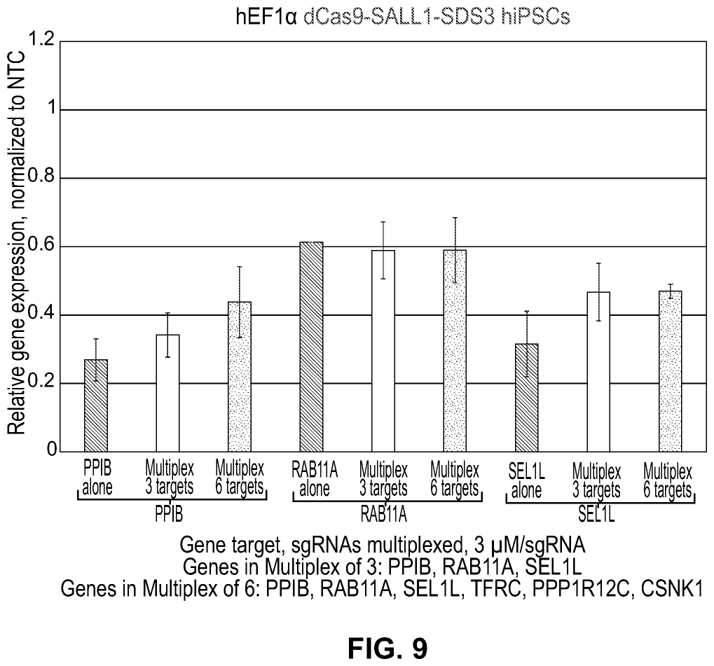

The structures of three Cas fusion proteins are represented at the bottom of FIG. 10: dCas9-KRAB; dCas9-SALL1-SUDS3, and SUDS3-SALL1-dCas9. The Cas fusion proteins were expressed under the control of the human EF1α promoter.

U2OS Ubi[G76V]-EGFP cell lines were generated that stably expressed various bipartite dCas9 fusion proteins based, along with a cell line stably expressing dCas9-KRAB. Cells were transfected with 25 nM synthetic sgRNAs targeting genes known to be critical to proteasome function, as well as non-targeting controls. The fluorescence of each transfection condition was determined at 72 hours post-transfection with an EnVision® plate reader and values were normalized to that those of the untreated cell line.

The U2OS cell line stably expressing a mutant Ubiquitin fused to enhanced green fluorescent protein (Ubi[G76V]-EGFP). In untreated cells, the expressed ubiquitin EGFP protein is constitutively degraded, leaving only background fluorescence, whereas cells with inhibited proteasome function display an accumulation of EGFP. Repression of target genes therefore results in increased fluorescence.

As FIG. 10 shows, the Cas fusion proteins containing dCas9, SUDS3, and SALL1 showed substantially more repression than dCas9-KRAB regardless of whether the fusion occurred at the N-terminal amino acid or the C-terminal amino acid of the Cas protein. (A higher mean GFP expression correlates to greater repression.)

### Example 8: Plasmid: Plasmid Co-Transfection in A375 & U2OS Cells

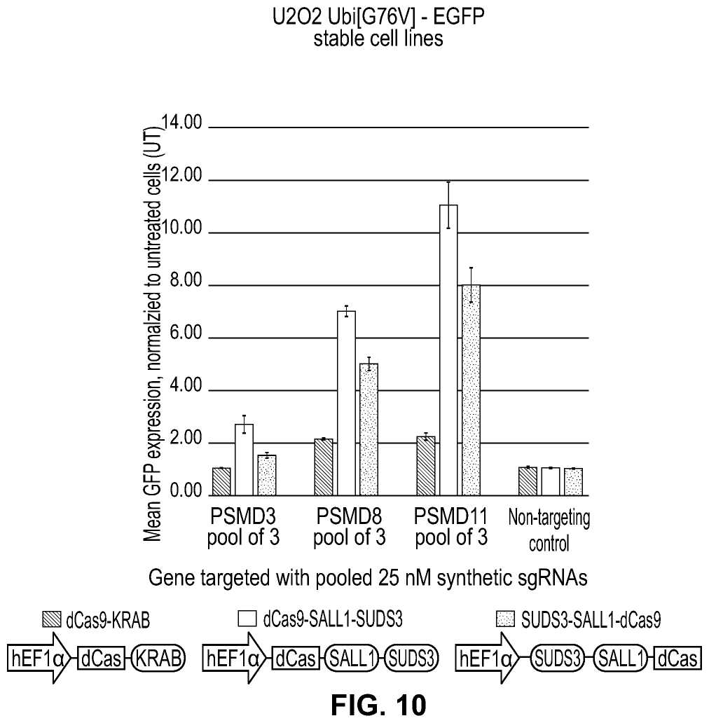

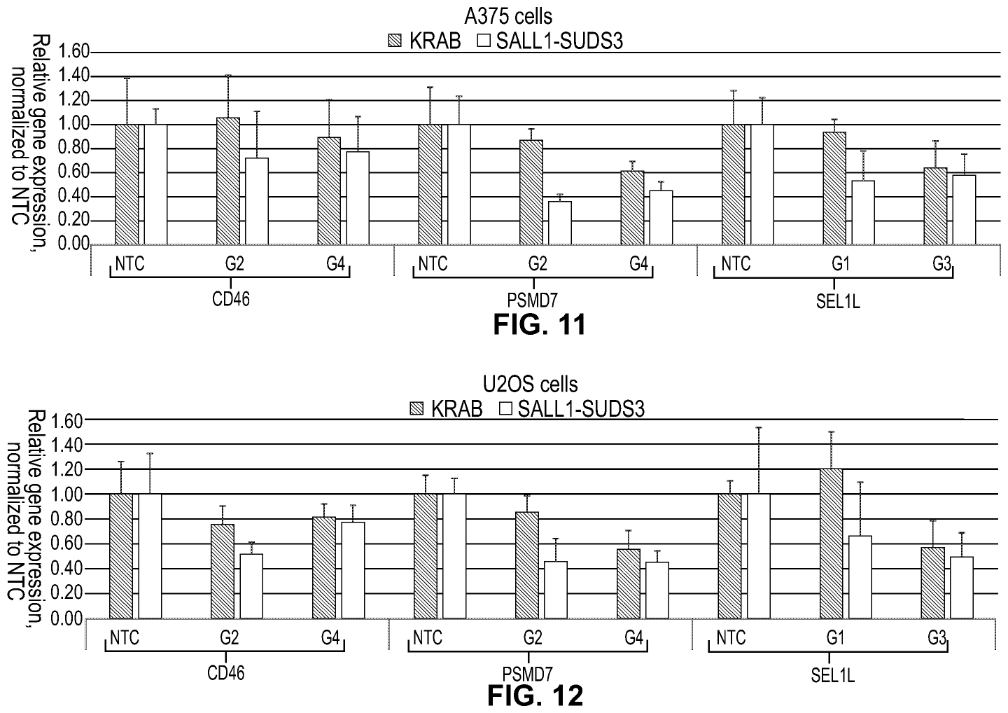

Plasmid repressors: (1) hEF1α-dCas9 KRAB; or (2) dCas9-SALL1-SUDS3 were co-transfected with guides (total=100 ng) using 0.6 μL/well of DharmaFECT® kb. FIG. 11 shows the results when measuring gene expression by RT-qPCR at three days post-plasmid co-transfection of repressor and gene targets in A375 cells. FIG. 12 shows the results when measuring gene expression by RT-qPCR at three days post-plasmid co-transfection of repressor and gene targets in U2OS cells. Both figures consistently show greater repression in systems that contained the plasmid for dCas9-SALL1-SUDS3.

### Example 9: Additional Repressors

U2OS Ubi[G76V]-EGFP cell lines were generated that stably expressed various bipartite dCas9 fusion proteins based, along with a cell line stably expressing dCas9-KRAB. Cells were transfected with 25 nM synthetic sgRNAs targeting genes known to be critical to proteasome function, as well with non-targeting controls. The fluorescence of each transfection condition was determined at 72 hours post-transfection with an Envision® plate reader. The values were normalized to those of the untreated cell line.

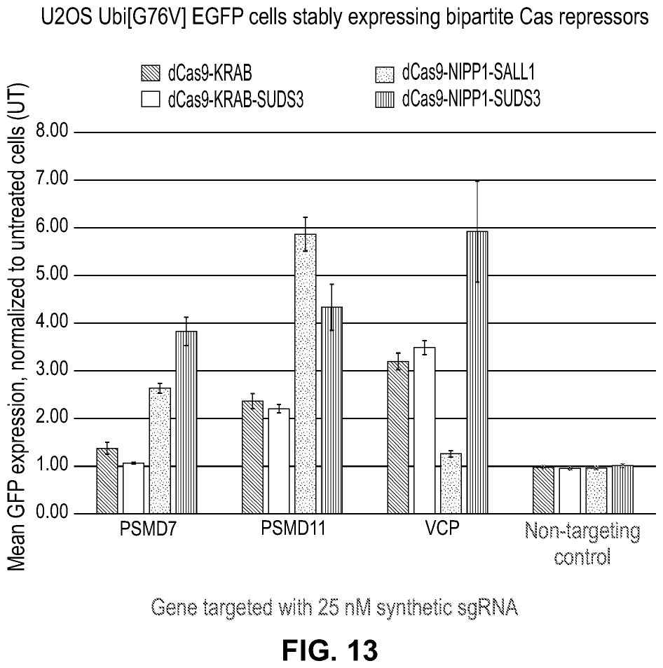

As FIG. 13 shows, a Cas fusion protein containing: (i) dCas9; (ii) either SUDS3 or SALL1; and (iii) KRAB or NIPP1 shows greater than or comparable repression as dCas9. (A taller bar indicates greater repression. The system was designed in the same manner as the system in example 8.)

### Example 10: Type V Cas Protein-SALL1-SUDS3 Fusion Constructs

A deactivated MAD7 (an engineered Cas12a protein)-SALL1-SUDS3 fusion construct was cloned (dMAD7-SALL1-SUDS3), and U2OS cells were generated that stably expressed it under control of the minimal CMV (mCMV) promoter. A deactivated CasPhi8 (a Cas12J protein)-SALL1-SUDS3 fusion construct was cloned (dCAsPhi8-SALL1-SUDS3), and U2OS cells were generated that stably expressed it under control of the mCMV promoter. These cells, along with U2OS cells stably expressing dMAD7 or dCasPhi8, were lipid transfected with synthetic guides designed for the respective Cas proteins, in each case delivered at 25 nM. Transcriptional repression was assessed 48 hours post-transfection.

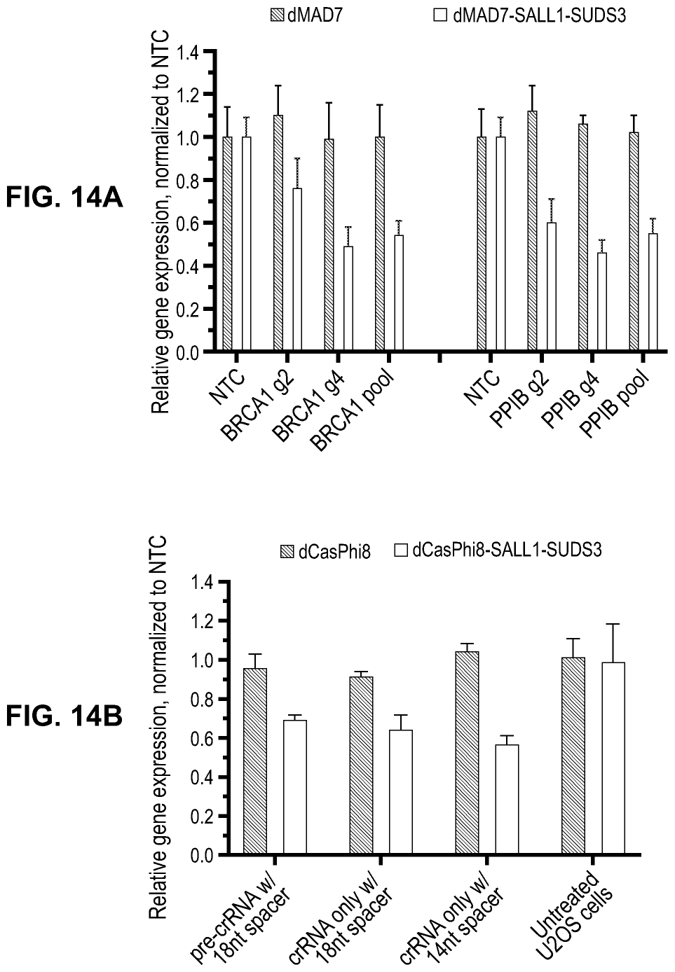

FIG. 14A shows CRISPRi induced transcriptional repression in U2OS cells stably expressing either dMAD7 or dMAD7-SALL1-SUDS3 for two individual synthetic guide RNAs against each of BRCA1 and PPIB, as well as for a pool of synthetic guide RNA, and an NTC. The figure shows significantly greater repression effected by dMAD7-SALL1-SUDS3 as compared to dMAD7.

FIG. 14B shows CRISPRi induced transcriptional repression in U2OS cells stably expressing either dCasPhi8 or dCasPhi8-SALL1-SUDS3 for three iterations of individual synthetic guide RNAs targeting the same site in BRCA1, and basal BRCA1 expression in untreated U2OS cells. The figure shows significantly greater repression effected by dCasPhi8-SALL1-SUDS3 as compared to dCasPhi8.

These figures demonstrate that SALL1 and SUDS3 can be fused to various Type V Cas proteins and programmed with synthetic guide RNA to effect significant target gene repression.

### Example 11: Simultaneous Editing and Repression with Active Cas9 Fusion Proteins

U2OS cells stably expressing SUDS3-SALL1-WtCas9 under the control of the hEF1α promoter were transfected with 25 nM pools of guide RNAs designed for both CRISPRi and CRISPR editing. Guides designed for CRISPRi contained a truncated 14-mer targeting region. Guides designed for CRISPR editing contained the full 20-mer targeting region. Cells were harvested 72 hours later post-transfection. The total RNA was isolated and the relative gene expression was measured using RT-qPCR. The relative gene expression was calculated with the ΔΔCq method using GAPDH as the housekeeping gene and normalized to a non-targeted control. Genomic DNA was isolated, target regions were amplified using PCR and Sanger sequenced, and indel formation was analyzed using TIDE.

FIG. 15B shows MRE11A can be repressed while LBR is simultaneously edited. FIG. 15C shows MRE11A can be repressed while PPIB is simultaneously edited. FIG. 15D shows SEL1L can be repressed while LBR is simultaneously edited. FIG. 15E shows SEL1L can be repressed while PPIB is simultaneously edited.

### Example 12: Comparison of Single Repressor Domains as dCas9-Fusion Protein

U2OS Ubi[G76V]-EGFP cell lines were generated that stably expressed various single repressor dCas9 fusion proteins (BCL6, CbpA, H-NS, MBD3, NIPP1, SALL1, and SUDS3), along with a cell line stably expressing dCas9-KRAB, all under the control of the human EF1α promoter. Cells were transfected with 25 nM synthetic sgRNAs targeting genes known to be critical to proteasome function, as well as non-targeting controls.

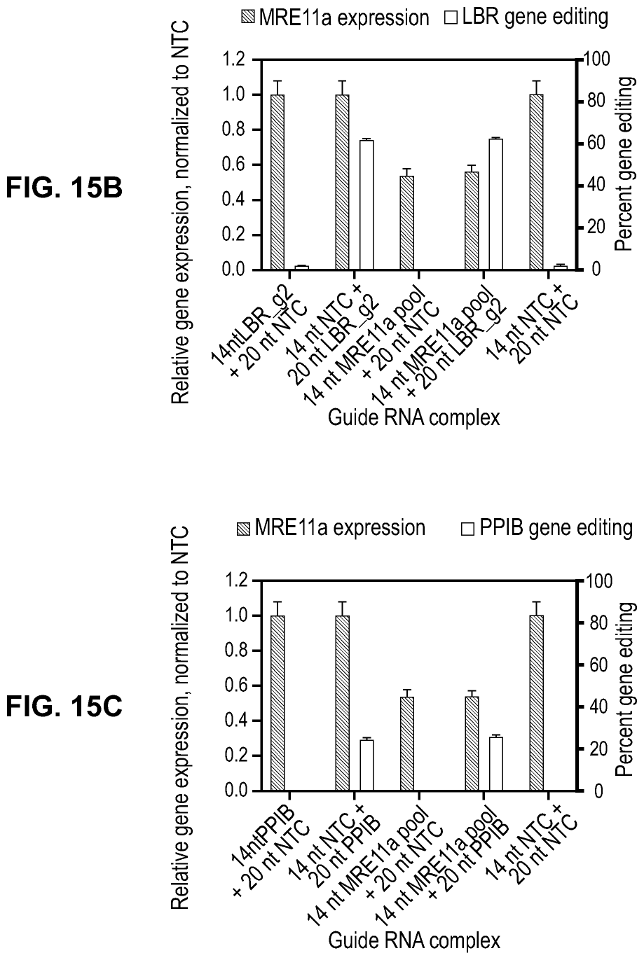

The fluorescence of each transfection condition was determined at 72 hours post-transfection, with an EnVision® plate reader and values were normalized to those of the untreated cell line. The U2OS cell line stably expressed a mutant Ubiquitin fused to enhanced green fluorescent protein (Ubi[G76V]-EGFP). In untreated cells, the expressed ubiquitin EGFP is constitutively degraded, leaving only background fluorescence, whereas cells with inhibited proteasome function display an accumulation of EGFP. Repression of target genes therefore results in increased fluorescence. As FIG. 16 shows, the dCas fusion proteins containing NIPP1, SALL1, and SUDS3, showed substantially more repression than dCas9-KRAB. (A higher mean GFP expression correlates to greater repression.)

### Example 13: Comparison of dCas9-SUDS3 Repressor to dCa9-KRAB and dCas9-KRAB-MeCP2 Systems

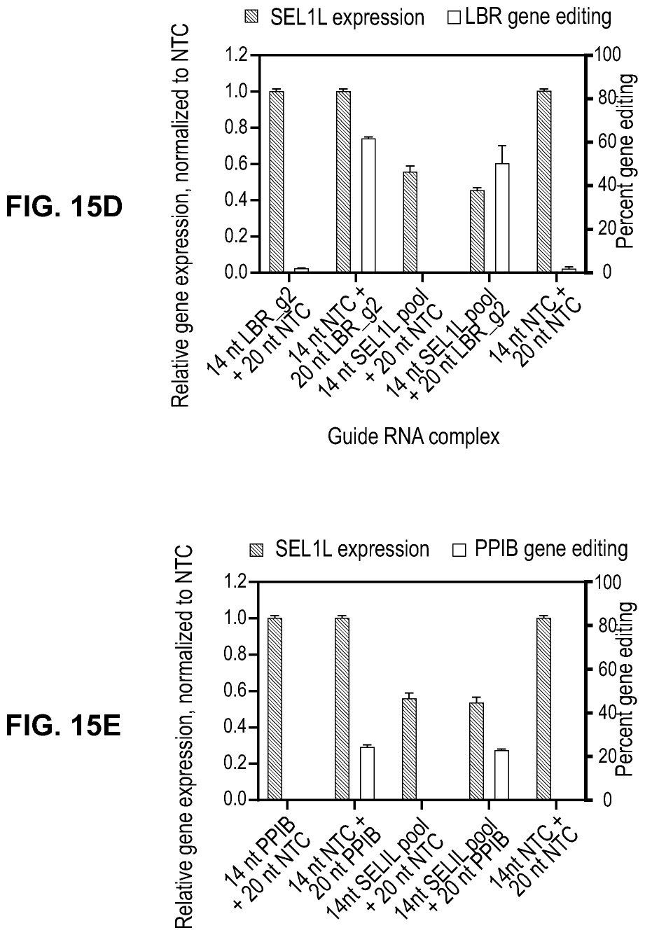

U2OS Ubi[G76V]-EGFP cell lines were generated that stably expressed either dCas9-KRAB, dCas9-KRAB MeCP2, or dCas9-SUDS3 under the control of the human EF1α promoter. Cells were transfected with 25 nM synthetic sgRNAs targeting genes known to be critical to proteasome function, as well as non-targeting controls. Cells were harvested 72 hours post-transfection, total RNA was isolated, and expression of the target genes was assessed via RT-qPCR. Relative expression was calculated with the ΔΔCq method using GAPDH as the housekeeping gene and normalized to a non-targeting control. FIG. 17 shows that for each gene target dCas9-SUDS3 effected substantially more transcriptional repression than either dCas9-KRAB or dCas9-KRAB-MeCP2.

### Example 14: Proteasome Functional Reporter Assay and Transcriptional Repression

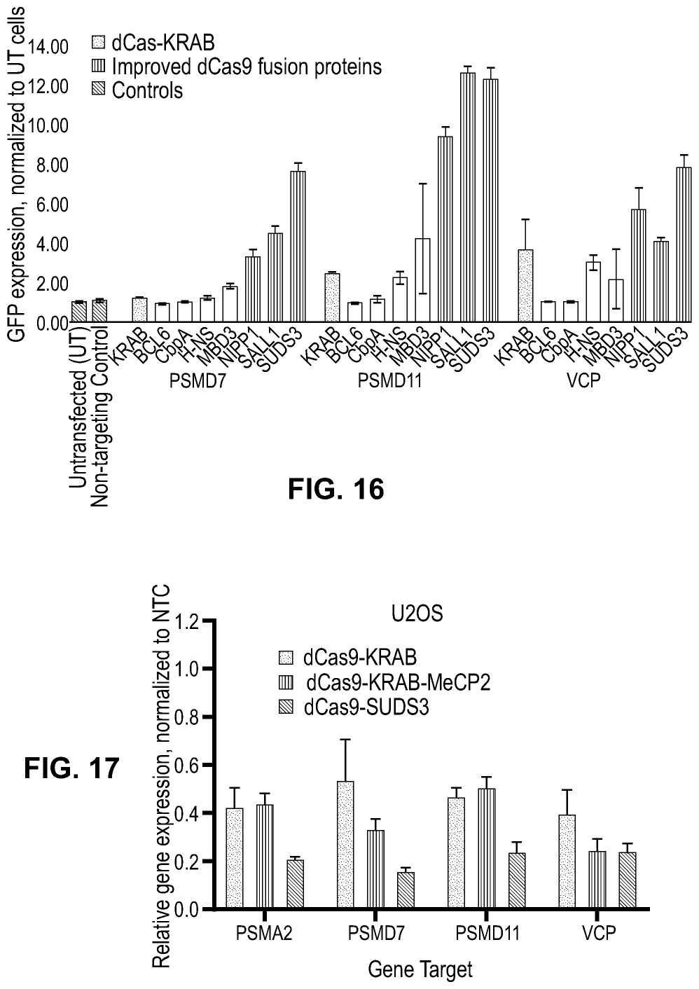

U2OS Ubi[G76V]-EGFP cell lines were generated that stably expressed either dCas9-KRAB or dCas9-SALL1-SUDS3 under the control of the human EF1α promoter. Cells were transfected with 25 nM synthetic sgRNAs targeting genes known to be critical to proteasome function, as well as non-targeting controls. The fluorescence of each transfection condition was determined at 72 hours post-transfection, with an EnVision® plate reader and values were normalized to those of the untreated cell line. The U2OS cell line stably expressed a mutant Ubiquitin fused to enhanced green fluorescent protein (Ubi[G76V]-EGFP). In untreated cells, the expressed ubiquitin EGFP is constitutively degraded, leaving only background fluorescence, whereas cells with inhibited proteasome function display an accumulation of EGFP. Repression of target genes therefore results in increased fluorescence. Total RNA was also isolated and expression of the target genes was assessed via RT-qPCR. Relative expression was calculated with the ΔΔCq method using GAPDH as the housekeeping gene and normalized to a non-targeting control. FIG. 18A shows dCas9-SALL1-SUDS3 effected significantly more phenotypic knockdown than dCas9-KRAB. (A higher mean GFP expression correlates to greater repression.) FIG. 18B shows that the more pronounced phenotype observed with dCas9-SALL1-SUDS3 correlated with increased transcriptional repression of the targeted proteasome genes.

### Example 15: Lentiviral Delivery

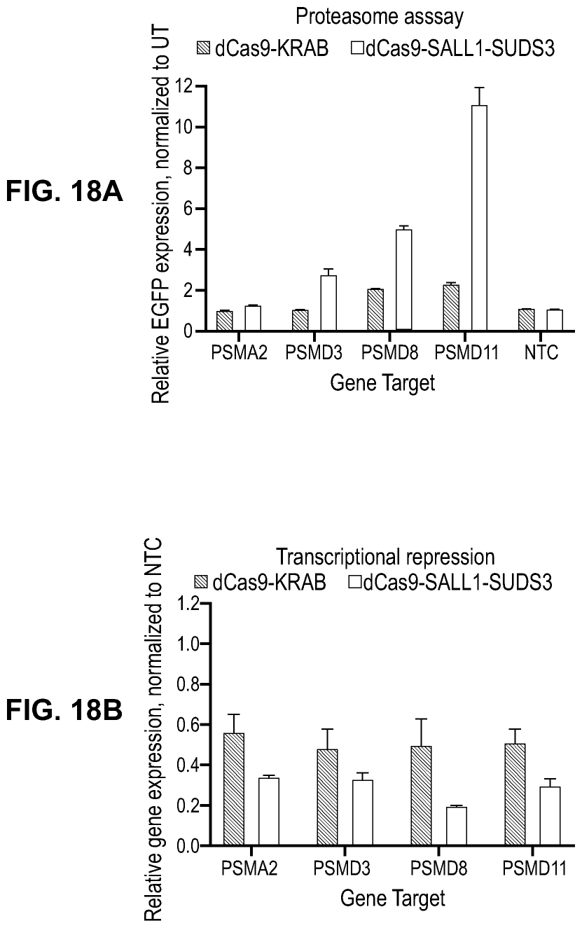

FIG. 19A shows the transcriptional repression of PPIB and SEL1L in U2OS cells stably expressing either dCas9-SALL1-SUDS3 and a guide RNA from a single lentiviral vector or from two separate vectors. FIG. 19B shows the transcriptional repression of PPIB and SEL1L in HCT 116 cells stably expressing either dCas9-SALL1-SUDS3 and a guide RNA from a single lentiviral vector or from two separate vectors.

Lentiviral vectors were used to generate U2OS and HCT 116 cells that stably expressed dCas9-SALL1-SUDS3 under the control of the human EF1α promoter (hEF1a) or mouse CMV promoter (mCMV) respectively. These cells were subsequently transduced with lentiviral particles containing vectors that expressed individual guide RNAs from the human U6 promoter and targeted PPIB, SEL1L, or contained a non-targeting control sequence. Parental U2OS and HCT 116 cells were transduced with lentiviral particles containing a single vector that expressed dCas9-SALL1-SUDS3 under the control of the hEF1α (U2OS) or mCMV (HCT 116) promoters, and an individual guide RNA from the human U6 promoter. These single vector systems also targeted PPIB or SEL1L, or contained a non-targeting control sequence. Twenty-four hours post-transduction, media containing 2.5 μg/mL puromycin was added to enrich for transduced cells. Cells were cultured in this media for 7 days and passaged every 3 to 4 days. Eight days post-transduction cells were harvested, total RNA was isolated, and the relative expression of the target genes was determined by RT-qPCR. Relative gene expression was calculated with the ΔΔCq method using GAPDH as the housekeeping gene and normalized to the non-targeting control.

FIG. 19A shows that either a single lentiviral or dual lentiviral vector system can be used to express dCas9-SALL1-SUDS3 and a guide RNA to robustly repress a target gene in U2OS cells. FIG. 19B shows that either a single lentiviral or dual lentiviral system can be used to express dCas9-SALL1-SUDS3 and a guide RNA to robustly repress a target gene in HCT 116 cells.

### Example 16: Plasmid sgRNAs vs. Synthetic sgRNAs

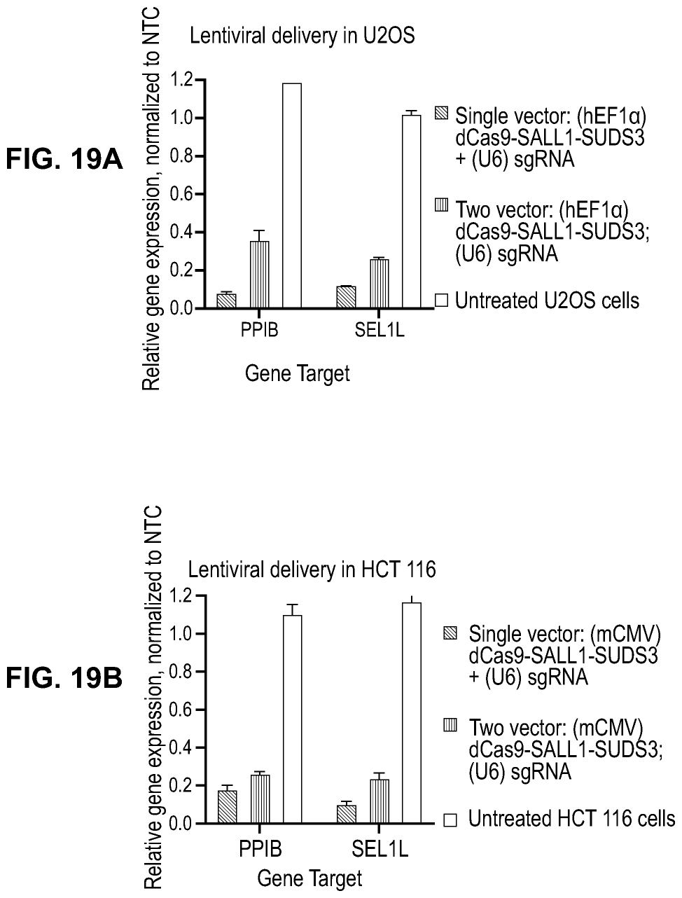

U2OS and A375 cells stably expressing dCas9-SALL1-SUDS3 under the control of the hEF1α promoter were transfected with individual, matched 25 nM synthetic sgRNAs or 100 ng of plasmid sgRNA targeting BRCA1, PSMD7, SEL1L, and ST3GAL4. Cells were harvested 72 hours post-transfection, total RNA was isolated, and the relative gene expression of each target genes was assessed using RT-qPCR. Relative gene expression was calculated with the ΔΔCq method using GAPDH as the housekeeping gene and normalized to a non-targeted control. FIG. 20A demonstrates that dCas9-SALL1-SUDS3 mediates substantially greater target gene expression when delivered with synthetic sgRNAs than when delivered with plasmid sgRNAs in U2OS cells. FIG. 20B demonstrates that dCas9-SALL1-SUDS3 mediates substantially more target gene expression when delivered with synthetic sgRNAs than when delivered with plasmid sgRNAs in A375 cells.

### Example 17: Synthetic sgRNA vs crRNA:tracrRNA

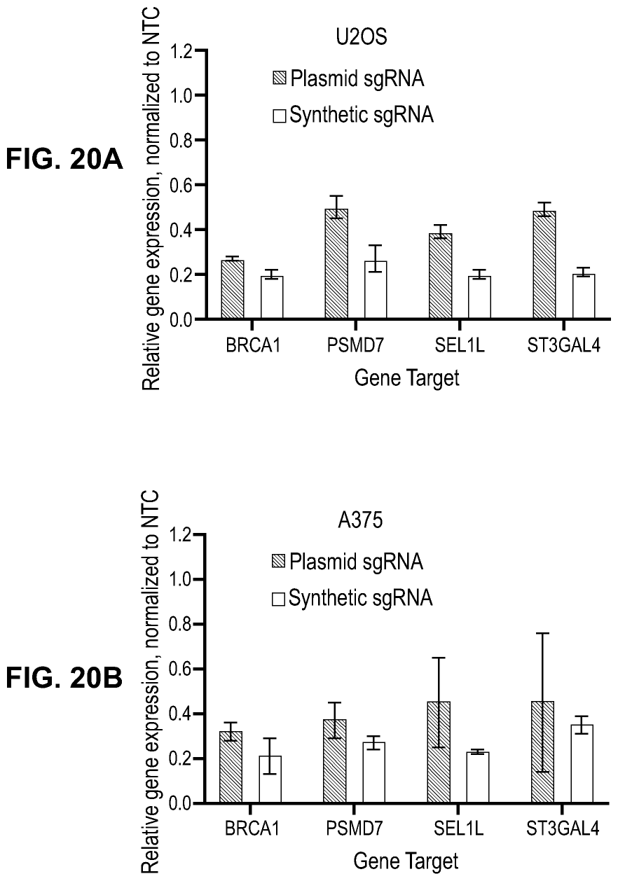

U2OS cells stably expressing dCas9-SALL1-SUDS3 under the control of the hEF1α promoter were transfected with pooled 25 nM synthetic sgRNAs or synthetic crRNA:tracrRNA complexes. Cells were harvested 72 hours post-transfection, total RNA was isolated, and the relative gene expression of each target genes was measured using RT-qPCR. Relative gene expression was calculated with the ΔΔCq method using GAPDH as the housekeeping gene and normalized to a non-targeted control. FIG. 21 demonstrates that while repression is markedly more pronounced with pooled synthetic sgRNAs, both synthetic sgRNA and synthetic crRNA:tracrRNA complexes can be delivered with dCas9-SALL1-SUDS3 to cause target gene repression.

### Example 18: 5′ Truncated Spacer

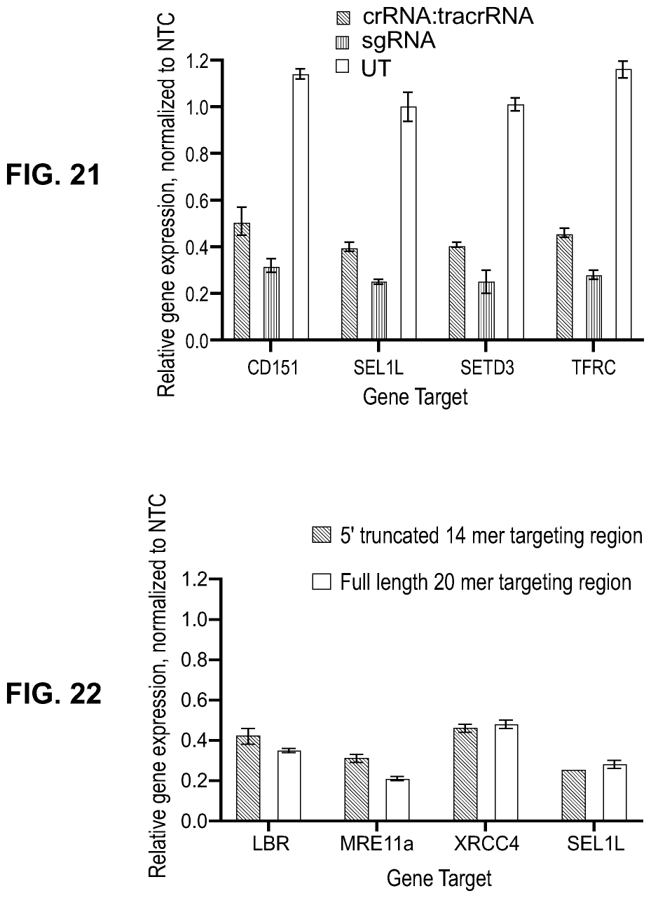

U2OS cells stably expressing dCas9-SALL1-SUDS3 under the control of the hEF1α promoter were transfected with 25 nM pools of guide RNAs containing either truncated 14-mer targeting regions or full length 20-mer targeting regions. Cells were harvested 72 hours post-transfection. Total RNA was isolated and the relative gene expression of the target genes was measured using RT-qPCR. Relative gene expression was calculated with the ΔΔCq method using GAPDH as the housekeeping gene and normalized to a non-targeted control. FIG. 22 shows that the targeting region of a guide RNA can be shortened at the 5′ end by at least 6-mer and still effect transcriptional repression when delivered with dCas9-SALL1-SUDS3.

### Example 19: LNA Modified sgRNAs

U2OS Ubi[G76V]-EGFP cells stably expressing dCas9-SALL1-SUDS3 under the control of the human EF1α promoter were transfected with 25 nM synthetic sgRNAs targeting two genes known to be critical to proteasome function, as well as non-targeting controls. The guides contained various combinations of 2′-O-methyl and phosphorothioate linkages and locked nucleic acids at the ends of the sgRNA molecule, and in the 20-mer targeting region, position 1 to position 20 from the 5′ end. The fluorescence of each transfection condition was determined 144 hours post-transfection with an EnVision® plate reader and values were normalized to those of the untreated cell line. The U2OS cell line stably expressed a mutant Ubiquitin fused to enhanced green fluorescent protein (Ubi[G76V]-EGFP). In untreated cells, the expressed ubiquitin EGFP is constitutively degraded, leaving only background fluorescence, whereas cells with inhibited proteasome function display an accumulation of EGFP. Repression of target genes therefore results in increased fluorescence.

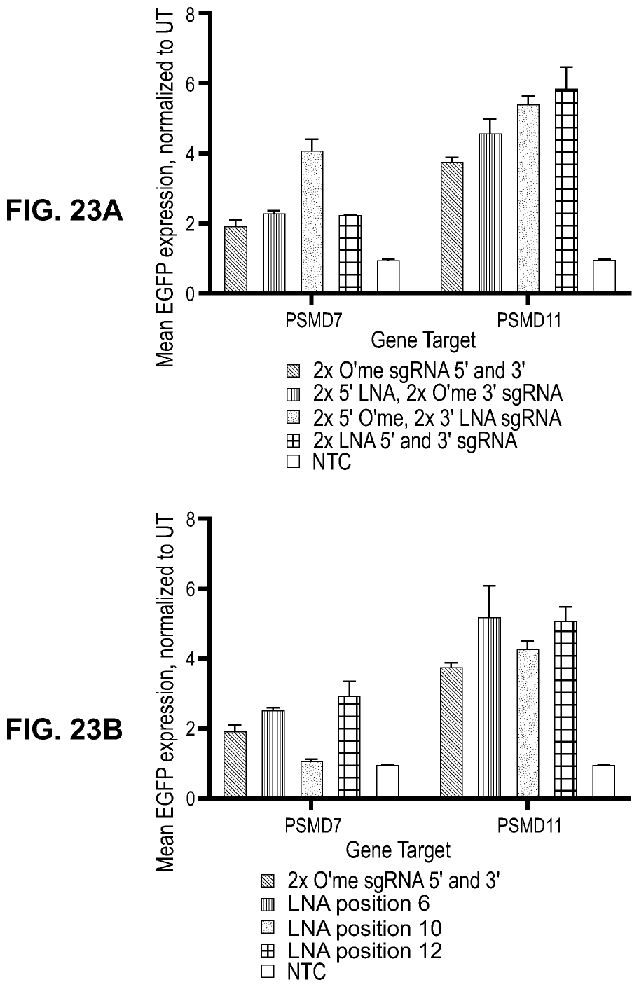

FIG. 23A shows the effects on dCas9-SALL1-SUDS3 mediated functional knockdown of various chemical end modifications to the sgRNA molecule. The incorporation of locked nucleic acids at the 5′ and 3′ end of the sgRNA molecule can be used to stabilize the gRNA. The incorporation of two locked nucleic acid at the 3′ end of the sgRNAs targeting PSMD7 and PSMD11 further improves target gene repression. (A higher mean GFP expression correlates to greater repression.) FIG. 23B shows the impact of the incorporation of locked nucleic acid positions into the sgRNA targeting region on dCas9-SALL1-SUDS3 mediated functional knockdown. Locked nucleic acids can be incorporated at some positions of the sgRNA targeting region to improve target gene repression.

### Example 20: RNA-Repressor Complex Recruitment

U2OS cells stably expressing dCas9 and SALL1-SUDS3 fused to the MS2 Coat protein ligand (MCP-SALL1-SUDS3), each under the control of the human EF1α promoter, were generated through sequential transduction of the respective lentiviral expression vector. The cells were then transfected with 25 nM synthetic crRNA:tracrRNA complexes targeting BRCA1, CD151, and SETD3, along with NTCs. Several tracrRNA designs containing different MS2 ligand binding moiety sequences and positions were tested against each gene target and compared to complexes containing a tracrRNA without an MS2 ligand binding moiety, labeled crRNA:tracrRNA w/out MS2. Cells were harvested 72 hours post-transfection, total RNA was isolated, and the relative gene expression of each target genes was measured using RT-qPCR. Relative gene expression was calculated with the ΔΔCq method using GAPDH as the housekeeping gene and normalized to a non-targeting control.

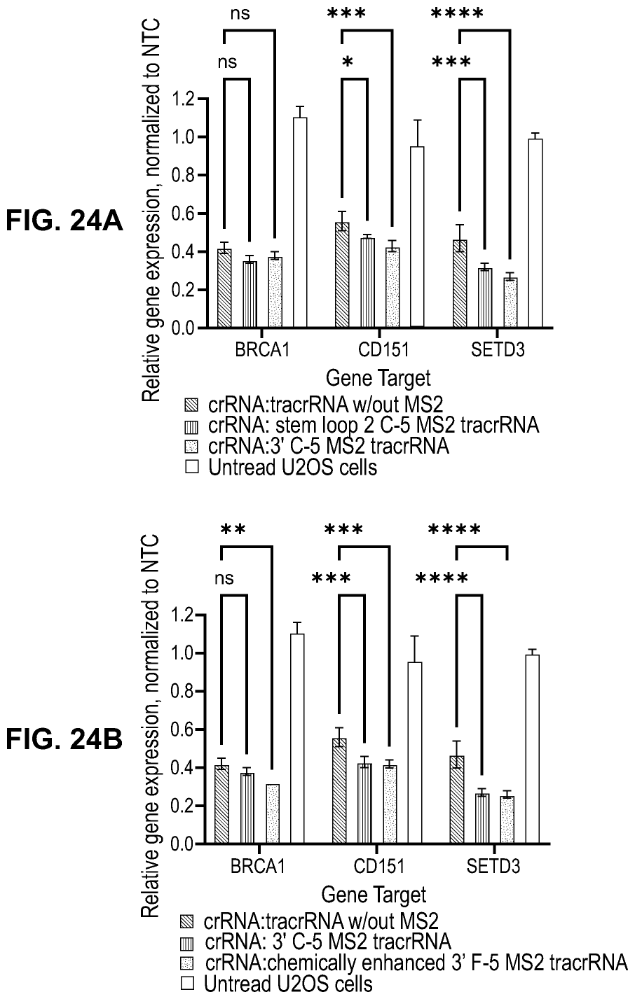

FIG. 24A demonstrates that MCP-SALL1-SUDS3 can be recruited to dCas9 through the C-5 MS2 sequence positioned at the either sgRNA stem loop 2 or at the 3′ terminus of the tracrRNA molecule. The recruitment of MCP-SALL1-SUDS3 can enhance the repressive effect of dCas9 binding, represented here as crRNA:tracrRNA w/out MS2. FIG. 24B shows that MCP-SALL1-SUDS3 can be recruited to dCas9 through both the C-5 MS2 sequence and the F-5 MS2 sequence containing a 2dAP chemical mod to significantly improve the repressive effect of dCas9 binding.

### Example 21: Knockdown in T-Cells

Primary human CD4+ T cells were nucleofected with dCas9-SALL1-SUDS3 mRNA and pooled synthetic sgRNA via a Lonza 96-well Shuttle system. 24 and 72 hours post-nucleofection, functional knockdown of CXCR3 was assessed as a percent of cells expressing the target gene by FACS analysis. Cells were stained for CD4 as a positive expression control using an Alexa Fluor 488 conjugated antibody and compared to CXCR3 using APC conjugated primary antibodies. Total RNA was isolated at each timepoint and mRNA expression of CXCR3 was assessed via RT-qPCR. The relative expression of CXCR3 was calculated with the ΔΔCq method using GAPDH as the housekeeping gene and normalized to a non-targeting control.

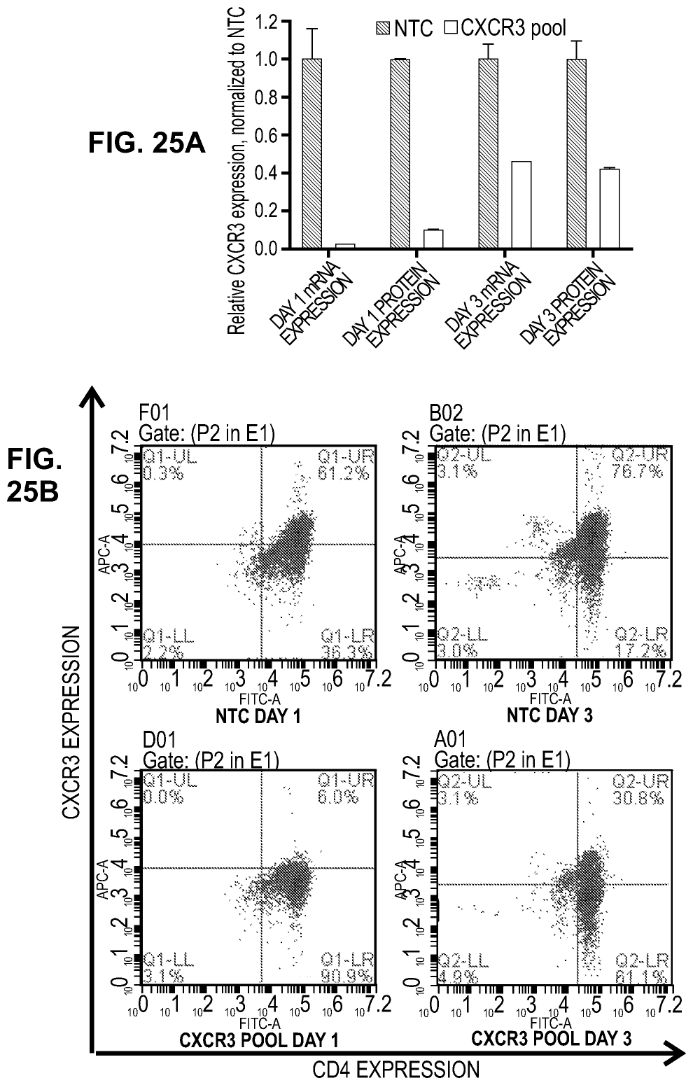

FIG. 25A is a graph that shows the transcriptional repression and protein level knockdown of CXCR3 in primary human CD4+ T cells nucleofected with dCas9-SALL1-SUDS3 and either a synthetic non-targeting control or a pool of 3 guides targeting the gene of interest 1 and 3 days post-nucleofection.

FIG. 25B shows that the onset of knockdown with dCas9-SALL1-SUDS3 was rapid and persisted for several days in this clinically relevant primary cell type, comparing protein expression in the non-transfected control system on day 1, protein expression in the non-transfected control system on day 3, protein expression in the CXCR3 pool system on day 1 and protein expression in the CXCR3 pool system on day 3.

TABLE 2
synthetic sgRNAs (Sp Cas9)
Target region is bolded, chemical modifications are italicized.

| Target | Guide Name | SEQUENCE |
|---|---|---|
| NTC | NTC | (mG)*(mU)*AACGCGAACUACGCGGGUGUUUUAGAGCUAGAAAUAG |
| | sgRNA | CAAGUUAAAAUAAGGCUAGUCCGUUAUCAACUUGAAAAAGUGGCA |
| | | CCGAGUCGGUGCU(mU)*(mU)*U (SEQ ID: 43) |
| BRCA1 | BRCA1_g1 | (mC)*(mU)*CGCUGAGACUUCCUGGACGUUUUAGAGCUAGAAAUAG |
| | | CAAGUUAAAAUAAGGCUAGUCCGUUAUCAACUUGAAAAAGUGGCA |
| | | CCGAGUCGGUGCU(mU)*(mU)*U (SEQ ID: 44) |
| | BRCA1_g2 | (mU)*(mG)*AAGGCCUCCUGAGCGCAGGUUUUAGAGCUAGAAAUAG |
| | | CAAGUUAAAAUAAGGCUAGUCCGUUAUCAACUUGAAAAAGUGGCA |
| | | CCGAGUCGGUGCU(mU)*(mU)*U (SEQ ID: 45) |
| | BRCA1_g3 | (mC)*(mC)*ACAGCCUGUCCCCCGUCCGUUUUAGAGCUAGAAAUAG |
| | | CAAGUUAAAAUAAGGCUAGUCCGUUAUCAACUUGAAAAAGUGGCA |
| | | CCGAGUCGGUGCU(mU)*(mU)*U (SEQ ID: 46) |
| CD46 | CD46_g1 | (mU)*(mC)*CCUUCUGGGUCCAGAUAUGUUUUAGAGCUAGAAAUAG |
| | | CAAGUUAAAAUAAGGCUAGUCCGUUAUCAACUUGAAAAAGUGGCA |
| | | CCGAGUCGGUGCU(mU)*(mU)*U (SEQ ID: 47) |
| | CD46_g2 | (mG)*(mG)*AUUGUUGCGUCCCAUAUCGUUUUAGAGCUAGAAAUAG |
| | | CAAGUUAAAAUAAGGCUAGUCCGUUAUCAACUUGAAAAAGUGGCA |
| | | CCGAGUCGGUGCU(mU)*(mU)*U (SEQ ID: 48) |
| | CD46_g3 | (mG)*(mA)*CUAGAGCUCUCCUCAGUCGUUUUAGAGCUAGAAAUAG |
| | | CAAGUUAAAAUAAGGCUAGUCCGUUAUCAACUUGAAAAAGUGGCA |
| | | CCGAGUCGGUGCU(mU)*(mU)*U (SEQ ID: 49) |
| PPIB | PPIB_g1 | (mC)*(mG)*GAGAGGCGCAGCAUCCACGUUUUAGAGCUAGAAAUAG |
| | | CAAGUUAAAAUAAGGCUAGUCCGUUAUCAACUUGAAAAAGUGGCA |
| | | CCGAGUCGGUGCU(mU)*(mU)*U (SEQ ID: 50) |
| | PPIB_g2 | (mA)*(mG)*AGGCGCAGCAUCCACAGGGUUUUAGAGCUAGAAAUAG |
| | | CAAGUUAAAAUAAGGCUAGUCCGUUAUCAACUUGAAAAAGUGGCA |
| | | CCGAGUCGGUGCU(mU)*(mU)*U (SEQ ID: 51) |
| | PPIB_g3 | (mG)*(mG)*ACCCCGCGAUGAGGGCGGGUUUUAGAGCUAGAAAUAG |
| | | CAAGUUAAAAUAAGGCUAGUCCGUUAUCAACUUGAAAAAGUGGCA |
| | | CCGAGUCGGUGCU(mU)*(mU)*U (SEQ ID: 52) |
| PSMD7 | PSMD7_g1 | (mA)*(mA)*CUGGGCCUGAAAGGGUACGUUUUAGAGCUAGAAAUAG |
| | | CAAGUUAAAAUAAGGCUAGUCCGUUAUCAACUUGAAAAAGUGGCA |
| | | CCGAGUCGGUGCU(mU)*(mU)*U (SEQ ID: 53) |
| | PSMD7_g2 | (mG)*(mC)*GCCGCCGGCCCAGCUAUAGUUUUAGAGCUAGAAAUAG |
| | | CAAGUUAAAAUAAGGCUAGUCCGUUAUCAACUUGAAAAAGUGGCA |
| | | CCGAGUCGGUGCU(mU)*(mU)*U (SEQ ID: 54) |
| | PSMD7_g3 | (mU)*(mC)*CCUGCCACACGCAAACACGUUUUAGAGCUAGAAAUAG |
| | | CAAGUUAAAAUAAGGCUAGUCCGUUAUCAACUUGAAAAAGUGGCA |
| | | CCGAGUCGGUGCU(mU)*(mU)*U (SEQ ID: 55) |
| SEL1 | SEL1L_g1 | (mA)*(mG)*GGGGCGGAUACUGACCCGGUUUUAGAGCUAGAAAUAG |
| | | CAAGUUAAAAUAAGGCUAGUCCGUUAUCAACUUGAAAAAGUGGCA |
| | | CCGAGUCGGUGCU(mU)*(mU)*U (SEQ ID: 56) |
| | SEL1L_g2 | (mA)*(mU)*ACUGACCCGAGGACGCCGGUUUUAGAGCUAGAAAUAG |
| | | CAAGUUAAAAUAAGGCUAGUCCGUUAUCAACUUGAAAAAGUGGCA |
| | | CCGAGUCGGUGCU(mU)*(mU)*U (SEQ ID: 57) |
| | SEL1L_g3 | (mG)*(mG)*UGGUGGCUGAGUCCGUGGGUUUUAGAGCUAGAAAUAG |
| | | CAAGUUAAAAUAAGGCUAGUCCGUUAUCAACUUGAAAAAGUGGCA |
| | | CCGAGUCGGUGCU(mU)*(mU)*U (SEQ ID: 58) |
| ST3GAL4 | ST3GALA_g1 | (mC)*(mC)*GCUAGGCGCACCGACCGGGUUUUAGAGCUAGAAAUAG |
| | | CAAGUUAAAAUAAGGCUAGUCCGUUAUCAACUUGAAAAAGUGGCA |
| | | CCGAGUCGGUGCU(mU)*(mU)*U (SEQ ID: 59) |
| | ST3GALA_g2 | (mG)*(mA)*UCCGCUAGGCGCACCGACGUUUUAGAGCUAGAAAUAG |
| | | CAAGUUAAAAUAAGGCUAGUCCGUUAUCAACUUGAAAAAGUGGCA |
| | | CCGAGUCGGUGCU(mU)*(mU)*U (SEQ ID: 60) |
| | ST3GALA_g3 | (mG)*(mC)*UGGCGCGACGGCUCGACUGUUUUAGAGCUAGAAAUAG |
| | | CAAGUUAAAAUAAGGCUAGUCCGUUAUCAACUUGAAAAAGUGGCA |
| | | CCGAGUCGGUGCU(mU)*(mU)*U (SEQ ID: 61) |
| RAB11A | RAB11A_g1 | (mU)*(mG)*CGCGGCCGAGGAGCGAAAGUUUUAGAGCUAGAAAUAG |
| | | CAAGUUAAAAUAAGGCUAGUCCGUUAUCAACUUGAAAAAGUGGCA |
| | | CCGAGUCGGUGCU(mU)*(mU)*U (SEQ ID: 62) |
| | RAB11A_g2 | (mG)*(mC)*GGCCGAGGAGCGAAAGGGGUUUUAGAGCUAGAAAUAG |
| | | CAAGUUAAAAUAAGGCUAGUCCGUUAUCAACUUGAAAAAGUGGCA |
| | | CCGAGUCGGUGCU(mU)*(mU)*U (SEQ ID: 63) |
| | RAB11A_g3 | (mG)*(mG)*AGCAGCAGUGGUAUCUGUGUUUUAGAGCUAGAAAUAG |
| | | CAAGUUAAAAUAAGGCUAGUCCGUUAUCAACUUGAAAAAGUGGCA |
| | | CCGAGUCGGUGCU(mU)*(mU)*U (SEQ ID: 64) |
| HBP1 | HBP1_g1 | (mA)*(mA)*GCUUGAAAGACUUGGUAAGUUUUAGAGCUAGAAAUAG |
| | | CAAGUUAAAAUAAGGCUAGUCCGUUAUCAACUUGAAAAAGUGGCA |
| | | CCGAGUCGGUGCU(mU)*(mU)*U (SEQ ID: 65) |
| | HBP1_g2 | (mU)*(mU)*GAGGAGUAAGAGCUGCCGGUUUUAGAGCUAGAAAUAG |
| | | CAAGUUAAAAUAAGGCUAGUCCGUUAUCAACUUGAAAAAGUGGCA |
| | | CCGAGUCGGUGCU(mU)*(mU)*U (SEQ ID: 66) |
| | HBP1_g3 | (mU)*(mG)*GCGACGGGUUUGGUAAGUGUUUUAGAGCUAGAAAUAG |
| | | CAAGUUAAAAUAAGGCUAGUCCGUUAUCAACUUGAAAAAGUGGCA |
| | | CCGAGUCGGUGCU(mU)*(mU)*U (SEQ ID: 67) |
| PSMD3 | PSMD3_g1 | (mC)*(mA)*CGAGCGCGAGAUAGCGUCGUUUUAGAGCUAGAAAUAG |
| | | CAAGUUAAAAUAAGGCUAGUCCGUUAUCAACUUGAAAAAGUGGCA |
| | | CCGAGUCGGUGCU(mU)*(mU)*U (SEQ ID: 68) |
| | PSMD3_g2 | (mA)*(mG)*CGUCGGGCCGCACGAUGAGUUUUAGAGCUAGAAAUAG |
| | | CAAGUUAAAAUAAGGCUAGUCCGUUAUCAACUUGAAAAAGUGGCA |
| | | CCGAGUCGGUGCU(mU)*(mU)*U (SEQ ID: 69) |
| | PSMD3_g3 | (mG)*(mC)*UCGUGUGCAGGCCCGGCUGUUUUAGAGCUAGAAAUAG |
| | | CAAGUUAAAAUAAGGCUAGUCCGUUAUCAACUUGAAAAAGUGGCA |
| | | CCGAGUCGGUGCU(mU)*(mU)*U (SEQ ID: 70) |
| PSMD8 | PSMD8_g1 | (mG)*(mG)*CGGCCGCGGCGGUGAACGGUUUUAGAGCUAGAAAUAG |
| | | CAAGUUAAAAUAAGGCUAGUCCGUUAUCAACUUGAAAAAGUGGCA |
| | | CCGAGUCGGUGCU(mU)*(mU)*U (SEQ ID: 71) |
| | PSMD8_g2 | (mU)*(mG)*CCGCAUCACGCAAGAUGGGUUUUAGAGCUAGAAAUAG |
| | | CAAGUUAAAAUAAGGCUAGUCCGUUAUCAACUUGAAAAAGUGGCA |
| | | CCGAGUCGGUGCU(mU)*(mU)*U (SEQ ID: 72) |
| | PSMD8_g3 | (mC)*(mG)*GCGCUGCCGUAAAUCAGGGUUUUAGAGCUAGAAAUAG |
| | | CAAGUUAAAAUAAGGCUAGUCCGUUAUCAACUUGAAAAAGUGGCA |
| | | CCGAGUCGGUGCU(mU)*(mU)*U (SEQ ID: 73) |
| PSMD11 | PSMD11_g1 | (mA)*(mC)*GGUGUGAGAGCGGUAAGAGUUUUAGAGCUAGAAAUAG |
| | | CAAGUUAAAAUAAGGCUAGUCCGUUAUCAACUUGAAAAAGUGGCA |
| | | CCGAGUCGGUGCU(mU)*(mU)*U (SEQ ID: 74) |
| | PSMD11_g2 | (mG)*(mG)*CCGGGGACGGUGUGAGAGGUUUUAGAGCUAGAAAUAG |
| | | CAAGUUAAAAUAAGGCUAGUCCGUUAUCAACUUGAAAAAGUGGCA |
| | | CCGAGUCGGUGCU(mU)*(mU)*U (SEQ ID: 75) |
| | PSMD1 | (mG)*(mU)*GUGAGAGCGGUAAGAUGGGUUUUAGAGCUAGAAAUAG |
| | | CAAGUUAAAAUAAGGCUAGUCCGUUAUCAACUUGAAAAAGUGGCA |
| | | CCGAGUCGGUGCU(mU)*(mU)*U (SEQ ID: 76) |
| VCP | VCP_g1 | (mG)*(mG)*CUCCGGAGUUUAUCCUCCGUUUUAGAGCUAGAAAUAG |
| | | CAAGUUAAAAUAAGGCUAGUCCGUUAUCAACUUGAAAAAGUGGCA |
| | | CCGAGUCGGUGCU(mU)*(mU)*U (SEQ ID: 77) |
| | VCP_g2 | (mG)*(mA)*GAAGGAGCAAGAAGUGUCGUUUUAGAGCUAGAAAUAG |
| | | CAAGUUAAAAUAAGGCUAGUCCGUUAUCAACUUGAAAAAGUGGCA |
| | | CCGAGUCGGUGCU(mU)*(mU)*U (SEQ ID: 78) |
| | VCP_g3 | (mC)*(mC)*GCGAGGUGGCAGUGGCAGGUUUUAGAGCUAGAAAUAG |
| | | CAAGUUAAAAUAAGGCUAGUCCGUUAUCAACUUGAAAAAGUGGCA |
| | | CCGAGUCGGUGCU(mU)*(mU)*U (SEQ ID: 79) |
| MRE11A | MRE11A_g1 | (mC)*(mU)*GAAUUCCGCGGGAGAGAAGUUUUAGAGCUAGAAAUAG |
| | | CAAGUUAAAAUAAGGCUAGUCCGUUAUCAACUUGAAAAAGUGGCA |
| | | CCGAGUCGGUGCU(mU)*(mU)*U (SEQ ID: 80) |
| | MRE11A_g2 | (mU)*(mC)*CGUGAAAAGAAAACAACAGUUUUAGAGCUAGAAAUAG |
| | | CAAGUUAAAAUAAGGCUAGUCCGUUAUCAACUUGAAAAAGUGGCA |
| | | CCGAGUCGGUGCU(mU)*(mU)*U (SEQ ID: 81) |
| | MRE11A_g3 | (mG)*(mG)*CCGUAAACCUGAAUUCCGGUUUUAGAGCUAGAAAUAG |
| | | CAAGUUAAAAUAAGGCUAGUCCGUUAUCAACUUGAAAAAGUGGCA |
| | | CCGAGUCGGUGCU(mU)*(mU)*U (SEQ ID: 82) |
| PSMA2 | PSMA2_g1 | (mG)*(mG)*GUAAAGAUGGCGGAGCGCGUUUUAGAGCUAGAAAUAG |
| | | CAAGUUAAAAUAAGGCUAGUCCGUUAUCAACUUGAAAAAGUGGCA |
| | | CCGAGUCGGUGCU(mU)*(mU)*U (SEQ ID: 83) |
| | PSMA2_g2 | (mG)*(mC)*UUUUCGCUGACUACAUUCGUUUUAGAGCUAGAAAUAG |
| | | CAAGUUAAAAUAAGGCUAGUCCGUUAUCAACUUGAAAAAGUGGCA |
| | | CCGAGUCGGUGCU(mU)*(mU)*U (SEQ ID: 84) |
| | PSMA2_g3 | (mG)*(mA)*CUACGCUGAAGACCUCGAGUUUUAGAGCUAGAAAUAG |
| | | CAAGUUAAAAUAAGGCUAGUCCGUUAUCAACUUGAAAAAGUGGCA |
| | | CCGAGUCGGUGCU(mU)*(mU)*U (SEQ ID: 85) |
| CD151 | CD151_g1 | (mC)*(mC)*CGGACUCGGACGCGUGGUGUUUUAGAGCUAGAAAUAG |
| | | CAAGUUAAAAUAAGGCUAGUCCGUUAUCAACUUGAAAAAGUGGCA |
| | | CCGAGUCGGUGCU(mU)*(mU)*U (SEQ ID: 86) |
| | CD151_g2 | (mG)*(mC)*GGCCCGGAGCCUACGAGGGUUUUAGAGCUAGAAAUAG |
| | | CAAGUUAAAAUAAGGCUAGUCCGUUAUCAACUUGAAAAAGUGGCA |
| | | CCGAGUCGGUGCU(mU)*(mU)*U (SEQ ID: 87) |
| | CD151_g3 | (mA)*(mG)*GGCCCGGACUCGGACGCGGUUUUAGAGCUAGAAAUAG |
| | | CAAGUUAAAAUAAGGCUAGUCCGUUAUCAACUUGAAAAAGUGGCA |
| | | CCGAGUCGGUGCU(mU)*(mU)*U (SEQ ID: 88) |
| SETD3 | SETD3_g1 | (mA)*(mA)*CCAACCCCCAGGCGGUGGGUUUUAGAGCUAGAAAUAG |
| | | CAAGUUAAAAUAAGGCUAGUCCGUUAUCAACUUGAAAAAGUGGCA |
| | | CCGAGUCGGUGCU(mU)*(mU)*U (SEQ ID: 89) |
| | SETD3_g2 | (mC)*(mU)*CAACCAACCCCCAGGCGGGUUUUAGAGCUAGAAAUAG |
| | | CAAGUUAAAAUAAGGCUAGUCCGUUAUCAACUUGAAAAAGUGGCA |
| | | CCGAGUCGGUGCU(mU)*(mU)*U (SEQ ID: 90) |
| | SETD3_g3 | (mC)*(mC)*UCGCAGAGCUCGGAGACGGUUUUAGAGCUAGAAAUAG |
| | | CAAGUUAAAAUAAGGCUAGUCCGUUAUCAACUUGAAAAAGUGGCA |
| | | CCGAGUCGGUGCU(mU)*(mU)*U (SEQ ID: 91) |
| TFRC | TFRC_g1 | (mG)*(mC)*AGCCAUAGGGAGCCGCACGUUUUAGAGCUAGAAAUAG |
| | | CAAGUUAAAAUAAGGCUAGUCCGUUAUCAACUUGAAAAAGUGGCA |
| | | CCGAGUCGGUGCU(mU)*(mU)*U (SEQ ID: 92) |
| | TFRC_g2 | (mG)*(mG)*AUGGCGGCCCCUAACCGGGUUUUAGAGCUAGAAAUAG |
| | | CAAGUUAAAAUAAGGCUAGUCCGUUAUCAACUUGAAAAAGUGGCA |
| | | CCGAGUCGGUGCU(mU)*(mU)*U (SEQ ID: 93) |
| | TFRC_g3 | (mC)*(mA)*GAGCGUCGGGAUAUCGGGGUUUUAGAGCUAGAAAUAG |
| | | CAAGUUAAAAUAAGGCUAGUCCGUUAUCAACUUGAAAAAGUGGCA |
| | | CCGAGUCGGUGCU(mU)*(mU)*U (SEQ ID: 94) |
| LBR | LBR_g1 | (mA)*(mU)*AGUCGCACAGCAACCCGGGUUUUAGAGCUAGAAAUAG |
| | | CAAGUUAAAAUAAGGCUAGUCCGUUAUCAACUUGAAAAAGUGGCA |
| | | CCGAGUCGGUGCU(mU)*(mU)*U (SEQ ID: 95) |
| | LBR_g2 | (mA)*(mG)*AAUAGUCGCACAGCAACCGUUUUAGAGCUAGAAAUAG |
| | | CAAGUUAAAAUAAGGCUAGUCCGUUAUCAACUUGAAAAAGUGGCA |
| | | CCGAGUCGGUGCU(mU)*(mU)*U (SEQ ID: 96) |
| | LBR_g3 | (mG)*(mG)*UUCCGGCGGUGACACGGAGUUUUAGAGCUAGAAAUAG |
| | | CAAGUUAAAAUAAGGCUAGUCCGUUAUCAACUUGAAAAAGUGGCA |
| | | CCGAGUCGGUGCU(mU)*(mU)*U (SEQ ID: 97) |
| XRCC4 | XRCC4_g1 | (mC)*(mC)*GGAAGUAGAGUCACGGAGGUUUUAGAGCUAGAAAUAG |
| | | CAAGUUAAAAUAAGGCUAGUCCGUUAUCAACUUGAAAAAGUGGCA |
| | | CCGAGUCGGUGCU(mU)*(mU)*U (SEQ ID: 98) |
| | XRCC4_g2 | (mA)*(mG)*AGGUAGGAUCCGGAAGUGGUUUUAGAGCUAGAAAUAG |
| | | CAAGUUAAAAUAAGGCUAGUCCGUUAUCAACUUGAAAAAGUGGCA |
| | | CCGAGUCGGUGCU(mU)*(mU)*U (SEQ ID: 99) |
| | XRCC4_g3 | (mA)*(mG)*AUACCGGAAGUAGAGUCAGUUUUAGAGCUAGAAAUAG |
| | | CAAGUUAAAAUAAGGCUAGUCCGUUAUCAACUUGAAAAAGUGGCA |
| | | CCGAGUCGGUGCU(mU)*(mU)*U (SEQ ID: 100) |
| CXCR3 | CXCR3_g1 | (mU)*(mU)*ACCUCAAGGACCAUGGCUGUUUUAGAGCUAGAAAUAG |
| | | CAAGUUAAAAUAAGGCUAGUCCGUUAUCAACUUGAAAAAGUGGCA |
| | | CCGAGUCGGUGCU(mU)*(mU)*U (SEQ ID: 101) |
| | CXCR3_g2 | (mG)*(mG)*GCAGCAGCACUUACCUCAGUUUUAGAGCUAGAAAUAG |
| | | CAAGUUAAAAUAAGGCUAGUCCGUUAUCAACUUGAAAAAGUGGCA |
| | | CCGAGUCGGUGCU(mU)*(mU)*U (SEQ ID: 102) |
| | CXCR3_g3 | (mC)*(mC)*ACAAGCACCAAAGCAGAGGUUUUAGAGCUAGAAAUAG |
| | | CAAGUUAAAAUAAGGCUAGUCCGUUAUCAACUUGAAAAAGUGGCA |
| | | CCGAGUCGGUGCU(mU)*(mU)*U (SEQ ID: 103) |
| LBR | 20 nt | (mG)*(mC)*CGAUGGUGAAGUGGUAAGGUUUUAGAGCUAGAAAUAG |
| | LBR_g2 | CAAGUUAAAAUAAGGCUAGUCCGUUAUCAACUUGAAAAAGUGGCA |
| | | CCGAGUCGGUGCU(mU)*(mU)*U (SEQ ID: 104) |
| PPIB | 20 nt | (mG)*(mU)*GUAUUUUGACCUACGAAUGUUUUAGAGCUAGAAAUAG |
| | PPIB_g2 | CAAGUUAAAAUAAGGCUAGUCCGUUAUCAACUUGAAAAAGUGGCA |
| | | CCGAGUCGGUGCU(mU)*(mU)*U (SEQ ID: 105) |

TABLE 3
5′ truncated 14 mer targeting region synthetic sgRNAs (SpCas9)
Target region is bolded, chemical modifications are italicized.

| Target | Guide Name | SEQUENCE |
|---|---|---|
| NTC | 14 nt | (mC)*(mG)*AACUACGCGGGUGUUUUAGAGCUAGAAAUAGCAAGUUAA |
| | NTC | AAUAAGGCUAGUCCGUUAUCAACUUGAAAAAGUGGCACCGAGUCGGUGC |
| | | U(mU)*(mU)*U (SEQ ID: 106) |
| LBR | 14 nt | (mG)*(mC)*ACAGCAACCCGGGUUUUAGAGCUAGAAAUAGCAAGUUAA |
| | LBR_g1 | AAUAAGGCUAGUCCGUUAUCAACUUGAAAAAGUGGCACCGAGUCGGUGC |
| | | U(mU)*(mU)*U (SEQ ID: 107) |
| | 14nt | (mG)*(mU)*CGCACAGCAACCGUUUUAGAGCUAGAAAUAGCAAGUUAA |
| | LBR_g2 | AAUAAGGCUAGUCCGUUAUCAACUUGAAAAAGUGGCACCGAGUCGGUGC |
| | | U(mU)*(mU)*U (SEQ ID: 108) |
| | 14nt | (mG)*(mG)*CGGUGACACGGAGUUUUAGAGCUAGAAAUAGCAAGUUAA |
| | LBR_g3 | AAUAAGGCUAGUCCGUUAUCAACUUGAAAAAGUGGCACCGAGUCGGUGC |
| | | U(mU)*(mU)*U (SEQ ID: 109) |
| MRE11A | 14 nt | (mU)*(mC)*CGCGGGAGAGAAGUUUUAGAGCUAGAAAUAGCAAGUUAA |
| | MRE11A_g1 | AAUAAGGCUAGUCCGUUAUCAACUUGAAAAAGUGGCACCGAGUCGGUGC |
| | | U(mU)*(mU)*U (SEQ ID: 110) |
| | 14 nt | (mA)*(mA)*AAGAAAACAACAGUUUUAGAGCUAGAAAUAGCAAGUUAA |
| | MRE11A_g2 | AAUAAGGCUAGUCCGUUAUCAACUUGAAAAAGUGGCACCGAGUCGGUGC |
| | | U(mU)*(mU)*U (SEQ ID: 111) |
| | 14 nt | (mA)*(mA)*ACCUGAAUUCCGGUUUUAGAGCUAGAAAUAGCAAGUUAA |
| | MRE11A_g3 | AAUAAGGCUAGUCCGUUAUCAACUUGAAAAAGUGGCACCGAGUCGGUGC |
| | | U(mU)*(mU)*U (SEQ ID: 112) |
| SEL1L | 14 nt | (mC)*(mG)*GAUACUGACCCGGUUUUAGAGCUAGAAAUAGCAAGUUAA |
| | SEL1L_g1 | AAUAAGGCUAGUCCGUUAUCAACUUGAAAAAGUGGCACCGAGUCGGUGC |
| | | U(mU)*(mU)*U (SEQ ID: 113) |
| | 14 nt | (mA)*(mC)*CCGAGGACGCCGGUUUUAGAGCUAGAAAUAGCAAGUUAA |
| | SEL1L_g2 | AAUAAGGCUAGUCCGUUAUCAACUUGAAAAAGUGGCACCGAGUCGGUGC |
| | | U(mU)*(mU)*U (SEQ ID: 114) |
| | 14 nt | (mG)*(mG)*CUGAGUCCGUGGGUUUUAGAGCUAGAAAUAGCAAGUUAA |
| | SEL1L_g3 | AAUAAGGCUAGUCCGUUAUCAACUUGAAAAAGUGGCACCGAGUCGGUGC |
| | | U(mU)*(mU)*U (SEQ ID: 115) |
| XRCC4 | 14 nt | (mG)*(mU)*AGAGUCACGGAGGUUUUAGAGCUAGAAAUAGCAAGUUAA |
| | XRCC4_g1 | AAUAAGGCUAGUCCGUUAUCAACUUGAAAAAGUGGCACCGAGUCGGUGC |
| | | U(mU)*(mU)*U (SEQ ID: 116) |
| | 14 nt | (mA)*(mG)*GAUCCGGAAGUGGUUUUAGAGCUAGAAAUAGCAAGUUAA |
| | XRCC4_g2 | AAUAAGGCUAGUCCGUUAUCAACUUGAAAAAGUGGCACCGAGUCGGUGC |
| | | U(mU)*(mU)*U (SEQ ID: 117) |
| | 14 nt | (mC)*(mG)*GAAGUAGAGUCAGUUUUAGAGCUAGAAAUAGCAAGUUAA |
| | XRCC4_g3 | AAUAAGGCUAGUCCGUUAUCAACUUGAAAAAGUGGCACCGAGUCGGUGC |
| | | U(mU)*(mU)*U (SEQ ID: 118) |
| | 14 nt | (mG)*(mG)*UGAAGUGGUAAGGUUUUAGAGCUAGAAAUAGCAAGUUAA |
| | LBR_g2 | AAUAAGGCUAGUCCGUUAUCAACUUGAAAAAGUGGCACCGAGUCGGUGC |
| | | U(mU)*(mU)*U (SEQ ID: 119) |
| | 14 nt | (mU)*(mU)*UGACCUACGAAUGUUUUAGAGCUAGAAAUAGCAAGUUAA |
| | PPIB_g2 | AAUAAGGCUAGUCCGUUAUCAACUUGAAAAAGUGGCACCGAGUCGGUGC |
| | | U(mU)*(mU)*U (SEQ ID: 120) |

TABLE 4
LNA modified synthetic sgRNAs (Sp Cas9)
Target region is bolded, chemical modifications are italicized.

| Target | Guide Name | SEQUENCE |
|---|---|---|
| PSMD7 | PSMD 7 | (G-LNA) (5mC- |
| | 2× 5′ | LNA)GCCGCCGGCCCAGCUAUAGUUUUAGAGCUAGAAAUAGCAAGUUAA |
| | LNA, | AAUAAGGCUAGUCCGUUAUCAACUUGAAAAAGUGGCACCGAGUCGGUGC |
| | 2x | U(mU)*(mU)*U (SEQ ID: 121) |
| | O′me | |
| | 3′ | |
| | sgRNA | |
| | PSMD 7 | (mG)*(mC)*GCCGCCGGCCCAGCUAUAGUUUUAGAGCUAGAAAUAGCA |
| | 2x 5′ | AGUUAAAAUAAGGCUAGUCCGUUAUCAACUUGAAAAAGUGGCACCGAGU |
| | O′me, | CGGUGCU(T-LNA)(T-LNA)U (SEQ ID: 122) |
| | 2x 3′ | |
| | LNA | |
| | sgRNA | |
| | PSMD7 | (G-LNA)(5mC- |
| | 2x | LNA)GCCGCCGGCCCAGCUAUAGUUUUAGAGCUAGAAAUAGCAAGUUAA |
| | LNA | AAUAAGGCUAGUCCGUUAUCAACUUGAAAAAGUGGCACCGAGUCGGUGC |
| | 5′ | U(T-LNA)(T-LNA)U (SEQ ID: 123) |
| | and | |
| | 3′ | |
| | sgRNA | |
| PSMD11 | PSMD1 | (G-LNA)(T- |
| | 1 2x | LNA)GUGAGAGCGGUAAGAUGGGUUUUAGAGCUAGAAAUAGCAAGUUAA |
| | 5′ | AAUAAGGCUAGUCCGUUAUCAACUUGAAAAAGUGGCACCGAGUCGGUGC |
| | LNA, | U(mU*)(mU*)U (SEQ ID: 124) |
| | 2x | |
| | O′me | |
| | 3′ | |
| | sgRNA | |
| | PSMD1 | (mG)*(mU)*GUGAGAGCGGUAAGAUGGGUUUUAGAGCUAGAAAUAGCA |
| | 1 2x | AGUUAAAAUAAGGCUAGUCCGUUAUCAACUUGAAAAAGUGGCACCGAGU |
| | 5′ | CGGUGCU(T-LNA)(T-LNA)U (SEQ ID: 125) |
| | O′me, | |
| | 2x 3′ | |
| | LNA | |
| | sgRNA | |
| | PSMD1 | (G-LNA)(T- |
| | 1 2x | LNA)GUGAGAGCGGUAAGAUGGGUUUUAGAGCUAGAAAUAGCAAGUUAA |
| | LNA | AAUAAGGCUAGUCCGUUAUCAACUUGAAAAAGUGGCACCGAGUCGGUGC |
| | 5′ | U(T-LNA)(T-LNA)U (SEQ ID: 126) |
| | and | |
| | 3′ | |
| | sgRNA | |
| PSMD7 | PSMD7 | (mG)*(mC)*GCC(G- |
| | LNA | LNA)CCGGCCCAGCUAUAGUUUUAGAGCUAGAAAUAGCAAGUUAAAAUA |
| | position 6 | AGGCUAGUCCGUUAUCAACUUGAAAAAGUGGCACCGAGUCGGUGCU(mU)* |
| | | (mU)*U (SEQ ID: 127) |
| | PSMD 7 | (mG)*(mC)*GCCGCCG(G- |
| | LNA | LNA)CCCAGCUAUAGUUUUAGAGCUAGAAAUAGCAAGUUAAAAUAAGGC |
| | position | UAGUCCGUUAUCAACUUGAAAAAGUGGCACCGAGUCGGUGCU(mU)* |
| | 10 | (mU)*U (SEQ ID: 128) |
| | PSMD 7 | (mG)*(mC)*GCCGCCGGC(5mC- |
| | LNA | LNA)CAGCUAUAGUUUUAGAGCUAGAAAUAGCAAGUUAAAAUAAGGCUA |
| | position | GUCCGUUAUCAACUUGAAAAAGUGGCACCGAGUCGGUGCU(mU)*(mU) |
| | 12 | *U (SEQ ID: 129) |
| PSMD11 | PSMD1 | (mG)*(mU)*GUG(A- |
| | 1 LNA | LNA)GAGCGGUAAGAUGGGUUUUAGAGCUAGAAAUAGCAAGUUAAAAUA |
| | position 6 | AGGCUAGUCCGUUAUCAACUUGAAAAAGUGGCACCGAGUCGGUGCU(mU*) |
| | | (mU*)U (SEQ ID: 130) |
| | PSMD1 | (mG)*(mU)*GUGAGAG(5mC- |
| | 1 LNA | LNA)GGUAAGAUGGGUUUUAGAGCUAGAAAUAGCAAGUUAAAAUAAGGC |
| | position | UAGUCCGUUAUCAACUUGAAAAAGUGGCACCGAGUCGGUGCU(mU*)(mU*)U |
| | 10 | (SEQ ID: 131) |
| | PSMD1 | (mG)*(mU)*GUGAGAGCG(G- |
| | 1 LNA | LNA)UAAGAUGGGUUUUAGAGCUAGAAAUAGCAAGUUAAAAUAAGGCUA |
| | position | GUCCGUUAUCAACUUGAAAAAGUGGCACCGAGUCGGUGCU(mU*)(mU*)U |
| | 12 | (SEQ ID: 132) |

TABLE 5
synthetic crRNAs (Sp Cas9)
Target region is bolded, chemical modifications are italicized.

| Target | Guide Name | SEQUENCE |
|---|---|---|
| NTC | NTC | (mG)*(mU)*AACGCGAACUACGCGGGUGUUUUAGAGCUAUGCUGUU |
| | (crRNA) | UUG (SEQ ID: 133) |
| CD151 | CD151_cr1 | (mC)*(mC)*CGGACUCGGACGCGUGGUGUUUUAGAGCUAUGCUGUU |
| | | UUG (SEQ ID: 134) |
| | CD151_cr2 | (mG)*(mC)*GGCCCGGAGCCUACGAGGGUUUUAGAGCUAUGCUGUU |
| | | UUG (SEQ ID: 135) |
| | CD151_cr3 | (mA)*(mG)*GGCCCGGACUCGGACGCGGUUUUAGAGCUAUGCUGUU |
| | | UUG (SEQ ID: 136) |
| SEL1L | SEL1L_cr1 | (mA)*(mG)*GGGGCGGAUACUGACCCGGUUUUAGAGCUAUGCUGUU |
| | | UUG (SEQ ID: 137) |
| | SEL1L_cr2 | (mA)*(mU)*ACUGACCCGAGGACGCCGGUUUUAGAGCUAUGCUGUU |
| | | UUG (SEQ ID: 138) |
| | SEL1L_cr3 | (mG)*(mG)*UGGUGGCUGAGUCCGUGGGUUUUAGAGCUAUGCUGUU |
| | | UUG (SEQ ID: 139) |
| SETD3 | SETD3_cr1 | (mA)*(mA)*CCAACCCCCAGGCGGUGGGUUUUAGAGCUAUGCUGUU |
| | | UUG (SEQ ID: 140) |
| | SETD3_cr2 | (mC)*(mU)*CAACCAACCCCCAGGCGGGUUUUAGAGCUAUGCUGUU |
| | | UUG (SEQ ID: 141) |
| | SETD3_cr3 | (mC)*(mC)*UCGCAGAGCUCGGAGACGGUUUUAGAGCUAUGCUGUU |
| | | UUG (SEQ ID: 142) |
| TFRC | TFRC_cr1 | (mG)*(mC)*AGCCAUAGGGAGCCGCACGUUUUAGAGCUAUGCUGUU |
| | | UUG (SEQ ID: 143) |
| | TFRC_cr2 | (mG)*(mG)*AUGGCGGCCCCUAACCGGGUUUUAGAGCUAUGCUGUU |
| | | UUG (SEQ ID: 144) |
| | TFRC_cr3 | (mC)*(mA)*GAGCGUCGGGAUAUCGGGGUUUUAGAGCUAUGCUGUU |
| | | UUG (SEQ ID: 145) |

TABLE 6
synthetic tracrRNAs (Sp Cas9)
MS2 aptamer region is bolded, chemical modifications are italicized.

| Traer Name | SEQUENCE |
|---|---|
| tracrRNA w/out | AACAGCAUAGCAAGUUAAAAUAAGGCUAGUCCGUUAUCAACUUGA |
| MS2 | AAAAGUGGCACCGAGUCGGUGCUUUU(mU)*(mU)*U (SEQ ID: 146) |
| stem loop 2 C-5 | AACAGCAUAGCAAGUUAAAAUAAGGCUAGUCCGUUAUCAACUUGG |
| MS2 tracrRNA | CCAACAUGAGGAUCACCCAUGUCUGCAGGGCCAAGUGGCACCGA |
| | GUCGGUGCUUUU(mU)*(mU)*U (SEQ ID: 147) |
| 3′ C-5 MS2 | AACAGCAUAGCAAGUUAAAAUAAGGCUAGUCCGUUAUCAACUUGA |
| tracrRNA | AAAAGUGGCACCGAGUCGGUGCGCGCACAUGAGGAUCACCCAUGU |
| | GCUUUU(mU)*(mU)*U (SEQ ID: 148) |
| 3′ chemically | AACAGCAUAGCAAGUUAAAAUAAGGCUAGUCCGUUAUCAACUUGA |
| enhanced 3′ F-5 | AAAAGUGGCACCGAGUCGGUGCGCGGCCCGG-(2AdP)- |
| MS2 tracrRNA | GGAUCACCACGGGCCUUUU(mU)*(mU)*U (SEQ ID: 149) |

TABLE 7
synthetic Class V Cas crRNAs
Target region is bolded, chemical modifications are italicized.

| Target | Guide Name | SEQUENCE |
|---|---|---|
| NTC | NTC | UUAAUUUCUACUCUUGUAGAUAGAGUGCCUAGAAAGAUGACA |
| | (dMAD7) | (SEQ ID: 150) |
| BRCA1 | dMAD7 | UUAAUUUCUACUCUUGUAGAUCUGGACGGGGGACAGGCUGUG |
| | BRCA1_g2 | (SEQ ID: 151) |
| | dMAD7 | UUAAUUUCUACUCUUGUAGAUUCAGAUAACUGGGCCCCUGCG |
| | BRCA1_g4 | (SEQ ID: 152) |
| PPIB | dMAD7 | UUAAUUUCUACUCUUGUAGAUCCCCCUCCGGCUCGGCGCCGG |
| | PPIB_g2 | (SEQ ID: 153) |
| | dMAD7 | UUAAUUUCUACUCUUGUAGAUGCCUCCGCCUGUGGAUGCUGC |
| | PPIB_g3 | (SEQ ID: 154) |
| NTC | NTC | (mC)*(mU)*UUCAAGACUAAUAGAUUGCUCCUUACGAGGAGACAGAG |
| | (dCas | UGCCUAGAAAG(mA)*(mU)*G (SEQ ID: 155) |
| | Phi8) | |
| BRCA1 | pre- | (mC)*(mU)*UUCAAGACUAAUAGAUUGCUCCUUACGAGGAGACCUGG |
| | crRNA | ACGGGGGACAG(mG)*(mC)*U (SEQ ID: 156) |
| | w/18 nt | |
| | spacer | |
| | crRNA | (mA)*(mA)*UAGAUUGCUCCUUACGAGGAGACCUGGACGGGGGACAG |
| | only | (mG)*(mC)*U (SEQ ID: 157) |
| | w/18 nt | |
| | spacer | |
| | crRNA | (mA)*(mA)*UAGAUUGCUCCUUACGAGGAGACCUGGACGGGGG(mA)* |
| | only w/ | (mC)*A (SEQ ID: 158) |
| | 14 nt | |
| | spacer | |

TABLE 8
Lentiviral guide RNAs (Sp Cas9) delivered via particles or as plasmids
Target region is bolded

| Target | Guide Name | SEQUENCE |
|---|---|---|
| NTC | LV | GTAACGCGAACTACGCGGGTGTTTAAGAGCTATGCTGGAAACAGCA |
| | NTC | TAGCAAGTTTAAATAAGGCTAGTCCGTTATCAACTTGAAAAAGTGGC |
| | | ACCGAGTCGGTGCTTTTTTT (SEQ ID: 159) |
| CD46 | LV | GGATTGTTGCGTCCCATATCGTTTAAGAGCTATGCTGGAAACAGCA |
| | CD46 | TAGCAAGTTTAAATAAGGCTAGTCCGTTATCAACTTGAAAAAGTGGC |
| | g2 | ACCGAGTCGGTGCTTTTTTT (SEQ ID: 160) |
| | LV | GCTCAGTCGGGCAAGAGTCGGTTTAAGAGCTATGCTGGAAACAGCA |
| | CD46 | TAGCAAGTTTAAATAAGGCTAGTCCGTTATCAACTTGAAAAAGTGGC |
| | g4 | ACCGAGTCGGTGCTTTTTTT (SEQ ID: 161) |
| PSMD7 | LV | GACTGGGCCTGAAAGGGTACGTTTAAGAGCTATGCTGGAAACAGCA |
| | PSMD7 | TAGCAAGTTTAAATAAGGCTAGTCCGTTATCAACTTGAAAAAGTGGC |
| | g1 | ACCGAGTCGGTGCTTTTTTT (SEQ ID: 162) |
| | LV | GCGCCGCCGGCCCAGCTATAGTTTAAGAGCTATGCTGGAAACAGCA |
| | PSMD7 | TAGCAAGTTTAAATAAGGCTAGTCCGTTATCAACTTGAAAAAGTGGC |
| | g2 | ACCGAGTCGGTGCTTTTTTT (SEQ ID: 163) |
| | LV | GCGGCAGCAGTAGCGGTCACGTTTAAGAGCTATGCTGGAAACAGCA |
| | PSMD7 | TAGCAAGTTTAAATAAGGCTAGTCCGTTATCAACTTGAAAAAGTGGC |
| | g4 | ACCGAGTCGGTGCTTTTTTT (SEQ ID: 164) |
| SEL1L | LV | GGGGGGCGGATACTGACCCGGTTTAAGAGCTATGCTGGAAACAGC |
| | SEL1L | ATAGCAAGTTTAAATAAGGCTAGTCCGTTATCAACTTGAAAAAGTGG |
| | g1 | CACCGAGTCGGTGCTTTTTTT (SEQ ID: 165) |
| | LV | GGTGGTGGCTGAGTCCGTGGGTTTAAGAGCTATGCTGGAAACAGCA |
| | SEL1L | TAGCAAGTTTAAATAAGGCTAGTCCGTTATCAACTTGAAAAAGTGGC |
| | g3 | ACCGAGTCGGTGCTTTTTTT (SEQ ID: 166) |
| PPIB | LV | GGGAGAGGCGCAGCATCCACGTTTAAGAGCTATGCTGGAAACAGC |
| | PPIB g1 | ATAGCAAGTTTAAATAAGGCTAGTCCGTTATCAACTTGAAAAAGTGG |
| | | CACCGAGTCGGTGCTTTTTTT (SEQ ID: 167) |
| BRCA1 | LV | GCACAGCCTGTCCCCCGTCCGTTTAAGAGCTATGCTGGAAACAGCA |
| | BRCA1 | TAGCAAGTTTAAATAAGGCTAGTCCGTTATCAACTTGAAAAAGTGGC |
| | g3 | ACCGAGTCGGTGCTTTTTTT (SEQ ID: 168) |
| ST3G | LV | GATCCGCTAGGCGCACCGACGTTTAAGAGCTATGCTGGAAACAGCA |
| AL4 | ST3GA | TAGCAAGTTTAAATAAGGCTAGTCCGTTATCAACTTGAAAAAGTGGC |
| | LA g2 | ACCGAGTCGGTGCTTTTTTT (SEQ ID: 169) |

1. A Cas fusion protein comprising a Cas protein and one or both of a SALL1 repressor domain and a SUDS3 repressor domain.

2. (canceled)

3. (canceled)

4. The Cas fusion protein of claim 1, wherein the Cas fusion protein comprises both the SALL1 repressor domain and the SUDS3 repressor domain.

5. The Cas fusion protein of claim 1, further comprising an additional repressor domain, wherein the additional repressor domain is a repressor domain other than the SALL1 repressor domain or the SUDS3 repressor domain.

6. The Cas fusion protein of claim 5, wherein the additional repressor domain is a NIPP1 repressor domain.

7. The Cas fusion protein of claim 1, wherein the Cas protein is catalytically inactive.

8. The Cas fusion protein of claim 1, wherein the Cas protein is a nickase.

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. The Cas fusion protein of claim 4, wherein the SALL1 repressor domain comprises a sequence that is at least 80% similar to SEQ ID NO: 1 and the SUDS3 repressor domain comprises a sequence that is at least 80% similar to SEQ ID NO: 2.

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. A nucleic acid encoding the Cas fusion protein of claim 1.

45. (canceled)

46. (canceled)

47. (canceled)

48. A method of modulating expression of a target nucleic acid in a eukaryotic cell comprising providing to the cell a gRNA and a Cas fusion protein of claim 1.

49. (canceled)

50. The method of claim 48, wherein the eukaryotic cell is a yeast cell, a plant cell or a mammalian cell.

51. (canceled)

52. (canceled)

53. (canceled)

54. (canceled)

55. A method of modulating expression of a target nucleic acid in a eukaryotic cell, said method comprising providing to the cell a Cas fusion protein of claim 1 and an RNA-repressor domain complex, wherein the RNA-repressor domain complex comprises:
(a) a gRNA molecule, wherein the gRNA molecule contains 30 to 180 nucleotides;
(b) a ligand binding moiety, wherein the ligand binding moiety is either (i) directly bound to the gRNA molecule, or (ii) bound through a ligand binding moiety linker to the gRNA molecule;
(c) a ligand, wherein the ligand is capable of reversibly associating with the ligand binding moiety; and
(d) a repressor domain, wherein the repressor domain is either (i) directly bound to the ligand, or (ii) bound through a ligand linker to the ligand.

56. (canceled)

57. A kit comprising a Cas fusion protein of claim 1 and an RNA-repressor domain complex, wherein the RNA-repressor domain complex comprises:
(a) a gRNA molecule, wherein the gRNA molecule contains 30 to 180 nucleotides;
(b) a ligand binding moiety, wherein the ligand binding moiety is either (i) directly bound to the gRNA molecule, or (ii) bound through a ligand binding moiety linker to the gRNA molecule;
(c) a ligand, wherein the ligand is capable of reversibly associating with the ligand binding moiety; and
(d) a repressor domain and wherein the repressor domain is either (i) directly bound to the ligand, or (ii) bound through a ligand linker to the ligand.

58. A RNA-repressor domain complex, wherein the RNA-repressor domain complex comprises:
(a) a gRNA molecule, wherein the gRNA molecule contains 30 to 180 nucleotides;
(b) a ligand binding moiety, wherein the ligand binding moiety is either (i) directly bound to the gRNA molecule, or (ii) bound through a ligand binding moiety linker to the gRNA molecule;
(c) a ligand, wherein the ligand is capable of reversibly associating with the ligand binding moiety; and
(d) a fusion protein, wherein the fusion protein comprises a SALL1 repressor domain and a SUDS3 repressor domain, and wherein the fusion protein is either (i) directly bound to the ligand, or (ii) bound through a ligand linker to the ligand.

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. The RNA-repressor domain complex of claim 58, wherein the ligand is selected from the group consisting of: MS2, Ku, PP7, SfMu, Sm7, Tat, Glutathione S-transferase (GST), CSY4, Qbeta, COM, pumilio, lambda N22, and PDGF beta-chain.

84. (canceled)

85. (canceled)

86. (canceled)

87. A method for transcriptional repression comprising exposing the RNA-repressor domain complex of claim 58 to double-stranded DNA.

88. (canceled)

89. (canceled)

90. (canceled)

91. A kit comprising the RNA-repressor domain complex of claim 83.

92. A method of treating a subject, said method comprising administering a Cas fusion protein of claim 1 to the subject.

93. (canceled)

94. (canceled)

95. A method of treating a subject, said method comprising administering a repressor domain complex of claim 58 to the subject.

96. (canceled)

97. (canceled)

98. (canceled)

99. (canceled)

100. A method of modulating expression of a target nucleic acid in a cell comprising providing to the cell a sgRNA and a Cas fusion protein of claim 1.

101. (canceled)

102. (canceled)

103. A method of modulating expression of a target nucleic acid in a cell comprising providing to the cell a crRNA molecule, a tracrRNA molecule and a Cas fusion protein of claim 1.

104. (canceled)

105. (canceled)


SEQUENCE LISTING (184 sequences)

SEQ ID NO: 1 - protein, 183 aa, Homo sapiens
MSRRKQAKPQHFQSDPEVASLPRRDGDTEKGQPSRPTKSKDAHVCGRCCAEFFELSDLLLHKKNCTKNQLVLIVNENPASPPETFSPSPPPDNPDEQMNDTVNKTDQVDCSDLSEHNGLDREESMEVEAPVANKSGSGTSSGSHSSTAPSSSSSSSSSSGGGGSSSTGTSAITTSLPQLGDLT

SEQ ID NO: 2 - protein, 328 aa, Homo sapiens
MSAAGLLAPAPAQAGAPPAPEYYPEEDEELESAEDDERSCRGRESDEDTEDASETDLAKHDEEDYVEMKEQMYQDKLASLKRQLQQLQEGTLQEYQKRMKKLDQQYKERIRNAELFLQLETEQVERNYIKEKKAAVKEFEDKKVELKENLIAELEEKKKMIENEKLTMELTGDSMEVKPIMTRKLRRRPNDPVPIPDKRRKPAPAQLNYLLTDEQIMEDLRTLNKLKSPKRPASPSSPEHLPATPAESPAQRFEARIEDGKLYYDKRWYHKSQAIYLESKDNQKLSCVISSVGANEIWVRKTSDSTKMRIYLGQLQRGLFVIRRRSAA

SEQ ID NO: 3 - protein, 127 aa, Homo sapiens
MVQTAVVPVKKKRVEGPGSLGLEESGSRRMQNFAFSGGLYGGLPPTHSEAGSQPHGIHGTALIGGLPMPYPNLAPDVDLTPVVPSAVNMNPAPNPAVYNPEAVNEPKKKKYAKEAWPGKKPTPSLLI

SEQ ID NO: 4 - DNA, 1566 bp, Artificial Sequence
  SALL1 represso domain nucleotide sequence
atgagtaggagaaaacaagcaaaaccacagcactttcaaagtgatcctgaggtagcaagccttccacggcgggacggtgacacggagaagggtcaaccaagtcgacccacgaaaagcaaagatgctcatgtatgtggacgctgttgcgcagaattttttgaattgtccgatcttcttcttcacaaaaagaactgcacgaagaatcagttggttttgatagtaaacgaaaatccagcttcacccccagaaactttttccccgtcacctcctccagataatcctgatgaacaaatgaatgacaccgtaaataaaaccgaccaagtagactgttctgatttgagcgaacacaacggtttggatcgagaagagtcaatggaagtagaggccccagttgccaataagtcaggcagcggtacttcttccggctcccacagttcaacagctccatcctcaagtagttcaagctcttctagttcaggaggcggggggagtagctctaccggcacttctgccatcacaacctcacttcctcagcttggagacttgacaggatccggtgggggatctgggggatctggctcgatgtctgcagctggccttttggctcctgcccccgcacaagcgggagctcctcccgcaccggagtactatccagaagaggatgaggaactggaatctgccgaagacgacgagcgcagttgccgggggagggaatctgacgaggatactgaggatgcttctgagaccgacctcgcgaaacatgatgaggaagactacgttgaaatgaaagagcagatgtaccaagacaaacttgctagcctcaagagacagttgcagcaactgcaagaaggcacgctccaggagtaccagaagagaatgaaaaaactcgaccagcagtacaaggaacgaattagaaacgcagagctctttcttcagctggagactgaacaggttgagcgcaattatattaaggaaaaaaaagccgctgtgaaggagttcgaagacaagaaagtggaacttaaagaaaacctcatcgccgaactggaggagaagaagaagatgatagagaacgaaaaactcacaatggaactgacgggtgattccatggaggtaaaaccgattatgacccgaaagctccgccgacgcccaaacgatccggtaccgatccctgataagcggcgcaagcccgcaccggctcagctcaattacctgctgaccgacgaacaaataatggaggacctgcggactcttaataagctgaagagtcctaaacggccagcttcccccagttcccccgaacacctgcccgctactcccgcggagagccctgctcagcgctttgaggcccgaatcgaggacggaaaattgtactatgacaaacgctggtatcataagagccaggctatatacctggagtcaaaagataaccaaaagttgtcatgtgtaatctcctcagtcggggctaacgaaatatgggtgcggaagacctctgatagtacgaagatgcgcatatatctgggacaattgcaaagaggactttttgttataagacggagaagcgctgct

SEQ ID NO: 5 - DNA, 984 bp, Artificial Sequence
  SUDS3 repressor domain nucleotide sequence
atgtctgcagctggccttttggctcctgcccccgcacaagcgggagctcctcccgcaccggagtactatccagaagaggatgaggaactggaatctgccgaagacgacgagcgcagttgccgggggagggaatctgacgaggatactgaggatgcttctgagaccgacctcgcgaaacatgatgaggaagactacgttgaaatgaaagagcagatgtaccaagacaaacttgctagcctcaagagacagttgcagcaactgcaagaaggcacgctccaggagtaccagaagagaatgaaaaaactcgaccagcagtacaaggaacgaattagaaacgcagagctctttcttcagctggagactgaacaggttgagcgcaattatattaaggaaaaaaaagccgctgtgaaggagttcgaagacaagaaagtggaacttaaagaaaacctcatcgccgaactggaggagaagaagaagatgatagagaacgaaaaactcacaatggaactgacgggtgattccatggaggtaaaaccgattatgacccgaaagctccgccgacgcccaaacgatccggtaccgatccctgataagcggcgcaagcccgcaccggctcagctcaattacctgctgaccgacgaacaaataatggaggacctgcggactcttaataagctgaagagtcctaaacggccagcttcccccagttcccccgaacacctgcccgctactcccgcggagagccctgctcagcgctttgaggcccgaatcgaggacggaaaattgtactatgacaaacgctggtatcataagagccaggctatatacctggagtcaaaagataaccaaaagttgtcatgtgtaatctcctcagtcggggctaacgaaatatgggtgcggaagacctctgatagtacgaagatgcgcatatatctgggacaattgcaaagaggactttttgttataagacggagaagcgctgct

SEQ ID NO: 6 - DNA, 381 bp, Artificial Sequence
  NIPP1 repressor domain nucleotide sequence
atggtgcaaactgcagtggtcccagtcaagaagaagcgtgtggagggccctggctccctgggcctggaggaatcagggagcaggcgcatgcagaactttgccttcagcggaggactctacgggggcctgccccccacacacagtgaagcaggctcccagccacatggcatccatgggacagcactcatcggtggcttgcccatgccatacccaaaccttgcccctgatgtggacttgactcctgttgtgccgtcagcagtgaacatgaaccctgcaccaaaccctgcagtctataaccctgaagctgtaaatgaacccaagaagaagaaatatgcaaaagaggcttggccaggcaagaagcccacaccttccttgctgatt

SEQ ID NO: 7 - protein, 11 aa, Artificial Sequence
  Synthetic
GSGGGSGGSGS

SEQ ID NO: 8 - DNA, 33 bp, Artificial Sequence
  Synthetic
ggatccggtgggggatctgggggatctggctcg

SEQ ID NO: 9 - DNA, 1599 bp, Artificial Sequence
  Synthetic
ggatccggtgggggatctgggggatctggctcgatgagtaggagaaaacaagcaaaaccacagcactttcaaagtgatcctgaggtagcaagccttccacggcgggacggtgacacggagaagggtcaaccaagtcgacccacgaaaagcaaagatgctcatgtatgtggacgctgttgcgcagaattttttgaattgtccgatcttcttcttcacaaaaagaactgcacgaagaatcagttggttttgatagtaaacgaaaatccagcttcacccccagaaactttttccccgtcacctcctccagataatcctgatgaacaaatgaatgacaccgtaaataaaaccgaccaagtagactgttctgatttgagcgaacacaacggtttggatcgagaagagtcaatggaagtagaggccccagttgccaataagtcaggcagcggtacttcttccggctcccacagttcaacagctccatcctcaagtagttcaagctcttctagttcaggaggcggggggagtagctctaccggcacttctgccatcacaacctcacttcctcagcttggagacttgacaggatccggtgggggatctgggggatctggctcgatgtctgcagctggccttttggctcctgcccccgcacaagcgggagctcctcccgcaccggagtactatccagaagaggatgaggaactggaatctgccgaagacgacgagcgcagttgccgggggagggaatctgacgaggatactgaggatgcttctgagaccgacctcgcgaaacatgatgaggaagactacgttgaaatgaaagagcagatgtaccaagacaaacttgctagcctcaagagacagttgcagcaactgcaagaaggcacgctccaggagtaccagaagagaatgaaaaaactcgaccagcagtacaaggaacgaattagaaacgcagagctctttcttcagctggagactgaacaggttgagcgcaattatattaaggaaaaaaaagccgctgtgaaggagttcgaagacaagaaagtggaacttaaagaaaacctcatcgccgaactggaggagaagaagaagatgatagagaacgaaaaactcacaatggaactgacgggtgattccatggaggtaaaaccgattatgacccgaaagctccgccgacgcccaaacgatccggtaccgatccctgataagcggcgcaagcccgcaccggctcagctcaattacctgctgaccgacgaacaaataatggaggacctgcggactcttaataagctgaagagtcctaaacggccagcttcccccagttcccccgaacacctgcccgctactcccgcggagagccctgctcagcgctttgaggcccgaatcgaggacggaaaattgtactatgacaaacgctggtatcataagagccaggctatatacctggagtcaaaagataaccaaaagttgtcatgtgtaatctcctcagtcggggctaacgaaatatgggtgcggaagacctctgatagtacgaagatgcgcatatatctgggacaattgcaaagaggactttttgttataagacggagaagcgctgct

SEQ ID NO: 10 - protein, 533 aa, Artificial Sequence
  Synthetic
GSGGGSGGSGSMSRRKQAKPQHFQSDPEVASLPRRDGDTEKGQPSRPTKSKDAHVCGRCCAEFFELSDLLLHKKNCTKNQLVLIVNENPASPPETFSPSPPPDNPDEQMNDTVNKTDQVDCSDLSEHNGLDREESMEVEAPVANKSGSGTSSGSHSSTAPSSSSSSSSSSGGGGSSSTGTSAITTSLPQLGDLTGSGGGSGGSGSMSAAGLLAPAPAQAGAPPAPEYYPEEDEELESAEDDERSCRGRESDEDTEDASETDLAKHDEEDYVEMKEQMYQDKLASLKRQLQQLQEGTLQEYQKRMKKLDQQYKERIRNAELFLQLETEQVERNYIKEKKAAVKEFEDKKVELKENLIAELEEKKKMIENEKLTMELTGDSMEVKPIMTRKLRRRPNDPVPIPDKRRKPAPAQLNYLLTDEQIMEDLRTLNKLKSPKRPASPSSPEHLPATPAESPAQRFEARIEDGKLYYDKRWYHKSQAIYLESKDNQKLSCVISSVGANEIWVRKTSDSTKMRIYLGQLQRGLFVIRRRSAA

SEQ ID NO: 11 - RNA, 100 nt, Artificial Sequence
  Synthetic
  misc_feature (1)..(2): Ribose contains 2′ O methyl
  misc_feature (1)..(3): linkage between nucleotides is phosphorothioate
  N_region (1)..(20): N is A, C, G, or U
  misc_feature (4)..(20): n is a, c, g, or u
  misc_feature (98)..(99): Ribose contains 2′ O methyl
  misc_feature (98)..(100): linkage between nucleotides is phosphorothioate
nnnnnnnnnnnnnnnnnnnnguuuuagagcuagaaauagcaaguuaaaauaaggcuaguccguuaucaacuugaaaaaguggcaccgagucggugcuuuu

SEQ ID NO: 12 - RNA, 66 nt, Artificial Sequence
  Ku binding hairpin
uucuugucguacuuauagaucgcuacguuauuucaauuuugaaaaucugaguccugggagugcgga

SEQ ID NO: 13 - protein, 609 aa, Artificial Sequence
  Ku heterodimer
MSGWESYYKTEGDEEAEEEQEENLEASGDYKYSGRDSLIFLVDASKAMFESQSEDELTPFDMSIQCIQSVYISKIISSDRDLLAVVFYGTEKDKNSVNFKNIYVLQELDNPGAKRILELDQFKGQQGQKRFQDMMGHGSDYSLSEVLWVCANLFSDVQFKMSHKRIMLFTNEDNPHGNDSAKASRARTKAGDLRDTGIFLDLMHLKKPGGFDISLFYRDIISIAEDEDLRVHFEESSKLEDLLRKVRAKETRKRALSRLKLKLNKDIVISVGIYNLVQKALKPPPIKLYRETNEPVKTKTRTFNTSTGGLLLPSDTKRSQIYGSRQIILEKEETEELKRFDDPGLMLMGFKPLVLLKKHHYLRPSLFVYPEESLVIGSSTLFSALLIKCLEKEVAALCRYTPRRNIPPYFVALVPQEEELDDQKIQVTPPGFQLVFLPFADDKRKMPFTEKIMATPEQVGKMKAIVEKLRFTYRSDSFENPVLQQHFRNLEALALDLMEPEQAVDLTLPKVEAMNKRLGSLVDEFKELVYPPDYNPEGKVTKRKHDNEGSGSKRPKVEYSEEELKTHISKGTLGKFTVPMLKEACRAYGLKSGLKKQELLEALTKHFQD

SEQ ID NO: 14 - protein, 485 aa, Artificial Sequence
  Ku heterodimer
MVRSGNKAAVVLCMDVGFTMSNSIPGIESPFEQAKKVITMFVQRQVFAENKDEIALVLFGTDGTDNPLSGGDQYQNITVHRHLMLPDFDLLEDIESKIQPGSQQADFLDALIVSMDVIQHETIGKKFEKRHIEIFTDLSSRFSKSQLDIIIHSLKKCDISERHSIHWPCRLTIGSNLSIRIAAYKSILQERVKKTWTVVDAKTLKKEDIQKETVYCLNDDDETEVLKEDIIQGFRYGSDIVPFSKVDEEQMKYKSEGKCFSVLGFCKSSQVQRRFFMGNQVLKVFAARDDEAAAVALSSLIHALDDLDMVAIVRYAYDKRANPQVGVAFPHIKHNYECLVYVQLPFMEDLRQYMFSSLKNSKKYAPTEAQLNAVDALIDSMSLAKKDEKTDTLEDLFPTTKIPNPRFQRLFQCLLHRALHPREPLPPIQQHIWNMLNPPAEVTTKSQIPLSKIKTLFPLIEAKKKDQVTAQEIFQDNHEDGPTAK

SEQ ID NO: 15 - RNA, 10 nt, Artificial Sequence
  Sm consensus site
aauuuuugga

SEQ ID NO: 16 - protein, 83 aa, Artificial Sequence
  Sm7 homoheptamer
GSVIDVSSQRVNVQRPLDALGNSLNSPVIIKLKGDREFRGVLKSFDLHMNLVLNDAEELEDGEVTRRLGTVLIRGDNIVYISP

SEQ ID NO: 17 - RNA, 23 nt, Artificial Sequence
  Ms2 phage operator stem loop
gcacaugaggaucacccaugugc

SEQ ID NO: 18 - protein, 117 aa, Artificial Sequence
  MS2 coat protein
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 19 - RNA, 26 nt, Artificial Sequence
  PP7 phage operator stem loop
auaaggaguuuauauggaaacccuua

SEQ ID NO: 20 - protein, 128 aa, Artificial Sequence
  PP7 coat protein (PCP)
MSKTIVLSVGEATRTLTEIQSTADRQIFEEKVGPLVGRLRLTASLRQNGAKTAYRVNLKLDQADVVDCSTSVCGELPKVRYTQVWSHDVTIVANSTEASRKSLYDLTKSLVATSQVEDLVVNLVPLGR

SEQ ID NO: 21 - RNA, 19 nt, Artificial Sequence
  SfMu Com stem loop
cugaaugccugcgagcauc

SEQ ID NO: 22 - protein, 62 aa, Artificial Sequence
  SfMu Com binding protein
MKSIRCKNCNKLLFKADSFDHIEIRCPRCKRHIIMLNACEHPTEKHCGKREKITHSDETVRY

SEQ ID NO: 23 - RNA, 15 nt, Artificial Sequence
  Box B aptamer
gcccugaagaagggc

SEQ ID NO: 24 - protein, 22 aa, Artificial Sequence
  Lambde N22plus protein
MNARTRRRERRAEKQAQWKAAN

SEQ ID NO: 25 - RNA, 16 nt, Artificial Sequence
  Csy4 binding motif
cugccguauaggcagc

SEQ ID NO: 26 - protein, 187 aa, Artificial Sequence
  Csy4[H29A]
MDHYLDIRLRPDPEFPPAQLMSVLFGKLAQALVAQGGDRIGVSFPDLDESRSRLGERLRIHASADDLRALLARPWLEGLRDHLQFGEPAVVPHPTPYRQVSRVQAKSNPERLRRRLMRRHDLSEEEARKRIPDTVARALDLPFVTLRSQSTGQHFRLFIRHGPLQVTAEEGGFTCYGLSKGGFVPWF

SEQ ID NO: 27 - RNA, 93 nt, Artificial Sequence
  Synthetic
guuuaagagcuaugcuggaaacagcauagcaaguuuaaauaaggcuaguccguuaucaacuugaaaaaguggcaccgagucggugcuuuuuuu

SEQ ID NO: 28 - RNA, 79 nt, Artificial Sequence
  Synthetic
ggaaccauucaaaacagcauagcaaguuaaaauaaggcuaguccguuaucaacuugaaaaaguggcaccgagucggugc

SEQ ID NO: 29 - RNA, 60 nt, Artificial Sequence
  Synthetic
uagcaaguuaaaauaaggcuaguccguuaucaacuugaaaaaguggcaccgagucggugc

SEQ ID NO: 30 - RNA, 64 nt, Artificial Sequence
  Synthetic
agcauagcaaguuaaaauaaggcuaguccguuaucaacuugaaaaaguggcaccgagucggugc

SEQ ID NO: 31 - RNA, 70 nt, Artificial Sequence
  Synthetic
caaaacagcauagcaaguuaaaauaaggcuaguccguuaucaacuugaaaaaguggcaccgagucggugc

SEQ ID NO: 32 - RNA, 45 nt, Artificial Sequence
  Synthetic
uagcaaguuaaaauaaggcuaguccguuaucaacuugaaaaagug

SEQ ID NO: 33 - RNA, 32 nt, Artificial Sequence
  Synthetic
uagcaaguuaaaauaaggcuaguccguuauca

SEQ ID NO: 34 - RNA, 26 nt, Artificial Sequence
  Synthetic
uagcaaguuaaaauaaggcuaguccg

SEQ ID NO: 35 - protein, 75 aa, Homo sapiens
MDAKSLTAWSRTLVTFKDVFVDFTREEWKLLDTAQQIVYRNVMLENYKNLVSLGYQLTKPDVILRLEKGEEPWLV

SEQ ID NO: 36 - protein, 311 aa, Homo sapiens
PSRLQMFFANNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACV

SEQ ID NO: 37 - DNA, 225 bp, Homo sapiens
atggacgcgaaatcacttacggcatggtcgagaacactggttacgttcaaggacgtgtttgtggactttacacgtgaggagtggaaattgctggatactgcgcaacaaattgtgtatcgaaatgtcatgcttgagaattacaagaacctcgtcagtctcggataccagttgacgaaaccggatgtgatccttaggctcgaaaagggggaagaaccttggctggta

SEQ ID NO: 38 - DNA, 933 bp, Homo sapiens
ccctcccggctccagatgttcttcgctaataaccacgaccaggaatttgaccctccaaaggtttacccacctgtcccagctgagaagaggaagcccatccgggtgctgtctctctttgatggaatcgctacagggctcctggtgctgaaggacttgggcattcaggtggaccgctacattgcctcggaggtgtgtgaggactccatcacggtgggcatggtgcggcaccaggggaagatcatgtacgtcggggacgtccgcagcgtcacacagaagcatatccaggagtggggcccattcgatctggtgattgggggcagtccctgcaatgacctctccatcgtcaaccctgctcgcaagggcctctacgagggcactggccggctcttctttgagttctaccgcctcctgcatgatgcgcggcccaaggagggagatgatcgccccttcttctggctctttgagaatgtggtggccatgggcgttagtgacaagagggacatctcgcgatttctcgagtccaaccctgtgatgattgatgccaaagaagtgtcagctgcacacagggcccgctacttctggggtaaccttcccggtatgaacaggccgttggcatccactgtgaatgataagctggagctgcaggagtgtctggagcatggcaggatagccaagttcagcaaagtgaggaccattactacgaggtcaaactccataaagcagggcaaagaccagcattttcctgtcttcatgaatgagaaagaggacatcttatggtgcactgaaatggaaagggtatttggtttcccagtccactatactgacgtctccaacatgagccgcttggcgaggcagagactgctgggccggtcatggagcgtgccagtcatccgccacctcttcgctccgctgaaggagtattttgcgtgtgtg

SEQ ID NO: 39 - protein, 1302 aa, Artificial Sequence
  Synthetic
MVDGKPIPNPLLGLDSTPKKKRKVNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIARGERNLIYVSVIDTCGNIVEQKSFNIVNGYDYQIKLKQQEGARQIARKEWKEIGKIKEIKEGYLSLVIHEISKMVIKYNAIIAMADLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDAAANGAYCIALKGLYEIKQITENWKEDGKFSRDKLKISNKDWFDFIQNKRYLKRPAATKKAGQAKKKK

SEQ ID NO: 40 - protein, 750 aa, Artificial Sequence
  Synthetic
MVDGSGPAAKRVKLDSGGIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVAVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAVKRPAATKKAGQAKKKK

SEQ ID NO: 41 - protein, 682 aa, Artificial Sequence
  Synthetic
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSAGGGGSGGGGSGGGGSGPKKKRKVAAAGSMSRRKQAKPQHFQSDPEVASLPRRDGDTEKGQPSRPTKSKDAHVCGRCCAEFFELSDLLLHKKNCTKNQLVLIVNENPASPPETFSPSPPPDNPDEQMNDTVNKTDQVDCSDLSEHNGLDREESMEVEAPVANKSGSGTSSGSHSSTAPSSSSSSSSSSGGGGSSSTGTSAITTSLPQLGDLTGSGGGSGGSGSMSAAGLLAPAPAQAGAPPAPEYYPEEDEELESAEDDERSCRGRESDEDTEDASETDLAKHDEEDYVEMKEQMYQDKLASLKRQLQQLQEGTLQEYQKRMKKLDQQYKERIRNAELFLQLETEQVERNYIKEKKAAVKEFEDKKVELKENLIAELEEKKKMIENEKLTMELTGDSMEVKPIMTRKLRRRPNDPVPIPDKRRKPAPAQLNYLLTDEQIMEDLRTLNKLKSPKRPASPSSPEHLPATPAESPAQRFEARIEDGKLYYDKRWYHKSQAIYLESKDNQKLSCVISSVGANEIWVRKTSDSTKMRIYLGQLQRGLFVIRRRSAA

SEQ ID NO: 42 - protein, 1942 aa, Artificial Sequence
  Synthetic
MSAAGLLAPAPAQAGAPPAPEYYPEEDEELESAEDDERSCRGRESDEDTEDASETDLAKHDEEDYVEMKEQMYQDKLASLKRQLQQLQEGTLQEYQKRMKKLDQQYKERIRNAELFLQLETEQVERNYIKEKKAAVKEFEDKKVELKENLIAELEEKKKMIENEKLTMELTGDSMEVKPIMTRKLRRRPNDPVPIPDKRRKPAPAQLNYLLTDEQIMEDLRTLNKLKSPKRPASPSSPEHLPATPAESPAQRFEARIEDGKLYYDKRWYHKSQAIYLESKDNQKLSCVISSVGANEIWVRKTSDSTKMRIYLGQLQRGLFVIRRRSAAGSGGGSGGSGSMSRRKQAKPQHFQSDPEVASLPRRDGDTEKGQPSRPTKSKDAHVCGRCCAEFFELSDLLLHKKNCTKNQLVLIVNENPASPPETFSPSPPPDNPDEQMNDTVNKTDQVDCSDLSEHNGLDREESMEVEAPVANKSGSGTSSGSHSSTAPSSSSSSSSSSGGGGSSSTGTSAITTSLPQLGDLTGSGGGSGGSGSMDYKDDDDKMAPKKKRKVGIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKK

SEQ ID NO: 43 - RNA, 100 nt, Artificial Sequence
  Synthetic
  misc_feature (1)..(2): 2′-O-methyl
  misc_feature (1)..(3): phosphorothioate linkage
  misc_feature (98)..(98): 2′-O-methyl
  misc_feature (98)..(100): phosphorothioate linkage
guaacgcgaacuacgcggguguuuuagagcuagaaauagcaaguuaaaauaaggcuaguccguuaucaacuugaaaaaguggcaccgagucggugcuuuu

SEQ ID NO: 44 - RNA, 100 nt, Artificial Sequence
  Synthetic
  misc_feature (1)..(2): 2′-O-methyl
  misc_feature (1)..(3): phosphorothioate linkage
  misc_feature (98)..(99): 2′-O-methyl
  misc_feature (98)..(100): phosphorothioate linkage
cucgcugagacuuccuggacguuuuagagcuagaaauagcaaguuaaaauaaggcuaguccguuaucaacuugaaaaaguggcaccgagucggugcuuuu

SEQ ID NO: 45 - RNA, 100 nt, Artificial Sequence
  Synthetic
  misc_feature (1)..(2): 2′-O-methyl
  misc_feature (1)..(3): phosphorothioate linkage
  misc_feature (98)..(99): 2′-O-methyl
  misc_feature (98)..(100): phosphorothioate linkage
ugaaggccuccugagcgcagguuuuagagcuagaaauagcaaguuaaaauaaggcuaguccguuaucaacuugaaaaaguggcaccgagucggugcuuuu

SEQ ID NO: 46 - RNA, 100 nt, Artificial Sequence
  Synthetic
  misc_feature (1)..(2): 2′-O-methyl
  misc_feature (1)..(3): phosphorothioate linkage
  misc_feature (98)..(99): 2′-O-methyl
  misc_feature (98)..(100): phosphorothioate linkage
ccacagccugucccccguccguuuuagagcuagaaauagcaaguuaaaauaaggcuaguccguuaucaacuugaaaaaguggcaccgagucggugcuuuu

SEQ ID NO: 47 - RNA, 100 nt, Artificial Sequence
  Synthetic
  misc_feature (1)..(3): phosphorothioate linkage
  misc_feature (1)..(2): 2′-O-methyl
  misc_feature (98)..(100): phosphorothioate linkage
  misc_feature (98)..(99): 2′-O-methyl
ucccuucuggguccagauauguuuuagagcuagaaauagcaaguuaaaauaaggcuaguccguuaucaacuugaaaaaguggcaccgagucggugcuuuu

SEQ ID NO: 48 - RNA, 100 nt, Artificial Sequence
  Synthetic
  misc_feature (1)..(2): 2′-O-methyl
  misc_feature (1)..(3): Phosphorothioate linkage
  misc_feature (98)..(99): 2′-O-methyl
  misc_feature (98)..(100): Phosphorothioate linkage
ggauuguugcgucccauaucguuuuagagcuagaaauagcaaguuaaaauaaggcuaguccguuaucaacuugaaaaaguggcaccgagucggugcuuuu

SEQ ID NO: 49 - RNA, 100 nt, Artificial Sequence
  Synthetic
  misc_feature (1)..(3): Phosphorothioate linkage
  misc_feature (1)..(2): 2′-O-methyl
  misc_feature (98)..(100): Phosphorothioate linkage
  misc_feature (98)..(99): 2′-O-methyl
gacuagagcucuccucagucguuuuagagcuagaaauagcaaguuaaaauaaggcuaguccguuaucaacuugaaaaaguggcaccgagucggugcuuuu

SEQ ID NO: 50 - RNA, 100 nt, Artificial Sequence
  Synthetic
  misc_feature (1)..(2): 2′-O-methyl
  misc_feature (1)..(3): Phosphorothioate linkage
  misc_feature (98)..(99): 2′-O-methyl
  misc_feature (98)..(100): Phosphorothioate linkage
cggagaggcgcagcauccacguuuuagagcuagaaauagcaaguuaaaauaaggcuaguccguuaucaacuugaaaaaguggcaccgagucggugcuuuu

SEQ ID NO: 51 - RNA, 100 nt, Artificial Sequence
  Synthetic
  misc_feature (1)..(3): Phosphorothioate linkage
  misc_feature (1)..(2): 2′-O-methyl
  misc_feature (98)..(100): Phosphorothioate linkage
  misc_feature (98)..(99): 2′-O-methyl
agaggcgcagcauccacaggguuuuagagcuagaaauagcaaguuaaaauaaggcuaguccguuaucaacuugaaaaaguggcaccgagucggugcuuuu

SEQ ID NO: 52 - RNA, 100 nt, Artificial Sequence
  Synthetic
  misc_feature (1)..(2): 2′-O-methyl
  misc_feature (1)..(3): Phosphorothioate linkage
  misc_feature (98)..(99): 2′-O-methyl
  misc_feature (98)..(100): Phosphorothioate linkage
ggaccccgcgaugagggcggguuuuagagcuagaaauagcaaguuaaaauaaggcuaguccguuaucaacuugaaaaaguggcaccgagucggugcuuuu

SEQ ID NO: 53 - RNA, 100 nt, Artificial Sequence
  Synethetic
  misc_feature (1)..(3): Phosphorothioate linkage
  misc_feature (1)..(2): 2′-O-methyl
  misc_feature (98)..(100): Phosphorothioate linkage
  misc_feature (98)..(99): 2′-O-methyl
aacugggccugaaaggguacguuuuagagcuagaaauagcaaguuaaaauaaggcuaguccguuaucaacuugaaaaaguggcaccgagucggugcuuuu

SEQ ID NO: 54 - RNA, 100 nt, Artificial Sequence
  Synethetic
  misc_feature (1)..(2): 2′-O-methyl
  misc_feature (1)..(3): Phosphorothioate linkage
  misc_feature (98)..(99): 2′-O-methyl
  misc_feature (98)..(100): Phosphorothioate linkage
gcgccgccggcccagcuauaguuuuagagcuagaaauagcaaguuaaaauaaggcuaguccguuaucaacuugaaaaaguggcaccgagucggugcuuuu

SEQ ID NO: 55 - RNA, 100 nt, Artificial Sequence
  Synethetic
  misc_feature (1)..(3): Phosphorothioate linkage
  misc_feature (1)..(2): 2′-O-methyl
  misc_feature (98)..(100): Phosphorothioate linkage
  misc_feature (98)..(99): 2′-O-methyl
ucccugccacacgcaaacacguuuuagagcuagaaauagcaaguuaaaauaaggcuaguccguuaucaacuugaaaaaguggcaccgagucggugcuuuu

SEQ ID NO: 56 - RNA, 100 nt, Artificial Sequence
  Synethetic
  misc_feature (1)..(2): 2′-O-methyl
  misc_feature (1)..(3): Phosphorothioate linkage
  misc_feature (98)..(99): 2′-O-methyl
  misc_feature (98)..(100): Phosphorothioate linkage
agggggcggauacugacccgguuuuagagcuagaaauagcaaguuaaaauaaggcuaguccguuaucaacuugaaaaaguggcaccgagucggugcuuuu

SEQ ID NO: 57 - RNA, 100 nt, Artificial Sequence
  Synethetic
  misc_feature (1)..(3): Phosphorothioate linkage
  misc_feature (1)..(2): 2′-O-methyl
  misc_feature (98)..(100): Phosphorothioate linkage
  misc_feature (98)..(99): 2′-O-methyl
auacugacccgaggacgccgguuuuagagcuagaaauagcaaguuaaaauaaggcuaguccguuaucaacuugaaaaaguggcaccgagucggugcuuuu

SEQ ID NO: 58 - RNA, 100 nt, Artificial Sequence
  Synethetic
  misc_feature (1)..(2): 2′-O-methyl
  misc_feature (1)..(3): Phosphorothioate linkage
  misc_feature (98)..(99): 2′-O-methyl
  misc_feature (98)..(100): Phosphorothioate linkage
ggugguggcugaguccguggguuuuagagcuagaaauagcaaguuaaaauaaggcuaguccguuaucaacuugaaaaaguggcaccgagucggugcuuuu

SEQ ID NO: 59 - RNA, 100 nt, Artificial Sequence
  Synethetic
  misc_feature (1)..(3): Phosphorothioate linkage
  misc_feature (1)..(2): 2′-O-methyl
  misc_feature (98)..(100): Phosphorothioate linkage
  misc_feature (98)..(99): 2′-O-methyl
ccgcuaggcgcaccgaccggguuuuagagcuagaaauagcaaguuaaaauaaggcuaguccguuaucaacuugaaaaaguggcaccgagucggugcuuuu

SEQ ID NO: 60 - RNA, 100 nt, Artificial Sequence
  Synethetic
  misc_feature (1)..(2): 2′-O-methyl
  misc_feature (1)..(3): Phosphorothioate linkage
  misc_feature (98)..(99): 2′-O-methyl
  misc_feature (98)..(100): Phosphorothioate linkage
gauccgcuaggcgcaccgacguuuuagagcuagaaauagcaaguuaaaauaaggcuaguccguuaucaacuugaaaaaguggcaccgagucggugcuuuu

SEQ ID NO: 61 - RNA, 100 nt, Artificial Sequence
  Synethetic
  misc_feature (1)..(3): Phosphorothioate linkage
  misc_feature (1)..(2): 2′-O-methyl
  misc_feature (98)..(100): Phosphorothioate linkage
  misc_feature (98)..(99): 2′-O-methyl
gcuggcgcgacggcucgacuguuuuagagcuagaaauagcaaguuaaaauaaggcuaguccguuaucaacuugaaaaaguggcaccgagucggugcuuuu

SEQ ID NO: 62 - RNA, 100 nt, Artificial Sequence
  Synethetic
  misc_feature (1)..(2): 2′-O-methyl
  misc_feature (1)..(3): Phosphorothioate linkage
  misc_feature (98)..(99): 2′-O-methyl
  misc_feature (98)..(100): Phosphorothioate linkage
ugcgcggccgaggagcgaaaguuuuagagcuagaaauagcaaguuaaaauaaggcuaguccguuaucaacuugaaaaaguggcaccgagucggugcuuuu

SEQ ID NO: 63 - RNA, 100 nt, Artificial Sequence
  Synethetic
  misc_feature (1)..(3): Phosphorothioate linkage
  misc_feature (1)..(2): 2′-O-methyl
  misc_feature (98)..(100): Phosphorothioate linkage
  misc_feature (98)..(99): 2′-O-methyl
gcggccgaggagcgaaagggguuuuagagcuagaaauagcaaguuaaaauaaggcuaguccguuaucaacuugaaaaaguggcaccgagucggugcuuuu

SEQ ID NO: 64 - RNA, 100 nt, Artificial Sequence
  Synethetic
  misc_feature (1)..(2): 2′-O-methyl
  misc_feature (1)..(3): Phosphorothioate linkage
  misc_feature (98)..(99): 2′-O-methyl
  misc_feature (98)..(100): Phosphorothioate linkage
ggagcagcagugguaucuguguuuuagagcuagaaauagcaaguuaaaauaaggcuaguccguuaucaacuugaaaaaguggcaccgagucggugcuuuu

SEQ ID NO: 65 - RNA, 100 nt, Artificial Sequence
  Synethetic
  misc_feature (1)..(3): Phosphorothioate linkage
  misc_feature (1)..(2): 2′-O-methyl
  misc_feature (98)..(100): Phosphorothioate linkage
  misc_feature (98)..(99): 2′-O-methyl
aagcuugaaagacuugguaaguuuuagagcuagaaauagcaaguuaaaauaaggcuaguccguuaucaacuugaaaaaguggcaccgagucggugcuuuu

SEQ ID NO: 66 - RNA, 100 nt, Artificial Sequence
  Synthetic
  misc_feature (1)..(2): 2′-O-methyl
  misc_feature (1)..(3): Phosphorothioate linkage
  misc_feature (98)..(99): 2′-O-methyl
  misc_feature (98)..(100): Phosphorothioate linkage
uugaggaguaagagcugccgguuuuagagcuagaaauagcaaguuaaaauaaggcuaguccguuaucaacuugaaaaaguggcaccgagucggugcuuuu

SEQ ID NO: 67 - RNA, 100 nt, Artificial Sequence
  Synethetic
  misc_feature (1)..(3): Phosphorothioate linkage
  misc_feature (1)..(2): 2′-O-methyl
  misc_feature (98)..(100): Phosphorothioate linkage
  misc_feature (98)..(99): 2′-O-methyl
uggcgacggguuugguaaguguuuuagagcuagaaauagcaaguuaaaauaaggcuaguccguuaucaacuugaaaaaguggcaccgagucggugcuuuu

SEQ ID NO: 68 - RNA, 100 nt, Artificial Sequence
  Synethetic
  misc_feature (1)..(2): 2′-O-methyl
  misc_feature (1)..(3): Phosphorothioate linkage
  misc_feature (98)..(99): 2′-O-methyl
  misc_feature (98)..(100): Phosphorothioate linkage
cacgagcgcgagauagcgucguuuuagagcuagaaauagcaaguuaaaauaaggcuaguccguuaucaacuugaaaaaguggcaccgagucggugcuuuu

SEQ ID NO: 69 - RNA, 100 nt, Artificial Sequence
  Synethetic
  misc_feature (1)..(3): Phosphorothioate linkage
  misc_feature (1)..(2): 2′-O-methyl
  misc_feature (98)..(100): Phosphorothioate linkage
  misc_feature (98)..(99): 2′-O-methyl
agcgucgggccgcacgaugaguuuuagagcuagaaauagcaaguuaaaauaaggcuaguccguuaucaacuugaaaaaguggcaccgagucggugcuuuu

SEQ ID NO: 70 - RNA, 100 nt, Artificial Sequence
  Synethetic
  misc_feature (1)..(2): 2′-O-methyl
  misc_feature (1)..(3): Phosphorothioate linkage
  misc_feature (98)..(99): 2′-O-methyl
  misc_feature (98)..(100): Phosphorothioate linkage
gcucgugugcaggcccggcuguuuuagagcuagaaauagcaaguuaaaauaaggcuaguccguuaucaacuugaaaaaguggcaccgagucggugcuuuu

SEQ ID NO: 71 - RNA, 100 nt, Artificial Sequence
  Synethetic
  misc_feature (1)..(3): Phosphorothioate linkage
  misc_feature (1)..(2): 2′-O-methyl
  misc_feature (98)..(100): Phosphorothioate linkage
  misc_feature (98)..(99): 2′-O-methyl
ggcggccgcggcggugaacgguuuuagagcuagaaauagcaaguuaaaauaaggcuaguccguuaucaacuugaaaaaguggcaccgagucggugcuuuu

SEQ ID NO: 72 - RNA, 100 nt, Artificial Sequence
  Synethetic
  misc_feature (1)..(2): 2′-O-methyl
  misc_feature (1)..(3): Phosphorothioate linkage
  misc_feature (98)..(99): 2′-O-methyl
  misc_feature (98)..(100): Phosphorothioate linkage
ugccgcaucacgcaagauggguuuuagagcuagaaauagcaaguuaaaauaaggcuaguccguuaucaacuugaaaaaguggcaccgagucggugcuuuu

SEQ ID NO: 73 - RNA, 100 nt, Artificial Sequence
  Synethetic
  misc_feature (1)..(3): Phosphorothioate linkage
  misc_feature (1)..(2): 2′-O-methyl
  misc_feature (98)..(100): Phosphorothioate linkage
  misc_feature (98)..(99): 2′-O-methyl
cggcgcugccguaaaucaggguuuuagagcuagaaauagcaaguuaaaauaaggcuaguccguuaucaacuugaaaaaguggcaccgagucggugcuuuu

SEQ ID NO: 74 - RNA, 100 nt, Artificial Sequence
  Synethetic
  misc_feature (1)..(2): 2′-O-methyl
  misc_feature (1)..(3): Phosphorothioate linkage
  misc_feature (98)..(99): 2′-O-methyl
  misc_feature (98)..(100): Phosphorothioate linkage
acggugugagagcgguaagaguuuuagagcuagaaauagcaaguuaaaauaaggcuaguccguuaucaacuugaaaaaguggcaccgagucggugcuuuu

SEQ ID NO: 75 - RNA, 100 nt, Artificial Sequence
  Synethetic
  misc_feature (1)..(3): Phosphorothioate linkage
  misc_feature (1)..(2): 2′-O-methyl
  misc_feature (98)..(100): Phosphorothioate linkage
  misc_feature (98)..(99): 2′-O-methyl
ggccggggacggugugagagguuuuagagcuagaaauagcaaguuaaaauaaggcuaguccguuaucaacuugaaaaaguggcaccgagucggugcuuuu

SEQ ID NO: 76 - RNA, 100 nt, Artificial Sequence
  Synethetic
  misc_feature (1)..(2): 2′-O-methyl
  misc_feature (1)..(3): Phosphorothioate linkage
  misc_feature (98)..(99): 2′-O-methyl
  misc_feature (98)..(100): Phosphorothioate linkage
gugugagagcgguaagauggguuuuagagcuagaaauagcaaguuaaaauaaggcuaguccguuaucaacuugaaaaaguggcaccgagucggugcuuuu

SEQ ID NO: 77 - RNA, 100 nt, Artificial Sequence
  Synethetic
  misc_feature (1)..(3): Phosphorothioate linkage
  misc_feature (1)..(2): 2′-O-methyl
  misc_feature (98)..(100): Phosphorothioate linkage
  misc_feature (98)..(99): 2′-O-methyl
ggcuccggaguuuauccuccguuuuagagcuagaaauagcaaguuaaaauaaggcuaguccguuaucaacuugaaaaaguggcaccgagucggugcuuuu

SEQ ID NO: 78 - RNA, 100 nt, Artificial Sequence
  Synethetic
  misc_feature (1)..(2): 2′-O-methyl
  misc_feature (1)..(3): Phosphorothioate linkage
  misc_feature (98)..(99): 2′-O-methyl
  misc_feature (98)..(100): Phosphorothioate linkage
gagaaggagcaagaagugucguuuuagagcuagaaauagcaaguuaaaauaaggcuaguccguuaucaacuugaaaaaguggcaccgagucggugcuuuu

SEQ ID NO: 79 - RNA, 100 nt, Artificial Sequence
  Synethetic
  misc_feature (1)..(3): Phosphorothioate linkage
  misc_feature (1)..(2): 2′-O-methyl
  misc_feature (98)..(100): Phosphorothioate linkage
  misc_feature (98)..(99): 2′-O-methyl
ccgcgagguggcaguggcagguuuuagagcuagaaauagcaaguuaaaauaaggcuaguccguuaucaacuugaaaaaguggcaccgagucggugcuuuu

SEQ ID NO: 80 - RNA, 100 nt, Artificial Sequence
  Synethetic
  misc_feature (1)..(2): 2′-O-methyl
  misc_feature (1)..(3): Phosphorothioate linkage
  misc_feature (98)..(99): 2′-O-methyl
  misc_feature (98)..(100): Phosphorothioate linkage
cugaauuccgcgggagagaaguuuuagagcuagaaauagcaaguuaaaauaaggcuaguccguuaucaacuugaaaaaguggcaccgagucggugcuuuu

SEQ ID NO: 81 - RNA, 100 nt, Artificial Sequence
  Synethetic
  misc_feature (1)..(3): Phosphorothioate linkage
  misc_feature (1)..(2): 2′-O-methyl
  misc_feature (98)..(100): Phosphorothioate linkage
  misc_feature (98)..(99): 2′-O-methyl
uccgugaaaagaaaacaacaguuuuagagcuagaaauagcaaguuaaaauaaggcuaguccguuaucaacuugaaaaaguggcaccgagucggugcuuuu

SEQ ID NO: 82 - RNA, 100 nt, Artificial Sequence
  Synethetic
  misc_feature (1)..(2): 2′-O-methyl
  misc_feature (1)..(3): Phosphorothioate linkage
  misc_feature (98)..(99): 2′-O-methyl
  misc_feature (98)..(100): Phosphorothioate linkage
ggccguaaaccugaauuccgguuuuagagcuagaaauagcaaguuaaaauaaggcuaguccguuaucaacuugaaaaaguggcaccgagucggugcuuuu

SEQ ID NO: 83 - RNA, 100 nt, Artificial Sequence
  Synethetic
  misc_feature (1)..(3): Phosphorothioate linkage
  misc_feature (1)..(2): 2′-O-methyl
  misc_feature (98)..(100): Phosphorothioate linkage
  misc_feature (98)..(99): 2′-O-methyl
ggguaaagauggcggagcgcguuuuagagcuagaaauagcaaguuaaaauaaggcuaguccguuaucaacuugaaaaaguggcaccgagucggugcuuuu

SEQ ID NO: 84 - RNA, 100 nt, Artificial Sequence
  Synethetic
  misc_feature (1)..(2): 2′-O-methyl
  misc_feature (1)..(3): Phosphorothioate linkage
  misc_feature (98)..(99): 2′-O-methyl
  misc_feature (98)..(100): Phosphorothioate linkage
gcuuuucgcugacuacauucguuuuagagcuagaaauagcaaguuaaaauaaggcuaguccguuaucaacuugaaaaaguggcaccgagucggugcuuuu

SEQ ID NO: 85 - RNA, 100 nt, Artificial Sequence
  Synethetic
  misc_feature (1)..(2): 2′-O-methyl
  misc_feature (1)..(3): Phosphorothioate linkage
  misc_feature (98)..(99): 2′-O-methyl
  misc_feature (98)..(100): Phosphorothioate linkage
gacuacgcugaagaccucgaguuuuagagcuagaaauagcaaguuaaaauaaggcuaguccguuaucaacuugaaaaaguggcaccgagucggugcuuuu

SEQ ID NO: 86 - RNA, 100 nt, Artificial Sequence
  Synethetic
  misc_feature (1)..(2): 2′-O-methyl
  misc_feature (1)..(3): Phosphorothioate linkage
  misc_feature (98)..(99): 2′-O-methyl
  misc_feature (98)..(100): Phosphorothioate linkage
cccggacucggacgcgugguguuuuagagcuagaaauagcaaguuaaaauaaggcuaguccguuaucaacuugaaaaaguggcaccgagucggugcuuuu

SEQ ID NO: 87 - RNA, 100 nt, Artificial Sequence
  Synethetic
  misc_feature (1)..(2): 2′-O-methyl
  misc_feature (1)..(3): Phosphorothioate linkage
  misc_feature (98)..(99): 2′-O-methyl
  misc_feature (98)..(100): Phosphorothioate linkage
gcggcccggagccuacgaggguuuuagagcuagaaauagcaaguuaaaauaaggcuaguccguuaucaacuugaaaaaguggcaccgagucggugcuuuu

SEQ ID NO: 88 - RNA, 100 nt, Artificial Sequence
  Synethetic
  misc_feature (1)..(2): 2′-O-methyl
  misc_feature (1)..(3): Phosphorothioate linkage
  misc_feature (98)..(99): 2′-O-methyl
  misc_feature (98)..(100): Phosphorothioate linkage
agggcccggacucggacgcgguuuuagagcuagaaauagcaaguuaaaauaaggcuaguccguuaucaacuugaaaaaguggcaccgagucggugcuuuu

SEQ ID NO: 89 - RNA, 100 nt, Artificial Sequence
  Synethetic
  misc_feature (1)..(2): 2′-O-methyl
  misc_feature (1)..(3): Phosphorothioate linkage
  misc_feature (98)..(99): 2′-O-methyl
  misc_feature (98)..(100): Phosphorothioate linkage
aaccaacccccaggcgguggguuuuagagcuagaaauagcaaguuaaaauaaggcuaguccguuaucaacuugaaaaaguggcaccgagucggugcuuuu

SEQ ID NO: 90 - RNA, 100 nt, Artificial Sequence
  Synethetic
  misc_feature (1)..(2): 2′-O-methyl
  misc_feature (1)..(3): Phosphorothioate linkage
  misc_feature (98)..(99): 2′-O-methyl
  misc_feature (98)..(100): Phosphorothioate linkage
cucaaccaacccccaggcggguuuuagagcuagaaauagcaaguuaaaauaaggcuaguccguuaucaacuugaaaaaguggcaccgagucggugcuuuu

SEQ ID NO: 91 - RNA, 100 nt, Artificial Sequence
  Synethetic
  misc_feature (1)..(2): 2′-O-methyl
  misc_feature (1)..(3): Phosphorothioate linkage
  misc_feature (98)..(99): 2′-O-methyl
  misc_feature (98)..(100): Phosphorothioate linkage
ccucgcagagcucggagacgguuuuagagcuagaaauagcaaguuaaaauaaggcuaguccguuaucaacuugaaaaaguggcaccgagucggugcuuuu

SEQ ID NO: 92 - RNA, 100 nt, Artificial Sequence
  Synethetic
  misc_feature (1)..(3): Phosphorothioate linkage
  misc_feature (1)..(2): 2′-O-methyl
  misc_feature (98)..(100): Phosphorothioate linkage
  misc_feature (98)..(99): 2′-O-methyl
gcagccauagggagccgcacguuuuagagcuagaaauagcaaguuaaaauaaggcuaguccguuaucaacuugaaaaaguggcaccgagucggugcuuuu

SEQ ID NO: 93 - RNA, 100 nt, Artificial Sequence
  Synethetic
  misc_feature (1)..(2): 2′-O-methyl
  misc_feature (1)..(3): Phosphorothioate linkage
  misc_feature (98)..(99): 2′-O-methyl
  misc_feature (98)..(100): Phosphorothioate linkage
ggauggcggccccuaaccggguuuuagagcuagaaauagcaaguuaaaauaaggcuaguccguuaucaacuugaaaaaguggcaccgagucggugcuuuu

SEQ ID NO: 94 - RNA, 100 nt, Artificial Sequence
  Synethetic
  misc_feature (1)..(2): 2′-O-methyl
  misc_feature (1)..(3): Phosphorothioate linkage
  misc_feature (98)..(99): 2′-O-methyl
  misc_feature (98)..(100): Phosphorothioate linkage
cagagcgucgggauaucgggguuuuagagcuagaaauagcaaguuaaaauaaggcuaguccguuaucaacuugaaaaaguggcaccgagucggugcuuuu

SEQ ID NO: 95 - RNA, 100 nt, Artificial Sequence
  Synethetic
  misc_feature (1)..(2): 2′-O-methyl
  misc_feature (1)..(3): Phosphorothioate linkage
  misc_feature (98)..(99): 2′-O-methyl
  misc_feature (98)..(100): Phosphorothioate linkage
auagucgcacagcaacccggguuuuagagcuagaaauagcaaguuaaaauaaggcuaguccguuaucaacuugaaaaaguggcaccgagucggugcuuuu

SEQ ID NO: 96 - RNA, 100 nt, Artificial Sequence
  Synethetic
  misc_feature (1)..(2): 2′-O-methyl
  misc_feature (1)..(3): Phosphorothioate linkage
  misc_feature (98)..(99): 2′-O-methyl
  misc_feature (98)..(100): Phosphorothioate linkage
agaauagucgcacagcaaccguuuuagagcuagaaauagcaaguuaaaauaaggcuaguccguuaucaacuugaaaaaguggcaccgagucggugcuuuu

SEQ ID NO: 97 - RNA, 100 nt, Artificial Sequence
  Synethetic
  misc_feature (1)..(2): 2′-O-methyl
  misc_feature (1)..(3): Phosphorothioate linkage
  misc_feature (98)..(99): 2′-O-methyl
  misc_feature (98)..(100): Phosphorothioate linkage
gguuccggcggugacacggaguuuuagagcuagaaauagcaaguuaaaauaaggcuaguccguuaucaacuugaaaaaguggcaccgagucggugcuuuu

SEQ ID NO: 98 - RNA, 100 nt, Artificial Sequence
  Synethetic
  misc_feature (1)..(2): 2′-O-methyl
  misc_feature (1)..(3): Phosphorothioate linkage
  misc_feature (98)..(99): 2′-O-methyl
  misc_feature (98)..(100): Phosphorothioate linkage
ccggaaguagagucacggagguuuuagagcuagaaauagcaaguuaaaauaaggcuaguccguuaucaacuugaaaaaguggcaccgagucggugcuuuu

SEQ ID NO: 99 - RNA, 100 nt, Artificial Sequence
  Synethetic
  misc_feature (1)..(3): Phosphorothioate linkage
  misc_feature (1)..(2): 2′-O-methyl
  misc_feature (98)..(100): Phosphorothioate linkage
  misc_feature (98)..(99): 2′-O-methyl
agagguaggauccggaagugguuuuagagcuagaaauagcaaguuaaaauaaggcuaguccguuaucaacuugaaaaaguggcaccgagucggugcuuuu

SEQ ID NO: 100 - RNA, 100 nt, Artificial Sequence
  Synethetic
  misc_feature (1)..(2): 2′-O-methyl
  misc_feature (1)..(3): Phosphorothioate linkage
  misc_feature (98)..(99): 2′-O-methyl
  misc_feature (98)..(100): Phosphorothioate linkage
agauaccggaaguagagucaguuuuagagcuagaaauagcaaguuaaaauaaggcuaguccguuaucaacuugaaaaaguggcaccgagucggugcuuuu

SEQ ID NO: 101 - RNA, 100 nt, Artificial Sequence
  Synethetic
  misc_feature (1)..(3): Phosphorothioate linkage
  misc_feature (1)..(2): 2′-O-methyl
  misc_feature (98)..(100): Phosphorothioate linkage
  misc_feature (98)..(99): 2′-O-methyl
uuaccucaaggaccauggcuguuuuagagcuagaaauagcaaguuaaaauaaggcuaguccguuaucaacuugaaaaaguggcaccgagucggugcuuuu

SEQ ID NO: 102 - RNA, 104 nt, Artificial Sequence
  Synethetic
mgmggcagcagcacuuaccucaguuuuagagcuagaaauagcaaguuaaaauaaggcuaguccguuaucaacuugaaaaaguggcaccgagucggugcumumuu

SEQ ID NO: 103 - RNA, 100 nt, Artificial Sequence
  Synethetic
  misc_feature (1)..(2): 2′-O-methyl
  misc_feature (1)..(3): Phosphorothioate linkage
  misc_feature (98)..(99): 2′-O-methyl
  misc_feature (98)..(100): Phosphorothioate linkage
ccacaagcaccaaagcagagguuuuagagcuagaaauagcaaguuaaaauaaggcuaguccguuaucaacuugaaaaaguggcaccgagucggugcuuuu

SEQ ID NO: 104 - RNA, 100 nt, Artificial Sequence
  Synethetic
  misc_feature (1)..(3): Phosphorothioate linkage
  misc_feature (1)..(2): 2′-O-methyl
  misc_feature (98)..(100): Phosphorothioate linkage
  misc_feature (98)..(99): 2′-O-methyl
gccgauggugaagugguaagguuuuagagcuagaaauagcaaguuaaaauaaggcuaguccguuaucaacuugaaaaaguggcaccgagucggugcuuuu

SEQ ID NO: 105 - RNA, 100 nt, Artificial Sequence
  Synethetic
  misc_feature (1)..(2): 2′-O-methyl
  misc_feature (1)..(3): Phosphorothioate linkage
  misc_feature (98)..(99): 2′-O-methyl
  misc_feature (98)..(100): Phosphorothioate linkage
guguauuuugaccuacgaauguuuuagagcuagaaauagcaaguuaaaauaaggcuaguccguuaucaacuugaaaaaguggcaccgagucggugcuuuu

SEQ ID NO: 106 - RNA, 94 nt, Artificial Sequence
  Synethetic
  misc_feature (1)..(3): Phosphorothioate linkage
  misc_feature (1)..(2): 2′-O-methyl
  misc_feature (92)..(94): Phosphorothioate linkage
  misc_feature (92)..(93): 2′-O-methyl
cgaacuacgcggguguuuuagagcuagaaauagcaaguuaaaauaaggcuaguccguuaucaacuugaaaaaguggcaccgagucggugcuuuu

SEQ ID NO: 107 - RNA, 94 nt, Artificial Sequence
  Synethetic
  misc_feature (1)..(2): 2′-O-methyl
  misc_feature (1)..(3): Phosphorothioate linkage
  misc_feature (92)..(93): 2′-O-methyl
  misc_feature (92)..(94): Phosphorothioate linkage
gcacagcaacccggguuuuagagcuagaaauagcaaguuaaaauaaggcuaguccguuaucaacuugaaaaaguggcaccgagucggugcuuuu

SEQ ID NO: 108 - RNA, 94 nt, Artificial Sequence
  Synethetic
  misc_feature (1)..(3): Phosphorothioate linkage
  misc_feature (1)..(2): 2′-O-methyl
  misc_feature (92)..(94): Phosphorothioate linkage
  misc_feature (92)..(93): 2′-O-methyl
gucgcacagcaaccguuuuagagcuagaaauagcaaguuaaaauaaggcuaguccguuaucaacuugaaaaaguggcaccgagucggugcuuuu

SEQ ID NO: 109 - RNA, 94 nt, Artificial Sequence
  Synethetic
  misc_feature (1)..(2): 2′-O-methyl
  misc_feature (1)..(3): Phosphorothioate linkage
  misc_feature (92)..(93): 2′-O-methyl
  misc_feature (92)..(94): Phosphorothioate linkage
ggcggugacacggaguuuuagagcuagaaauagcaaguuaaaauaaggcuaguccguuaucaacuugaaaaaguggcaccgagucggugcuuuu

SEQ ID NO: 110 - RNA, 94 nt, Artificial Sequence
  Synethetic
  misc_feature (1)..(3): Phosphorothioate linkage
  misc_feature (1)..(2): 2′-O-methyl
  misc_feature (92)..(94): Phosphorothioate linkage
  misc_feature (92)..(93): 2′-O-methyl
uccgcgggagagaaguuuuagagcuagaaauagcaaguuaaaauaaggcuaguccguuaucaacuugaaaaaguggcaccgagucggugcuuuu

SEQ ID NO: 111 - RNA, 94 nt, Artificial Sequence
  Synethetic
  misc_feature (1)..(2): 2′-O-methyl
  misc_feature (1)..(3): Phosphorothioate linkage
  misc_feature (92)..(93): 2′-O-methyl
  misc_feature (92)..(94): Phosphorothioate linkage
aaaagaaaacaacaguuuuagagcuagaaauagcaaguuaaaauaaggcuaguccguuaucaacuugaaaaaguggcaccgagucggugcuuuu

SEQ ID NO: 112 - RNA, 94 nt, Artificial Sequence
  Synethetic
  misc_feature (1)..(3): Phosphorothioate linkage
  misc_feature (1)..(3): 2′-O-methyl
  misc_feature (92)..(94): Phosphorothioate linkage
  misc_feature (92)..(94): 2′-O-methyl
aaaccugaauuccgguuuuagagcuagaaauagcaaguuaaaauaaggcuaguccguuaucaacuugaaaaaguggcaccgagucggugcuuuu

SEQ ID NO: 113 - RNA, 94 nt, Artificial Sequence
  Synethetic
  misc_feature (1)..(2): 2′-O-methyl
  misc_feature (1)..(3): Phosphorothioate linkage
  misc_feature (92)..(93): 2′-O-methyl
  misc_feature (92)..(94): Phosphorothioate linkage
cggauacugacccgguuuuagagcuagaaauagcaaguuaaaauaaggcuaguccguuaucaacuugaaaaaguggcaccgagucggugcuuuu

SEQ ID NO: 114 - RNA, 94 nt, Artificial Sequence
  Synethetic
  misc_feature (1)..(3): Phosphorothioate linkage
  misc_feature (1)..(2): 2′-O-methyl
  misc_feature (92)..(94): Phosphorothioate linkage
  misc_feature (92)..(93): 2′-O-methyl
acccgaggacgccgguuuuagagcuagaaauagcaaguuaaaauaaggcuaguccguuaucaacuugaaaaaguggcaccgagucggugcuuuu

SEQ ID NO: 115 - RNA, 94 nt, Artificial Sequence
  Synethetic
  misc_feature (1)..(2): 2′-O-methyl
  misc_feature (1)..(3): Phosphorothioate linkage
  misc_feature (92)..(93): 2′-O-methyl
  misc_feature (92)..(94): Phosphorothioate linkage
ggcugaguccguggguuuuagagcuagaaauagcaaguuaaaauaaggcuaguccguuaucaacuugaaaaaguggcaccgagucggugcuuuu

SEQ ID NO: 116 - RNA, 94 nt, Artificial Sequence
  Synethetic
  misc_feature (1)..(3): Phosphorothioate linkage
  misc_feature (1)..(2): 2′-O-methyl
  misc_feature (92)..(94): Phosphorothioate linkage
  misc_feature (92)..(93): 2′-O-methyl
guagagucacggagguuuuagagcuagaaauagcaaguuaaaauaaggcuaguccguuaucaacuugaaaaaguggcaccgagucggugcuuuu

SEQ ID NO: 117 - RNA, 94 nt, Artificial Sequence
  Synethetic
  misc_feature (1)..(2): 2′-O-methyl
  misc_feature (1)..(3): Phosphorothioate linkage
  misc_feature (92)..(93): 2′-O-methyl
  misc_feature (92)..(94): Phosphorothioate linkage
aggauccggaagugguuuuagagcuagaaauagcaaguuaaaauaaggcuaguccguuaucaacuugaaaaaguggcaccgagucggugcuuuu

SEQ ID NO: 118 - RNA, 94 nt, Artificial Sequence
  Synethetic
  misc_feature (1)..(3): Phosphorothioate linkage
  misc_feature (1)..(2): 2′-O-methyl
  misc_feature (92)..(94): Phosphorothioate linkage
  misc_feature (92)..(93): 2′-O-methyl
cggaaguagagucaguuuuagagcuagaaauagcaaguuaaaauaaggcuaguccguuaucaacuugaaaaaguggcaccgagucggugcuuuu

SEQ ID NO: 119 - RNA, 94 nt, Artificial Sequence
  Synethetic
  misc_feature (1)..(2): 2′-O-methyl
  misc_feature (1)..(3): Phosphorothioate linkage
  misc_feature (92)..(93): 2′-O-methyl
  misc_feature (92)..(94): Phosphorothioate linkage
ggugaagugguaagguuuuagagcuagaaauagcaaguuaaaauaaggcuaguccguuaucaacuugaaaaaguggcaccgagucggugcuuuu

SEQ ID NO: 120 - RNA, 94 nt, Artificial Sequence
  Synethetic
  misc_feature (1)..(3): Phosphorothioate linkage
  misc_feature (1)..(2): 2′-O-methyl
  misc_feature (92)..(94): Phosphorothioate linkage
  misc_feature (92)..(93): 2′-O-methyl
uuugaccuacgaauguuuuagagcuagaaauagcaaguuaaaauaaggcuaguccguuaucaacuugaaaaaguggcaccgagucggugcuuuu

SEQ ID NO: 121 - RNA, 100 nt, Artificial Sequence
  Synethetic
  misc_feature (1)..(1): G-LNA
  misc_feature (2)..(2): 5′-MethylC-LNA
  misc_feature (98)..(99): 2′-O-methyl
  misc_feature (98)..(100): Phosphorothioate linkage
gccgccggcccagcuauaguuuuagagcuagaaauagcaaguuaaaauaaggcuaguccguuaucaacuugaaaaaguggcaccgagucggugcumumuu

SEQ ID NO: 122 - DNA, 100 bp, Artificial Sequence
  Synethetic
  misc_feature (1)..(2): 2′-O-methyl
  misc_feature (1)..(3): Phosphorothioate linkage
  misc_feature (98)..(99): T-LNA
gcgccgccggcccagcuauaguuuuagagcuagaaauagcaaguuaaaauaaggcuaguccguuaucaacuugaaaaaguggcaccgagucggugcuttu

SEQ ID NO: 123 - DNA, 100 bp, Artificial Sequence; identical to SEQ ID NO: 122
  Synethetic
  misc_feature (1)..(1): G-LNA
  misc_feature (2)..(2): 5-methyl C-LNA
  misc_feature (98)..(99): T-LNA

SEQ ID NO: 124 - DNA, 100 bp, Artificial Sequence
  Synethetic
  misc_feature (1)..(1): G-LNA
  misc_feature (2)..(2): T-LNA
  misc_feature (98)..(99): 2′-O-methyl
  misc_feature (98)..(100): Phosphorothioate linkage
gtgugagagcgguaagauggguuuuagagcuagaaauagcaaguuaaaauaaggcuaguccguuaucaacuugaaaaaguggcaccgagucggugcuuuu

SEQ ID NO: 125 - DNA, 100 bp, Artificial Sequence
  Synethetic
  misc_feature (1)..(2): 2′-O-methyl
  misc_feature (1)..(3): Phosphorothioate linkage
  misc_feature (98)..(99): T-LNA
gugugagagcgguaagauggguuuuagagcuagaaauagcaaguuaaaauaaggcuaguccguuaucaacuugaaaaaguggcaccgagucggugcuttu

SEQ ID NO: 126 - DNA, 100 bp, Artificial Sequence
  Synethetic
  misc_feature (1)..(1): G-LNA
  misc_feature (2)..(2): T-LNA
  misc_feature (98)..(99): T-LNA
gtgugagagcgguaagauggguuuuagagcuagaaauagcaaguuaaaauaaggcuaguccguuaucaacuugaaaaaguggcaccgagucggugcuttu

SEQ ID NO: 127 - RNA, 100 nt, Artificial Sequence; identical to SEQ ID NO: 54
  Synthetic
  misc_feature (1)..(2): 2′-O-methyl
  misc_feature (1)..(3): Phosphorothioate linkage
  misc_feature (6)..(6): G-LNA
  misc_feature (98)..(100): Phosphorothioate linkage
  misc_feature (98)..(99): 2′-O-methyl

SEQ ID NO: 128 - RNA, 100 nt, Artificial Sequence; identical to SEQ ID NO: 54
  Synethetic
  misc_feature (1)..(2): 2′-O-methyl
  misc_feature (1)..(3): Phosphorothioate linkage
  misc_feature (10)..(10): G-LNA
  misc_feature (98)..(99): 2′-O-methyl
  misc_feature (98)..(100): Phosphorothioate linkage

SEQ ID NO: 129 - RNA, 100 nt, Artificial Sequence; identical to SEQ ID NO: 54
  Synethetic
  misc_feature (1)..(2): 2′-O-methyl
  misc_feature (1)..(3): Phosphorothioate linkage
  misc_feature (12)..(12): 5mC-LNA
  misc_feature (98)..(99): 2′-O-methyl
  misc_feature (98)..(100): Phosphorothioate linkage

SEQ ID NO: 130 - RNA, 100 nt, Artificial Sequence; identical to SEQ ID NO: 76
  Synethetic
  misc_feature (1)..(3): Phosphorothioate linkage
  misc_feature (1)..(2): 2′-O-methyl
  misc_feature (6)..(6): A-LNA
  misc_feature (98)..(100): Phosphorothioate linkage
  misc_feature (98)..(99): 2′-O-methyl

SEQ ID NO: 131 - RNA, 100 nt, Artificial Sequence; identical to SEQ ID NO: 76
  Synethetic
  misc_feature (1)..(2): 2′-O-methyl
  misc_feature (1)..(3): Phosphorothioate linkage
  misc_feature (10)..(10): 5mC-LNA
  misc_feature (98)..(99): 2′-O-methyl
  misc_feature (98)..(100): Phosphorothioate linkage

SEQ ID NO: 132 - RNA, 100 nt, Artificial Sequence; identical to SEQ ID NO: 76
  Synethetic
  misc_feature (1)..(2): 2′-O-methyl
  misc_feature (1)..(3): Phosphorothioate linkage
  misc_feature (12)..(12): G-LNA
  misc_feature (98)..(99): 2′-O-methyl
  misc_feature (98)..(100): Phosphorothioate linkage

SEQ ID NO: 133 - RNA, 42 nt, Artificial Sequence
  Synethetic
  misc_feature (1)..(3): Phosphorothioate linkage
  misc_feature (1)..(2): 2′-O-methyl
guaacgcgaacuacgcggguguuuuagagcuaugcuguuuug

SEQ ID NO: 134 - RNA, 42 nt, Artificial Sequence
  Synethetic
  misc_feature (1)..(2): 2′-O-methyl
  misc_feature (1)..(3): Phosphorothioate linkage
cccggacucggacgcgugguguuuuagagcuaugcuguuuug

SEQ ID NO: 135 - RNA, 42 nt, Artificial Sequence
  Synethetic
  misc_feature (1)..(3): Phosphorothioate linkage
  misc_feature (1)..(2): 2′-O-methyl
gcggcccggagccuacgaggguuuuagagcuaugcuguuuug

SEQ ID NO: 136 - RNA, 42 nt, Artificial Sequence
  Synethetic
  misc_feature (1)..(2): 2′-O-methyl
  misc_feature (1)..(3): Phosphorothioate linkage
agggcccggacucggacgcgguuuuagagcuaugcuguuuug

SEQ ID NO: 137 - RNA, 42 nt, Artificial Sequence
  Synethetic
  misc_feature (1)..(3): Phosphorothioate linkage
  misc_feature (1)..(2): 2′-O-methyl
agggggcggauacugacccgguuuuagagcuaugcuguuuug

SEQ ID NO: 138 - RNA, 42 nt, Artificial Sequence
  Synethetic
  misc_feature (1)..(2): 2′-O-methyl
  misc_feature (1)..(3): Phosphorothioate linkage
auacugacccgaggacgccgguuuuagagcuaugcuguuuug

SEQ ID NO: 139 - RNA, 42 nt, Artificial Sequence
  Synethetic
  misc_feature (1)..(3): Phosphorothioate linkage
  misc_feature (1)..(3): 2′-O-methyl
ggugguggcugaguccguggguuuuagagcuaugcuguuuug

SEQ ID NO: 140 - RNA, 42 nt, Artificial Sequence
  Synethetic
  misc_feature (1)..(2): 2′-O-methyl
  misc_feature (1)..(3): Phosphorothioate linkage
aaccaacccccaggcgguggguuuuagagcuaugcuguuuug

SEQ ID NO: 141 - RNA, 44 nt, Artificial Sequence
  Synethetic
mcmucaaccaacccccaggcggguuuuagagcuaugcuguuuug

SEQ ID NO: 142 - RNA, 42 nt, Artificial Sequence
  Synethetic
  misc_feature (1)..(3): Phosphorothioate linkage
  misc_feature (1)..(2): 2′-O-methyl
ccucgcagagcucggagacgguuuuagagcuaugcuguuuug

SEQ ID NO: 143 - RNA, 42 nt, Artificial Sequence
  Synethetic
  misc_feature (1)..(2): 2′-O-methyl
  misc_feature (1)..(3): Phosphorothioate linkage
gcagccauagggagccgcacguuuuagagcuaugcuguuuug

SEQ ID NO: 144 - RNA, 42 nt, Artificial Sequence
  Synethetic
  misc_feature (1)..(3): Phosphorothioate linkage
  misc_feature (1)..(2): 2′-O-methyl
ggauggcggccccuaaccggguuuuagagcuaugcuguuuug

SEQ ID NO: 145 - RNA, 42 nt, Artificial Sequence
  Synethetic
  misc_feature (1)..(2): 2′-O-methyl
  misc_feature (1)..(3): phoshorothioate linkage
cagagcgucgggauaucgggguuuuagagcuaugcuguuuug

SEQ ID NO: 146 - RNA, 74 nt, Artificial Sequence
  Synethetic
  misc_feature (72)..(74): phosphorothioate linkage
  misc_feature (72)..(73): 2′-O-methyl
aacagcauagcaaguuaaaauaaggcuaguccguuaucaacuugaaaaaguggcaccgagucggugcuuuuuuu

SEQ ID NO: 147 - RNA, 104 nt, Artificial Sequence
  Synethetic
  misc_feature (102)..(104): phosphorothioate linkage
  misc_feature (102)..(103): 2′-O-methyl
aacagcauagcaaguuaaaauaaggcuaguccguuaucaacuuggccaacaugaggaucacccaugucugcagggccaaguggcaccgagucggugcuuuuuuu

SEQ ID NO: 148 - RNA, 99 nt, Artificial Sequence
  Synethetic
  misc_feature (97)..(99): phosphorothioate linkage
  misc_feature (97)..(98): 2′-O-methyl
aacagcauagcaaguuaaaauaaggcuaguccguuaucaacuugaaaaaguggcaccgagucggugcgcgcacaugaggaucacccaugugcuuuuuuu

SEQ ID NO: 149 - RNA, 98 nt, Artificial Sequence
  Synethetic
  misc_feature (77)..(77): 2ADP
  misc_feature (96)..(97): 2′-O-methyl
  misc_feature (96)..(98): phosphorotioate linkage
aacagcauagcaaguuaaaauaaggcuaguccguuaucaacuugaaaaaguggcaccgagucggugcgcggcccggggaucaccacgggccuuuuuuu

SEQ ID NO: 150 - RNA, 42 nt, Artificial Sequence
  Synethetic
uuaauuucuacucuuguagauagagugccuagaaagaugaca

SEQ ID NO: 151 - RNA, 42 nt, Artificial Sequence
  Synethetic
uuaauuucuacucuuguagaucuggacgggggacaggcugug

SEQ ID NO: 152 - RNA, 42 nt, Artificial Sequence
  Synethetic
uuaauuucuacucuuguagauucagauaacugggccccugcg

SEQ ID NO: 153 - RNA, 42 nt, Artificial Sequence
  Synethetic
uuaauuucuacucuuguagaucccccuccggcucggcgccgg

SEQ ID NO: 154 - RNA, 42 nt, Artificial Sequence
  Synethetic
uuaauuucuacucuuguagaugccuccgccuguggaugcugc

SEQ ID NO: 155 - RNA, 54 nt, Artificial Sequence
  Synethetic
  misc_feature (1)..(2): 2′-O-methyl
  misc_feature (1)..(3): phosphorothioate
  misc_feature (52)..(54): phosphorothioate
  misc_feature (53)..(54): 2′-O-methyl
cuuucaagacuaauagauugcuccuuacgaggagacagagugccuagaaagaug

SEQ ID NO: 156 - RNA, 54 nt, Artificial Sequence
  Synethetic
  misc_feature (1)..(2): 2′-O-methyl
  misc_feature (1)..(3): phosphorothioate
  misc_feature (52)..(53): 2′-O-methyl
  misc_feature (52)..(54): phosphorothioate
cuuucaagacuaauagauugcuccuuacgaggagaccuggacgggggacaggcu

SEQ ID NO: 157 - RNA, 43 nt, Artificial Sequence
  Synethetic
  misc_feature (1)..(2): 2′-O-methyl
  misc_feature (1)..(3): phosphorothioate
  misc_feature (41)..(43): phosphorothioate
  misc_feature (42)..(43): 2′-O-methyl
aauagauugcuccuuacgaggagaccuggacgggggacaggcu

SEQ ID NO: 158 - RNA, 39 nt, Artificial Sequence
  Synethetic
  misc_feature (1)..(2): 2′-O-methyl
  misc_feature (1)..(3): Phospohorothioate
  misc_feature (37)..(39): Phospohorothioate
  misc_feature (37)..(38): 2′-O-methyl
aauagauugcuccuuacgaggagaccuggacgggggaca

SEQ ID NO: 159 - DNA, 113 bp, Artificial Sequence
  Synethetic
gtaacgcgaactacgcgggtgtttaagagctatgctggaaacagcatagcaagtttaaataaggctagtccgttatcaacttgaaaaagtggcaccgagtcggtgcttttttt

SEQ ID NO: 160 - DNA, 113 bp, Artificial Sequence
  Synethetic
ggattgttgcgtcccatatcgtttaagagctatgctggaaacagcatagcaagtttaaataaggctagtccgttatcaacttgaaaaagtggcaccgagtcggtgcttttttt

SEQ ID NO: 161 - DNA, 113 bp, Artificial Sequence
  Synethetic
gctcagtcgggcaagagtcggtttaagagctatgctggaaacagcatagcaagtttaaataaggctagtccgttatcaacttgaaaaagtggcaccgagtcggtgcttttttt

SEQ ID NO: 162 - DNA, 113 bp, Artificial Sequence
  Synethetic
gactgggcctgaaagggtacgtttaagagctatgctggaaacagcatagcaagtttaaataaggctagtccgttatcaacttgaaaaagtggcaccgagtcggtgcttttttt

SEQ ID NO: 163 - DNA, 113 bp, Artificial Sequence
  Synethetic
gcgccgccggcccagctatagtttaagagctatgctggaaacagcatagcaagtttaaataaggctagtccgttatcaacttgaaaaagtggcaccgagtcggtgcttttttt

SEQ ID NO: 164 - DNA, 113 bp, Artificial Sequence
  Synethetic
gcggcagcagtagcggtcacgtttaagagctatgctggaaacagcatagcaagtttaaataaggctagtccgttatcaacttgaaaaagtggcaccgagtcggtgcttttttt

SEQ ID NO: 165 - DNA, 113 bp, Artificial Sequence
  Synethetic
ggggggcggatactgacccggtttaagagctatgctggaaacagcatagcaagtttaaataaggctagtccgttatcaacttgaaaaagtggcaccgagtcggtgcttttttt

SEQ ID NO: 166 - DNA, 112 bp, Artificial Sequence
  Synethetic
ggtggtggctgagtccgtgggtttaagagctatgctggaaacagcatagcaagtttaaataaggctagtccgttatcaacttgaaaaagtggcaccgagtcggtgctttttt

SEQ ID NO: 167 - DNA, 113 bp, Artificial Sequence
  Synethetic
ggtggtggctgagtccgtgggtttaagagctatgctggaaacagcatagcaagtttaaataaggctagtccgttatcaacttgaaaaagtggcaccgagtcggtgcttttttt

SEQ ID NO: 168 - DNA, 113 bp, Artificial Sequence
  Synethetic
gcacagcctgtcccccgtccgtttaagagctatgctggaaacagcatagcaagtttaaataaggctagtccgttatcaacttgaaaaagtggcaccgagtcggtgcttttttt

SEQ ID NO: 169 - DNA, 113 bp, Artificial Sequence
  Synthetic
gatccgctaggcgcaccgacgtttaagagctatgctggaaacagcatagcaagtttaaataaggctagtccgttatcaacttgaaaaagtggcaccgagtcggtgcttttttt

SEQ ID NO: 170 - DNA, 2046 bp, Artificial Sequence
  Synthetic
atggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctacagcgctggaggaggtggaagcggaggaggaggaagcggaggaggaggtagcggacctaagaaaaagaggaaggtggcggccgctggatccatgagtaggagaaaacaagcaaaaccacagcactttcaaagtgatcctgaggtagcaagccttccacggcgggacggtgacacggagaagggtcaaccaagtcgacccacgaaaagcaaagatgctcatgtatgtggacgctgttgcgcagaattttttgaattgtccgatcttcttcttcacaaaaagaactgcacgaagaatcagttggttttgatagtaaacgaaaatccagcttcacccccagaaactttttccccgtcacctcctccagataatcctgatgaacaaatgaatgacaccgtaaataaaaccgaccaagtagactgttctgatttgagcgaacacaacggtttggatcgagaagagtcaatggaagtagaggccccagttgccaataagtcaggcagcggtacttcttccggctcccacagttcaacagctccatcctcaagtagttcaagctcttctagttcaggaggcggggggagtagctctaccggcacttctgccatcacaacctcacttcctcagcttggagacttgacaggatccggtgggggatctgggggatctggctcgatgtctgcagctggccttttggctcctgcccccgcacaagcgggagctcctcccgcaccggagtactatccagaagaggatgaggaactggaatctgccgaagacgacgagcgcagttgccgggggagggaatctgacgaggatactgaggatgcttctgagaccgacctcgcgaaacatgatgaggaagactacgttgaaatgaaagagcagatgtaccaagacaaacttgctagcctcaagagacagttgcagcaactgcaagaaggcacgctccaggagtaccagaagagaatgaaaaaactcgaccagcagtacaaggaacgaattagaaacgcagagctctttcttcagctggagactgaacaggttgagcgcaattatattaaggaaaaaaaagccgctgtgaaggagttcgaagacaagaaagtggaacttaaagaaaacctcatcgccgaactggaggagaagaagaagatgatagagaacgaaaaactcacaatggaactgacgggtgattccatggaggtaaaaccgattatgacccgaaagctccgccgacgcccaaacgatccggtaccgatccctgataagcggcgcaagcccgcaccggctcagctcaattacctgctgaccgacgaacaaataatggaggacctgcggactcttaataagctgaagagtcctaaacggccagcttcccccagttcccccgaacacctgcccgctactcccgcggagagccctgctcagcgctttgaggcccgaatcgaggacggaaaattgtactatgacaaacgctggtatcataagagccaggctatatacctggagtcaaaagataaccaaaagttgtcatgtgtaatctcctcagtcggggctaacgaaatatgggtgcggaagacctctgatagtacgaagatgcgcatatatctgggacaattgcaaagaggactttttgttataagacggagaagcgctgct

SEQ ID NO: 171 - protein, 1942 aa, Artificial Sequence; identical to SEQ ID NO: 42
  Synthetic

SEQ ID NO: 172 - DNA, 5826 bp, Artificial Sequence
  Synthetic
atgtctgcagctggccttttggctcctgcccccgcacaagcgggagctcctcccgcaccggagtactatccagaagaggatgaggaactggaatctgccgaagacgacgagcgcagttgccgggggagggaatctgacgaggatactgaggatgcttctgagaccgacctcgcgaaacatgatgaggaagactacgttgaaatgaaagagcagatgtaccaagacaaacttgctagcctcaagagacagttgcagcaactgcaagaaggcacgctccaggagtaccagaagagaatgaaaaaactcgaccagcagtacaaggaacgaattagaaacgcagagctctttcttcagctggagactgaacaggttgagcgcaattatattaaggaaaaaaaagccgctgtgaaggagttcgaagacaagaaagtggaacttaaagaaaacctcatcgccgaactggaggagaagaagaagatgatagagaacgaaaaactcacaatggaactgacgggtgattccatggaggtaaaaccgattatgacccgaaagctccgccgacgcccaaacgatccggtaccgatccctgataagcggcgcaagcccgcaccggctcagctcaattacctgctgaccgacgaacaaataatggaggacctgcggactcttaataagctgaagagtcctaaacggccagcttcccccagttcccccgaacacctgcccgctactcccgcggagagccctgctcagcgctttgaggcccgaatcgaggacggaaaattgtactatgacaaacgctggtatcataagagccaggctatatacctggagtcaaaagataaccaaaagttgtcatgtgtaatctcctcagtcggggctaacgaaatatgggtgcggaagacctctgatagtacgaagatgcgcatatatctgggacaattgcaaagaggactttttgttataagacggagaagcgctgctggatccggtgggggatctgggggatctggctcgatgagtaggagaaaacaagcaaaaccacagcactttcaaagtgatcctgaggtagcaagccttccacggcgggacggtgacacggagaagggtcaaccaagtcgacccacgaaaagcaaagatgctcatgtatgtggacgctgttgcgcagaattttttgaattgtccgatcttcttcttcacaaaaagaactgcacgaagaatcagttggttttgatagtaaacgaaaatccagcttcacccccagaaactttttccccgtcacctcctccagataatcctgatgaacaaatgaatgacaccgtaaataaaaccgaccaagtagactgttctgatttgagcgaacacaacggtttggatcgagaagagtcaatggaagtagaggccccagttgccaataagtcaggcagcggtacttcttccggctcccacagttcaacagctccatcctcaagtagttcaagctcttctagttcaggaggcggggggagtagctctaccggcacttctgccatcacaacctcacttcctcagcttggagacttgacaggatccggtgggggatctgggggatctggctcgatggattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaagcgtcctgctgctactaagaaagctggtcaagctaagaaaaagaaa

SEQ ID NO: 173 - protein, 132 aa, Artificial Sequence
  Synthetic
MASPADSCIQFTRHASDVLLNLNRLRSRDILTDVVIVVSREQFRAHKTVLMACSGLFYSIFTDQLKCNLSVINLDPEINPEGFCILLDFMYTSRLNLREGNIMAVMATAMYLQMEHVVDTCRKFIKASEAEM

SEQ ID NO: 174 - protein, 306 aa, Artificial Sequence
  Synthetic
MELKDYYAIMGVKPTDDLKTIKTAYRRLARKYHPDVSKEPDAEARFKEVAEAWEVLSDEQRRAEYDQMWQHRNDPQFNRQFHHGDGQSFNAEDFDDIFSSIFGQHARQSRQRPATRGHDIEIEVAVFLEETLTEHKRTISYNLPVYNAFGMIEQEIPKTLNVKIPAGVGNGQRIRLKGQGTPGENGGPNGDLWLVIHIAPHPLFDIVGQDLEIVVPVSPWEAALGAKVTVPTLKESILLTIPPGSQAGQRLRVKGKGLVSKKQTGDLYAVLKIVMPPKPDENTAALWQQLADAQSSFDPRKDWGKA

SEQ ID NO: 175 - protein, 137 aa, Artificial Sequence
  Synthetic
MSEALKILNNIRTLRAQARECTLETLEEMLEKLEVVVNERREEESAAAAEVEERTRKLQQYREMLIADGIDPNELLNSLAAVKSGTKAKRAQRPAKYSYVDENGETKTWTGQGRTPAVIKKAMDEQGKSLDDFLIKQ

SEQ ID NO: 176 - protein, 291 aa, Artificial Sequence
  Synthetic
MERKRWECPALPQGWEREEVPRRSGLSAGHRDVFYYSPSGKKFRSKPQLARYLGGSMDLSTFDFRTGKMLMSKMNKSRQRVRYDSSNQVKGKPDLNTALPVRQTASIFKQPVTKITNHPSNKVKSDPQKAVDQPRQLFWEKKLSGLNAFDIAEELVKTMDLPKGLQGVGPGCTDETLLSAIASALHTSTMPITGQLSAAVEKNPGVWLNTTQPLCKAFMVTDEDIRKQEELVQQVRKRLEEALMADMLAHVEELARDGEAPLDKACAEDDDEEDEEEEEEEPDPDPEMEHV

SEQ ID NO: 177 - protein, 255 aa, Artificial Sequence
  Synthetic
MDAKSLTAWSRTLVTFKDVFVDFTREEWKLLDTAQQIVYRNVMLENYKNLVSLGYQLTKPDVILRLEKGEEPWLVSGGGSGGSGSSPKKKRKVEASVQVKRVLEKSPGKLLVKMPFQASPGGKGEGGGATTSAQVMVIKRPGRKRKAEADPQAIPKKRGRKPGSVVAAAAAEAKKKAVKESSIRSVQETVLPIKKRKTRETVSIEVKEVVKPLLVSTLGEKSGKGLKTCKSPGRKSKESSPKGRSSSASSPPKKE

SEQ ID NO: 178 - DNA, 3906 bp, Artificial Sequence
  Synthetic
atggtcgacggtaagcctatccctaaccctctcctcggtctcgattctacgccaaaaaagaagagaaaggtcaataacggaactaataacttccaaaacttcatcgggatcagttccttgcagaaaactctccggaatgctctcatcccaactgagactactcagcagttcattgttaagaatggaatcataaaagaggacgagcttaggggggaaaataggcaaatcctcaaggatatcatggatgactattataggggctttatatccgagacactgagcagcattgatgatatagactggacctctcttttcgaaaagatggaaatacaacttaaaaatggagataacaaggacaccctgataaaggaacagaccgaatataggaaggcaattcataaaaagtttgctaacgatgataggtttaaaaacatgttctcagcaaaactcatttcagatatactgcccgaattcgttatccacaacaacaactactccgctagcgaaaaagaggaaaagacccaagtcataaagctgttctctcgattcgcgacgagttttaaagattatttcaagaatcgcgcaaactgtttctcagctgatgatatcagcagctcatcctgtcatcggatcgttaacgataatgctgaaatcttcttctccaatgcacttgtttataggcgcattgttaaatctctctcaaacgatgatatcaataagatttccggcgatatgaaggacagtcttaaggagatgagcctcgaagagatatactcatacgagaaatatggcgaatttatcacccaggaagggatttccttctataatgacatttgcggcaaagtcaattccttcatgaacctgtattgccaaaaaaataaagaaaacaagaacctctataagctgcaaaagttgcataagcaaatactttgtatcgcggatacaagctatgaagttccctacaagttcgagagtgatgaggaggtgtatcaatctgtcaatggtttccttgataatatttcttctaagcatattgttgaacgactccgaaagataggagacaactataatggatacaatttggataaaatctacatcgtgtctaaattttacgagagtgtgtcacaaaaaacatatagagactgggagacaattaataccgccctggagatacattacaacaatatacttcccgggaacgggaagtctaaggcagacaaggtgaagaaagccgtgaagaacgacttgcaaaagtcaattaccgaaatcaatgagcttgtttcaaactataaactttgttcagatgacaatattaaagccgaaacctatattcatgaaatctctcatattctgaataactttgaggcgcaagaactgaaatataacccagaaatacacctcgttgagtccgaactgaaagcaagcgaactgaaaaatgttttggacgtgataatgaacgcttttcattggtgctcagtctttatgacagaggagcttgttgacaaggataacaatttctatgcggaactggaagagatttacgacgaaatctatccggtcatatccctgtataacctggttcgcaactatgtcacgcaaaaaccatacagcacgaagaagattaaactgaactttggtattccgacgctggccgatggatggtcaaaatctaaggaatactcaaacaatgccataatcctgatgcgagataacctctactaccttggaatctttaatgctaaaaataaacccgataaaaaaattatcgaagggaacacgagtgaaaacaaaggtgattataaaaaaatgatatataatctgcttccaggaccaaataagatgatacccaaagttttcctttcttcaaagaccggcgtcgagacatataaaccatccgcgtacatacttgaaggctacaaacaaaataaacatatcaaatcatctaaggattttgacattacgttctgtcatgatttgattgactatttcaaaaattgcatagccattcatccagagtggaaaaactttgggtttgacttctctgataccagtacatatgaagacataagtggattttaccgagaagtagagctccaaggttataaaatagactggacctatatatctgaaaaggatatagaccttttgcaagagaagggacagctttatcttttccaaatctacaacaaagacttcagtaagaaaagtaccgggaatgacaatcttcataccatgtatctgaagaacctgttctccgaagaaaatctgaaggacatagtcctgaagcttaatggcgaagcggaaatttttttccgaaagagctctattaagaaccccataatacataagaagggaagcattctcgttaatcgaacgtatgaggccgaagagaaagatcaatttgggaatatccaaatcgttcgaaagaacataccagaaaatatttaccaagaattgtacaaatattttaacgataaaagcgacaaagaactgtctgatgaagctgctaagctgaaaaacgtcgtcggccatcatgaggccgcgacgaatatagtcaaggattaccgatatacatacgataagtatttcctgcatatgcccatcactatcaactttaaggcaaataagactggattcattaatgacagaatactgcaatacatagctaaagaaaaagatttgcatgttattggcattgccaggggtgagcgcaatcttatctatgtaagcgtcattgatacttgcgggaatatcgtagagcagaagtcatttaatattgtaaatgggtacgattaccaaatcaagttgaagcagcaagagggagcacgacagattgcccgcaaggagtggaaagagatcggaaagataaaggagatcaaggaggggtatttgtcccttgttatacacgaaatttccaagatggtaatcaagtacaacgctataattgctatggcggatctctcctatggatttaaaaagggaagatttaaagtcgagcggcaggtatatcagaaatttgaaacaatgcttattaataaacttaattatctcgttttcaaagacattagtatcaccgaaaacggtgggctgttgaagggctatcaacttacgtacataccagataagcttaagaatgtgggtcaccaatgcggatgcatattctacgtgcccgcagcttatacaagcaaaatcgacccaacaacgggtttcgtaaacatatttaagttcaaggatctcaccgtggatgccaagcgagagttcataaaaaaatttgactcaatcagatatgactcagaaaagaatcttttttgttttaccttcgactacaataatttcattacacaaaatacggttatgagcaagtcatcctggtccgtatatacgtatggagtgcgcataaagcggagattcgttaacgggcgattttctaatgagtccgatacaatcgatataacaaaggatatggaaaaaactctggaaatgactgatataaattggagggacggtcatgacctcaggcaagacattatcgattatgagatcgtgcaacatatttttgagatctttcggttgactgtccaaatgaggaactctctgtctgaattggaagatagggactacgatcgcctgataagccccgtgttgaacgagaataacatattctacgattccgcgaaagccggggatgcgctccctaaggacgccgctgcaaatggggcctattgtattgctttgaaagggctgtacgaaatcaaacagatcaccgaaaactggaaagaagacgggaagtttagtcgggataaactgaagatatccaacaaggactggtttgactttatccaaaataagcgatatttgaagcgtcctgctgctactaagaaagctggtcaagctaagaaaaagaaa

SEQ ID NO: 179 - DNA, 2250 bp, Artificial Sequence
  Synthetic
atggtcgacgggagcgggccggcagctaaacgggtgaagttggacagtggtggaattaaacctacagtttctcagtttcttacccctggttttaagctgataagaaaccatagtcggacggctggacttaagctgaagaatgagggcgaagaggcatgcaagaagttcgtacgggagaacgaaattcccaaagatgaatgtccaaactttcaaggtggacccgcaatcgcgaacattatagccaagagtcgcgaatttaccgagtgggaaatatatcaaagttcactggcgatccaagaggtgattttcaccttgccgaaggataagctgcccgagcctatactcaaggaagaatggcgcgcccaatggttgagcgaacacggcctcgatacggtgccttacaaggaagctgccggacttaatttgataattaagaacgcggtcaacacttacaaaggggtccaggtgaaagtcgataataagaataagaacaacctggccaaaatcaaccgcaagaacgaaatcgcgaaattgaacggcgaacaagaaatcagcttcgaagagatcaaagccttcgatgataaaggatatctcctgcaaaagccaagtccgaataagagcatatattgctaccaaagcgtgtctccaaagccattcataacctctaaataccataacgtgaatctgcccgaagaatatatcggctactaccgcaagtcaaacgagcccatcgttagtccctatcaattcgatagattgcgaatcccaattggcgaacccggatatgtaccaaaatggcagtatacctttctgtctaagaaagagaataagcggagaaagctctccaagcggattaagaatgttagtcctattcttgggataatatgcattaagaaagactggtgcgtattcgatatgaggggcctgctcagaacgaaccactggaagaaataccataaaccgacagattctatcaatgacctcttcgattatttcactggagaccctgtaatcgacacgaaagcgaacgtcgtccgattcagatataaaatggaaaatggcattgttaattacaagccggtgcgcgaaaagaaaggcaaggaacttttggaaaacatatgtgatcaaaatgggagctgtaagttggccactgtggccgttggtcaaaacaacccagtggcaattggactgtttgaacttaagaaagtaaatggtgaacttaccaaaaccttgatttcacggcatcctactccgatcgacttttgtaataaaattacggcttacagggagcggtatgataagctcgaatccagcatcaagttggatgccataaagcaattgacatctgagcaaaagatcgaagttgataactataacaataattttacccctcaaaacactaagcagatagtgtgcagcaagctcaatatcaatccaaacgaccttccttgggataaaatgatttctgggactcatttcattagcgagaaagcccaagtcagtaataaatcagaaatatacttcacatctaccgataaggggaaaactaaggacgtaatgaagagcgactacaagtggtttcaagactataaaccaaaactgtcaaaggaagtaagggacgcactcagcgatattgaatggcggcttaggagagaaagtcttgaatttaacaaattgagtaaatcacgggaacaagatgcacggcaactggccaattggatctcttccatgtgtgatgttatcggaatagagaacctggtgaagaagaacaatttctttggtggaagcggcaagagggaaccggggtgggacaacttctataaaccgaagaaggagaatcgatggtggatcaacgcaattcataaagctctcacagaactctctcaaaacaaagggaaaagagtgattctcttgccagcaatgagaacatctatcacatgccctaaatgtaagtactgtgacagcaagaaccggaacggcgagaagttcaattgtctgaagtgtggcatagaactcaacgcagacattgatgttgctaccgaaaatctcgcgaccgttgctattaccgcgcaaagtatgcctaaacccacctgtgagaggagtggtgatgccaagaagcccgtacgtgcacgaaaggcaaaggcgccagaatttcatgacaaactcgcgccctcatacacagttgtcttgcgcgaagctgttaagcgtcctgctgctactaagaaagctggtcaagctaagaaaaagaaa

SEQ ID NO: 180 - DNA, 19 bp, Artificial Sequence
  Synetheic
atgctgtctaagacagcat

SEQ ID NO: 181 - protein, 133 aa, Artificial Sequence
  Synthetic
MAKLETVTLGNIGKDGKQTLVLNPRGVNPTNGVASLSQAGAVPALEKRVTVSVSQPSRNRKNYKVHVKIQNPTACTANGSCDPSVTRQAYADVTFSFTQYSTDEERAFVRTELAALLASPLLIDAIDQLNPAY

SEQ ID NO: 182 - protein, 1409 aa, Artificial Sequence
  Synthetic
MDYKDDDDKMAPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKK

SEQ ID NO: 183 - DNA, 4227 bp, Artificial Sequence
  Synthetic
atggattacaaagacgatgacgataagatggccccaaagaagaagcggaaggtcggtatccacggagtcccagcagccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaagcgtcctgctgctactaagaaagctggtcaagctaagaaaaagaaa

SEQ ID NO: 184 - DNA, 1566 bp, Artificial Sequence; identical to SEQ ID NO: 4
  Synthetic